SEQUENCE LISTING (50 sequences)

SEQ ID NO: 1 - DNA, 2766 bp, Trichoderma reesei
atggctgcagagggtaatacgaatgatggacggagtcggcaggaatctgttggcacctcgtccactggacaacagcttccatcgttgagtagcctctttggcccgccatcagctatgagacctctgagctctccacacacggagcgcaatggaatatatccttcgccctctcccctagacaggccacggctatcgtcgaatggaaacctgtcgaactcctacttcccacagactatttcaccttctccgctgcaaccgagaagtctctatgacacagtctacaactaccacgatagacagtccgcccacgccccgacatcttccttcactgggccgcagtcgcccgaatactcgaaccacgatcacaggggctcagatgcgcgttacgaacccgaaatgccgcgcaaatggtccgcctaccgcgaggacagcaggcaagattatcaggcggcgggatctcgcgactcatcttatccgcaaggtcaagatcaactcaggactcatctctctgggcacaaagatgcaacccacgattactccgaacggcgacccagtcaagttcccagttcggacggcaacccgccagcaactacagctaccaccataccctctgatatcgccgtttccaaagatggcctgggcccaaaaatctggacaggcacgcacttcctaccgagatttgtgcgagcagcagaggtaccgggcgaaggcatgtgctatttctacgacgatggaagccattgcaagaccgtcattgacggtgaggcggtaaacgctcactggggcgtgacgaaggcgggcaagcctcgcaagcggttggctattgcatgcgtgacttgcagggagaagaagatcaagtgtgacccggactacccacgatgtgtccagtgcgaaaagtttggtcgcatttgcaaattcaagaatgcgtgagtgcatgaaccatgcttttctacatcaagatgccctcggaatcgtgctcacagtgattctaggcccagagggggcaataacacttcgccctcgacgtcgccagtcgagctcgacgatggccgacaactcgaagcagctgatctacggcattcgatcagtgatgtgagctctcccagatcgccgcgagcaatgcaaccctcatctccagatcgcggatacagcaagaggctcaagctgggatctgaggcgtacgtttccaacggcgaaccgcctgcctcgttcaatcgccgattggactatctcaaacctcgaatggccgaacagccaccgctaccacttctccccgagccatctaggatcccggacgatgttcttggtcgtgcttggcggaccgatccctttgcctcgaaccctgacctgatcaccagtgccctgaccaggtttttcgccaatgtggacagcactatgattctacaatttctcccggaggaggcctttcagcgctgggtcgtggcttctgttggacaaaaatcccctgaagacttgatgctcctctatagcacccttgccgttggctctgcgctctgcggcgatcctaagcatattgctttcgaatatgctcaggtggctcactacgcgcaaaggatgacgggagtgcagtgcttacagctggtgcaaagcaggatcctccttgccgtgtattacatctcgacctgccgtacacgagaagccgatgagctgatatcttcagcagcggctagcgctgcatgcctgcagctgagcttagaactcgaccactcccgggaatcggccatgaccatttatcctctgggactcaataggatgggctatgctgaagctcgcaggaggactctttggtcactgttcatgctggaaagacttggcgggcttttcccggaacgcccagtaatgactcatgcagaggacatttacattcgacttcctgccgacctcaatagcttcgagaagcaagccgagtcccgcgcgcgaagctttgacccatattgtggccgcgtcgtgtcggatgagccatcgtcggacgccactgtcacgggctactatcttgaaatggtgcacatctggtcggactgccaggcctccatctatagaatggccctcagacggacgcccaccgaagccgagaccacgaagctgcaggatctcatcaagagagccaagaactgggctgcttctctaccccctcgcatgacgtttagtggcacaaacatcgaaacggctgctttctcgagaagcacggggtctttcctcagcatgcattttctttaccatcataccatgatcgagttgaaccgacaccgctacggagccggccagctaccccgggatgtccagttggggcattcggccgagtgccgggagcacgcaagccgggttctcgagatgctgaacagtcttgagcgtatcttgagggtcaggtctgcattgctgagcatccctcctccagccatggctgtcgccgtcacagaagctgtcgatgttctgactgccagtggtccgatggccgttttaggcgagatcattgaccgagttcgaatcgcccagtcggcaattgacaagatgaaggacgtctgggaggcatcctcaaaggacggtcttgccatccaccggcgtttacaaaagttgaaccgcattcgtgagctaggatcgcggccgcccagccctattcagggctatcgcttgcttcccctgtcagacaacacaaaggacaaggagctcagccactggcagatctttgatccccttgaacaaacatttcccagagacatggacgtggtctatgtcggatgcgactag

SEQ ID NO: 2 - DNA, 3716 bp, Trichoderma reesei
atgcgattagacaagttcaacaaggcacccttcacgcccatcaagtcgtctcccgtggccacagtacagccgcgaagcgtacccaacgattctcccctcaagctgagcccgcagacgactgctgaaaggcccgacgtcaagagcgaggccaacggcaagccggtcagccaaacgcagccgattccagtcaatggctcgagcggcgcccggcgtctgagggcgggctccgtcgacaagcagaccagcagcagcgtgacgccgcctcaccgtcgcaactcgtggttctccaacatctcggccaagttctcaggctcagtcccccaggcgaacggccagacgcagcagacgggcaatcagcagcatgcgccttcacaacagcaccagccggcggccccaacaaacgcagaacccgttgtgcccaaaattacgccgaccaagaacgccgtcctccagcatgggttaaaaccggagggcgacgagccatacacgccggccccgccgagatcgggccagcctggactgctcggcgtctttcggcgcctttcttcctcaggcggtggaagcagtggtgctcttggcgctgccggcaaattcaatcacggtctggttgagcgccggatattgaacgtggaccgggatcgagaaagatgcccgattgcagagctcaaggacaacaagctgaggcgggtgtccttcttcgtcgacgtcgaggttgcgcccatgccaaagtacgcagaaacagaggcagacaccgattccatcgaccacacccagaagaagaaactgagcgagaagggcgagggcgaggctttgaaacaccccaaggttgccgaagcccaaaaggaggcttgctcatcttcgactgagagtgtcagccccggcgccgctgaagcgaaaccacgcgaaaccgagaccaagcaagccgcaccgacacagaatggcacaaagacggcggacagcccggaagaaaccaaagccgctccaaaggacatgacgagaaagaaggagaagaagaagagaagtgaagaggagcgcaaggccaggaaagagaagaggcggcggctcgccgaagagagcggagcgattcccatagagattcactatgacagcagcgactctgccgagctgcacggtcccccctccaagacgaactcgcccacaatcaacccggtcagaatctaccgaagatgctgccagcttcgcgagacccccattctcaagaagattaccgagcagctgagcgacacggccaacattctccccaatgggtgcgtgaacaagattgacttgaccggctacttcatgcagctgcaagatctcatcaccctgggcgactatcttgccgtcgtgtccgtgaaggaagtcattctgcaaaacagcgggctcactgacgaaggcgtcagagtcattctagctggtctcctggcagccaagcacacagagccggggaggcggaggaggaattccaatgcccatcatgagcatacgagcggagtcgtggagagggtcgtgctgaaggaaaacaagctggggccagacggctggaagcacatttcgctcttcatctacaagtgccggtcgctcaagtacctggatctctcgagcatccccttcccccgccaagctcccgccaaccccaacggcacgcttcagaacggcgtccacatcccgctcaccattgccgacatcttttccaaggcactctcagatcgcccagccgggcccgcgctcgaaatgctcagcgtcggcgagacggagcccagcatggagcagctgggcaagatcatcgacggagtgatcaagtgcggcgtgaagcgactcagcactgcccggaatcccctcgacagcgacggcgtccagcacgttgtgaggtatctggagtctggaaagtgcgaaggcctggacctgggcggcatcgatctcagggagcacatggacaccattgcggcgtccatcaaggagacggatccgctttgggcgctgagcatctccgactgcaacctgacgccctcgtctctgcgcaacattctgcccgttttggccaagctgaagaatttcgtcttcatcgacctttcccacaatcatgatctcttccaatccacgccgagtgctgttggtcttctcagaaggtatgttggggggggatgtgtagtgattatacaggaagagaggaaactgacacttcacacagatatattcccaaaatgcaatgcctcaagcggctgcatctggcggatgtcaacatgacggctgagcaagcgattgccattgtcgaagtcgtgccggaagcccgccatctctgccacctgagtctgctggggaacacggagattgtggcccttgcgagcgccaagacggaggaagcgcaagaagaagcctgcgccttgtatgcgtcgctgatggctgccactaggctgtccaagagcttgatttgcgtggacattgaggtccccagcgaggaggctggcgagatcgtcaaggcaatggcaaagcaggttgtggcctactgcctgcagaacattgagcacctccacgatgtcgagatcaacgaggcggtggccacagcgctagccgaggccaagggagagtctctggatagcaagaccccgacgtatcccgatgtgctggcgcatctcgttggccacgacgtcctagaggaggacgagctggaggaggacggctccgggccggacgaggactacgtcattggtggtaccggcgttgtcaaggcgcttgcgtgctgcctcaagaaccgcggcgacgaatctcgccgggcttctggcgagtttttccgggcaggcggcgtcgaggaagagcatcttgtcggccccaagatcccgacgacgagcggcaaggccaaggacctgtcgaagcacttgctggccggtgctcgcaagatccgacagcgtctccagccggcactgagcaaggcgagagcgaatccgagcgacgagctgaacctgcgcaagctcaacttcctggacgagacgctgcagggcatcatcaagcgcttcgaggacgagtatcccgacactcgcgagcacgcggaagaaagccaggtgtcatcccagccggaggcggaggtgctctcgtcatctccaccccccctggacgacgccgcggcaggcggatcagaccccgaagacgagggcgagctcagcgtgcggcccacgctgtcacggaacaactcgatcctgggcaagcagctcgccgaggaagaaggacgggtacaccgtgccggtcaccgattccgatctgcctttacggagcacatggacctcctcacgaccattgacgacgtggaaaaggacccaaagcacgcgcatgtgcttaacgagattgccgaagaggttggcggggagttcctggaactggccaagaagaagggtgccgtccaggctttcaaggagaatagggatattctctttgcagccttgaaagagagcgatcccgagtattgggagcgcttcgtcgaagcgcagcgcaaggcacgagccaacatcaacttgccggctgcggagaagaacgaggaaagactgcgacaggcccaggcagacgagagcgccattgccgattagttgacgggcagcgcatgatttgtgtttctgttcgcgactacccccttattctggtgctttacttgatccccaaattttgtttataccatcgttttttgtttctttttcccccatggaggatttttgggattttggata

SEQ ID NO: 3 - DNA, 13364 bp, Trichoderma reesei
atggaagtgacctcggccaccgccccgagcctcggcgccgccagcagcaacggcgcctcgctgcccgcgccgccgtcgcccacaccgacgccgacgcccttcccgaccatcgaccccgaacgcgtcgtcgagcacctcgtcgccatctgcgaggtcgccctcggcgcatcgcgcagcgacctcgagctgccgggcaacctgctgcacaaggcctcgtacgcagagaccgtctcgcggtgcacgcgcttcgccaacgactccctcaatgtgctctacatacaaaaggacctggtgcaggccggcgcgctggagaatggcaacgacgccgccgatgcgggcgagcatagcaatggcgttgctggtgaggatttctgtaacttctctctctctctccctccccactctctccctccccatctatccatctccctctcccacccccccgaaactccatctgttcccccgaatgttaaagaagggcctggtgacatctggctgacattgctctctctctgtctttggttgatatagctcccgctacctacgactacaccctctcgaccgaaatctcctcttcacccacgactgtcgccactctgattttgctcaaggcatcgcaagcgattgattcttctcgtcctcttacctcgcagatattcatcaccaacctacccggtcccgcctccctcaatgccgccgccggagagcagggcgttgcgctctccccctgggaggtgctccattcccaggtccaccacgccctggtgccctacttcgacgccaacagcaagagtcagctgctggccaatgggtccaggggaagggacgtcgatgccaagacgggcattcccgtcaccaagaagcgcctcaacgacctggagctgagcctgctccacctgcagcagaacgtcgacattcccgagatctcgctgacgttccactcgctcattcagaacgtcctcgacgatgccgaggtcaccggtacgaagccctccattgaggccatccccaagaacctcctccaggacagcagcttcctcaacaagctgcaggccaacgtcaacacctggatcaagtccatccagggcatcaccaagctgaccaaggagacctcgtccaacgccgtccaggagttcagcaccgccagccaagaggtcaacttttggctctccatggagtctgcgctcgagggcatcgaggaccagcttaggagcgagggcgtcctcctcaccctcgatattctcaagcacgccaagcgtttccaggccaccgtcagcttcgtcgccgacacgggcctcaaggaggccaaggaaaaggcgcaaaagtataaccagctcatgcgcgatttccccctcgacgagctgctgtccgctccgtcgctgctcaaggtcgaggaagccattactcagatctttgcccacctcatgaagaagctgagggtgtgtccctaccctatccggcgagcactgagcttggcccaggccatctcggcggatctcaacgacgtgctgctccgcttgcttccgggcactgagctggtcaacatgggctacccagagttccagaacgtcatgcggacctgcgacagcatctttgccgcctgggaagacaacatcaaggagtttacgcacctggctcgtatgctcatcatccgcagaaacgagaagcgcatccccatcaacgtcgagaagaaccactctgagctggagtcgcgcatcaagtacgtcagcgcattccgagacaaccacgaacagctacagcggaccattgtcaacgtcctggggccaaaggccattcttcccggcgttaccgacgtcaccgcctcctcctcctccactgccactgccggtgctgtcattgaagaaatgggcgacgtagatgccgtcgaggaggtcaagcgggcctgggaggcgctgcagaacgtcgacctgctcgatgtgacggaccagggcaaggagcggtgggcgcaggccgaaaacctgtacaatgagcgctcgacgcgagtcgaaaactccatcattgcgagactgcgagaccgcctcgccacggccaagacggccaacgaaatgtttcgcgtcttttccaagttcaatgccctgtttgtgcgccccaagattcgcggtgccattcaggagtatcagaaccagctcatggaccatgtcaagcaggccatcaacggcctgcacgagaggttcaagcagcagtatggccattccgaggcccatgccatggctcagctacgcgatctaccccctgtctcgggtgccatcatctgggcccgccagattgagcttcagctggacgggtacatgaaaaaggttgaggccgtgcttgggcccgactggactctgcatgccgagggtcacaagcttcaggaagagagcgagctgttcaagaacaaactggacacgggcaggatctacgatgcctggctggccgatgccaaccggcgaaaaatctccatcgccggccagctcttcaacatcaacagggtcaggtctgcaggtggcatcctcgagctcaacgtcaattttgatcctcagatcatcaccttgttcaaagaaaccagaaacttgacctggcagtcttatcttgttccccacgccgtcaccaccgtgtccaaggatgcaaagcgagtctacccttatgctgtcagcctgatggagggtgtgcgaaccctatcccagactctgcgccagattggtaccatgggcgaggaatcgatcctcctcaatgggtacaagaacgaggtctacaagctcatcagcgatggtatcccgctgcggtgggagtcctttgtcaactcgcacgagctcttctacgccgatcagaggcagatccgccccctgcttcccggaagcaccggctttgggcctgccaagaacacggagagcaagcatggcatgttcatctggggcttttccgcggctgtttccgtgcttcagtccaagacggctacgctcaactccatctacgctactattgagcaggctctgaaggacctcggcgcttgcccttacgatgctgccgcattccagtcccatctcgataccatccaggctgctgtcgatcagctcaacctcgagcagtatgccaacctcgatttttgggtccgcggcgtgaaccacaaggtccagaccatcctgctggagcgcctctccacggctgttcaggcttggattgccgcctttgagcaggatgtcgccgaggaggaccggcggaagctgtccaaggacgacgatggaaagcaggatggaccgaccatgaagcgcctcgtcctcgagattaccatgcgcaaccaggtcatctatctcgatccgccgctggagtacgccagggcaagctggttcctccacctccacgactggctgggcattgtctgcaacctgcgcaagatcaaggccacgcgataccagatgagcctcaacaccgatgcccaggaagaagcccgctttaccgatctgccgtctcactgtgcggatatgctccaccgcgtgtacctgtctgtcgagaagaagctcagggaagtcagcatttacgtcgacaaatggctgcagttccagtccctgtgggaccttcagtccgaccaggtctacgacatgctcggcgaccatcttcctcggtggctcgagtgtcttcaggacatccgcaaggtccgcacgacctttgacacccaagaagtcagccgctcctttggtcacatcaccatcgactacgaccaggtccagaccaaggtgaatgccaagtacgaccagtggcagcaggaaatcctcatcaagtttgccaaccggctgggcaaccgaatgcgcgacatcaacgccgacattgaaaaggcgaggaggaacctcgagggccagagctcggacacgtcgtccaccgccgcggccgtccagttcatcacggcggtgcagagctgccggcgcaacgccaagctgtgggcgcccgagattgacatgttccgccagggacagtcgaccctggtgaggcagcggtacccgttccccaacgactggctgcacattgagcagattgagagccagtgggaagctctcaaggagattctggaaaagaagtccaagatcatggaggagcagtctgacgccatgcgggcaaacattatcgcccaggacaagctgatcaatgagcgcatcggcgaggttgtggcccagtggaacgaggagaagcccgtgtcggggacgatccagcccgatgttgcctctgccactctgacgacctttgagacgcgcatcacggctctgcaggaggagttccagcaggtcatgaaggctcgcgaagcccttgatatccccggcaacaacgacagcatcttggaggttacgctggaggaagtccgcgatttccgctctgtttggtccaacctgtccaccatctggacaagtctcaacgaaacgcgcgacatgctgtggaccgccgtccagccgaggaagattcgccaaaaggtggacgacttgatcaagagcacaaaggatatgccgagtagaatgaggcagtatgctgcttttgaacacgttcagggagtgctgcgaggtttcctcaaggtcaacaacatcctgtccgacctcaagtctgacgccattcgcgaacggcactggcacaagatttacaagcagatcaagccccagaagcgcttctcgcccagctccatgacgcttggcgacgtctgggatctcaatctcaccgcgaccgaagtcattgtcaaggacattatcgcccaggcccagggcgagatggcgcttgaggagttcttgaagcaggttcgcgaaacgtggcagaactacgccctcgagatggtcaactaccagaacaaatgcaggctcattcgtggctgggacgacctcttcgccaagtgcagcgagaacctgaactcgctccaagccatgaagcactcgccttactacaaggagtttgaagaggatgcggtggcctgggaggacaagctgaaccgcgtccacgtgctctttgacgtctggatcgatgtgcagcgccagtgggtgtacctggagggcgtctttaccggaaacgcggacatcaagcatcttttgcccatagagtctggtcgtttccagaacatcaacagtgagttcttggctgtgatgaaaaaggccaataagacgccctacgtcctcgacgtgctgaacattcccaacgctcaaaagtccctggagcgtctggcggaaatgctgaacaagatccaaaaggccctgggtgaatacctggagaaggagcgcgtttctttccctcgattctactttgttggtgacgaagacttgctggagatgattggcaacagcaacgataccttgcgcatcgccaaacacttcaagaagatgtttgcgggcttggctggcctcattatggatgacgagggagtcatctctggctttacttccaaggagggcgaggccgtgacgctgaacaaggagatttcgctggccaagacgcccagaatcaacgactggcttgcgctgctggagaacggcatgaagcagacgcttgcggagcttttggccaaggccgtggacgaatacactccgattttcgagtctgacaatattgaccgggaagcgctcgttgcctttatggatgctttccccagccagattgtcgttctcgccacacaggccgcctggacgaccgccgtggattcgtcacttgcggctggcggacagacgctcaaggctctctttgatcgggaggtgcaggtgcttcgcgtccttgcggagactgtactgggagaccttgaggttattcagcgcaagaagtgcgagcagctgatcaccgagtgtgtccaccagcgcgacgtgattgagaagctggtcgacgtcaaggctgattcggccgaccactacctctggcagcttcagatgcgctacgtctacacccctgagggtaacttcctcaatcgcctgtacatcaagatggccaatgccaagctcaactacggcttcgagtacctcggcgtgcccgatcgtctcgttcggacgccccttaccgaccgctgtttcctcacgctcacccaagccctctgccagcgactcggaggctcgccctacggccccgccggcacgggcaagacggagtctgtcaaggccctgggtgtccagctcggacggtttacgctcgtcttctgctgcgacgacactttcgacttccaggccatgggacgtatcttcttgggtatctgccaggtgggtgcgtggggctgcttcgacgagtttaaccgtctcgaagagaggatcctgtctgccgtttcgcagcagattcagaacatccagctcggcctgaagcagggcgccgagaacgacaagtcgcagattgagctggttggccggcagctgcgcgtcaacgagaacacgggcatcttcatcaccatgaaccccggctatgcgggccgttccaacctgcccgacaatctgaagaagctgttccgcagcgtggccatgtccaagcccgacaaggagctcattgccgaagtcatgctgtactcgcagggcttcagccaggccaagcgactctccaagcagacggtgcccttctttgacaagtgttccaaggagctgtccaagcaggcccattacgactttggtctccgtgcgctcaagagtgtcctggtgagctctggcggtctcaagcgctctcgcttggtagacggcggcgaccttggcgccgaggagattgttgagcccgagattctcgtgcagagcatccgcgagacgattgcacccaagcttatcaagtcggacgtcgacattatgatcaacatcgaggaggactgcttccccggcgtccaatatgtgcccgccaatctgcacgctcttgaggaggccattcgcacgctcgctgccgagcgccacctcgtggtcacggatctgtggatgaccaaggttcttcagctctaccagattcaaaagatccaccacggtgtcatgatggtgggcaactctggcacgggcaagtctgccgcctggaggctcctgcttgatgcactgcaaaaggtggaaaacgtcgagggcgtttcccacgtcattgactccaaggtcatgtccaaggaggccctgtacggtaacctggactctaccactcgcgagtggacagatggtctgttcaccagcattctgcgcaagattgtggacaatcttcgtggagaagactccaagaggcactggatcgtctttgacggcgatgtcgatcccgagtgggttgaaaacttgaacagcgtgcttgacgacaacaagctgcttaccctgcccaatggagagcgtctcaacttgccggccaatgtccgcatcatgtttgaagtcgagacattgaagtatgcgacactcgctactgttagtcgatgtggtatggtctggttcagcgaggacaccgtcacgcccaacatgatggtcaccaactacatcgagacgcttcgcagcgttccctttgaggacctggacgaggatagcgttgccacaggccaaagccctgccaagacgctggccgtgcaggcccaggtggctgatctgctcaatggctacctgacggaggaagactttgtcaaccaggctttggagcgtgctctgcagtataaccacatcatggagtttacggttgcgcgagtgctcaacactctgttctctctgttgaacaaggctgtgcgcgacattatcgagtacaatggacagcacgccgacttccctctcgagtacgaccagattgaaggcttcgtggccaagaagctgctgcttgcgctggtctgggcgctcactggagactgtccgcttggagatcgaaagctgtttggcgacgacatttgcgcctttgccaactttggctcgccgcctctggatggcaccagctcgctcattgactttgacgtgctgctgccgcaggcggagtggacgccatggcagaaccaggtgcccagcatcgaggtcaacacccactccatcatccagaccgacgttgtcatcccgaccctggatactgtgcgccacgaggatgtgctttactcatggctggccgagcacaagccgctcctgctctgcggtcctcctggttcgggtaagacgatgacgctcttcagcgctcttcgcaagctgcccaacatggaggtggtcggcctcaacttctcgagcgcgacgacgcccgatctgctcatcaagacgtttgagcagtactgcgagtacaagaagacgctcaacggcgtcatgctctctcccacgcagattggccgctggctggtcatcttctgcgacgaaatcaatctcccggcgcccgacaagtacggtactcaacgcgccatttccttcctgcgccagcttgtcgagcacaacggcttctggcggacctctgacaagtcgtgggtcacgctcgaccgcatccagtttgtcggtgcttgtaacccgcctacggacgccggacgtacgcccatgggcgctcggttccttcgtcacgctccgcttgtcatggtcgactaccccggcgagctgtcgctgaaccagatttacggcaccttcaatgcggcggtgctcaagattatcccctcgctgcgtggatatgctgagccgctcacgcatgccatggtgcgcttctacctcgagtctcagcagaggttcacgcccaagattcagcctcactacgtgtacagtcctcgtgagcttacccgctgggttcgtggtgtgtatgaagcgatcaagcccctggagtcgctgtctctggaaggcctcatccgcatctgggcgcacgaggccctgcgactcttccaggatcgtcttgtgaatgaagaggagcgcaagtggaccgaagagtcggttcgtcgcatcgccattgagcatttccccaccatggacgaggaaaaggctctcggcggcccgatcctcttctccaactggctgtccaagaactacgtccccgtcgaccgggagcagctgcgagactttgtgtctgcccgcctcaagacgttctgcgaggaggaggtcgacgttccgctgatcctcttcaacgacgtcctggagcacgtgctgcgcatcgaccgtgtcttccggcagccccagggtcacttgatcctcattggtgtgagcggcggaggcaagacgacgctctcgcgcttcgtggcctggatgaacggcctcaaggtgttccagatcaaggtccacggcaagtactctgcggaagactttgacgaggacttgcgggatgtgctgcggcgatgcggctgcaagggcgagaagatttgcttcatcatggacgaggccaacgtgctggactctggtttcctggagcgcatgaacacgctgcttgccaacgccgaggtgcccggcctgttcgagggcgacgaccacgttgccctgatgacggcctgcaaagagggtgcccagcgacagaacctgcacctcgactcccccgaggagctgtacaagtggttcacccagcagattgtcaacaacctgcacgtcgtcttcaccatgaacccgcccgagggcggcctgggttccaaggccgccaccagtccggccttgttcaacagatgtgtgctgaactggttcggagattggtcggaccaggcgctgttccaggtcgggcacgagctcacgcactccatcgacctagacaagagcaacttcagcgctcccgacacgatcccggtggcttaccgcggcttgcagctgccaccgtcgcaccgcgaggcggtcgtcaactcgatggtgtacattcactattcgctgcagcggtacaacaagaagctgctgaagcagcaaggcaaggtcacgttcctgacgccccgccacttcctggactttgttacccagtatgtcaagctgtacaatgagaagcgcgaggatctcgaggagcagcagcgccacttgaacgtcggtctcgagaagctgagggacaccgtcgacaaggtccgcgacctgcgggccagtctcgcggagaagaaggcgcagctcgagcagaaggacgccgaagccaacgagaagctgcagcgcatggttgcggaccagcgcgaggcggagcagaggaagaacacttcgctggagatccaggcggcgctcgagaagcaggaggcggaggtggcgatgcgcaagaaggtcgtgctggaggacctggccaaggcggagccggccgtcgaggaggccaaggcgagcgtcagcaacatcaagcgtcagcacctggtcgaggtgcgaggcatgtccaaccccccgcagagcgtcaggctcgcgctggatgccgtctgcacgctgctcgggcacaagatcaacgactggaaggccgtccaggctgtggttcgcagggaagacttcatcgccagcatcatcatgtttgacaatgccaagatgatgaccaagggcttgcggaacaagatgcgcaacgagttcctgtcgaaccccgagttcacgtttgaaaaggtgaaccacgcttcccgggcctgcggtcccctcgtccagtgggtcattgcccaggtcagctattcggacatcctggaccgcgtcgggccgctcagggaagaggtcgcgcagctcgaggagcaggcgctcgagaccaaggccagcgcgatggcggtggagaagaacattgcggagctcgagtcgagcatcaacacgtacaagacggagtacgccacgctcatcagcgagacccaggccatcaaggcggaaatgtccaaggtgcagttcaaggtcgaccgaagcgtcaggctgctcgacagcctgtcgtccgagagggcccgctgggaggaaggaagcaagtcgtttgagacgcagatcagcacgctggtcggtgacgtcctcgtcgcttcggcctttttggcttacgccggcctgtacgaccagacgttccgcaagaacatggtggacgactggtatcaccagctggacctgtccggcatccagttcaagtcgcccaacccggtgacggagtacctgtccagcgcggacgagcgcctgggatggcaggagaacacgctgccggtggacgacttgtgcacggagaacgccatcatcctgaagcgcttcaaccggtatcccctcatcatcgacccgtccggaagggtcacggagttcttgcagagggagtgcaaggatcgtcgcctgacggtgacgagcttcttggacgacacctttacgaagcagctcgagagctccttgcgattcggcaacccgatcctgatccaagatgcggagcacctggaccccatcctcaaccacgtcctgaacaaggagtaccagcggacgggcggccgtgtcctcatccagctcggcaagcaggacattgacttttcgccgtcgttcaagctctacctctcgacgagggatccgtcggccacgtttgcgccggacatttgcagccggaccacctttgtcaacttcaccgtcacgcagagcagtctccagacgcagtcgctcaacgacgtgctcaagtcggagcggcccgacgtggacgagcggcggtccaacctcatcaagctgcagggcgagttcaaggtgcacctgcgccagctggagaagcagctgctgcaggcgctcaacgagtcgcgcggcaacatcctggacgacgacaaggtcatcgagacgctcgagacgctcaagacggaggcggcggacatctcggacaagatgcgcaacacggagggcgtcatggcggaggtggaggagattacgcagcagtacagcatcattgccaagtcgtgcagcgccgtgtttgccgtgctggagcagctgcactacatcaaccacttttaccagttctcgctgcagtactttttggacattttccagtcggtgctgaggggcaacaagaacctggtcaaggagacgaaccacaacgttcggcgcgacattatcgtcaaggacctgtttgtgaacacgttcaagcggacggcgcttgggctgctgcaaaaggaccgcatcaccctggccatgctgctcgcgcaggcgtcgccgtacaagatggacaagtcgatgattgacgtgatcctggacgaccggatcgccggcgtggacctgtcctccaacccggacgccaaggaggcggccttccaggatgcccgaaagctcagcgtgctccgggagaagctggacgccgtcccgagcggcgactgggacaggttcctcagcgaggagctggccgaggccgtggtgcccaaggtctgggacgacggcacctcggagacggaccaggccctgtactcgctcttgctggtgaagctcttccgcatggaccgcttcgtgcccgcggcggagcgctttgcggctctcgtgtttggcgccgacattttcgacattgtggaggacctcaaggagattgtgacgcaggtgtcggccacgctgcccattgcgctcgtttcgagcccgggcttcgacgccagttacaaggtggacagcctcgtgcagaggatgggcgtgcggtgcaccaacattgccatgggctcgaacgagggcctggccagcgccgacaaggccgtcagcagcgccgccgagacgggctcgtgggtgctcatcaagaacgtccacctggcgccgacgtggctgcagagcctggagaagcgcatggagtcgctcaacccgcacagcgacttccggctgttcttgtcgatggagtcgagccccaagatccccgtgaacctgctgcgcgcgtcgcgcgtgctcatgtacgagcagccggcgggcgtgcgagccaacatgaaggactcgatgtcttcgctgtcgacgcgggcggtcaagagccccgtggagaggacgaggctgtacctcttgatttcgtttttgcacgccgttgtgcaggagaggttgcggtatgcgccgaaccttggctggaagggcttctgggagtttaacgatagcgacgtaagggacccctttttcctcctctccattgttttctctcttgtttagaatagttactgactcgggttatagtacgaatgctgtgcgttcattgttgacgtctggatcgagagcgtggccggcaaccgcaccaacattgcgccgcagaacatcccctgggacatgatacggtacctggtgacggagacgtatggcggcaagattgacgacgagggcgactttagcctgctgcggcagctggtgacgtctttcctgacgccggccgcgtacgatgccgaccacaagctggtggacgggccggacggcgggctgctggtgccgtcgggcacggggctgcaggactttatggcgtgggtgcacaagctgccggagcgcgagccgccgacgtacctgggcctgccggcgaacgcggagaagctgttgcttgtgggactggggcgaagcctggttgagaatctgaagaaggttacggggctgttggatgagaatgagaggctggttaccgactcttga

SEQ ID NO: 4 - DNA, 2965 bp, Trichoderma reesei
atggcggctcatctcgacttcggcgcctctgcctcctccgcgacgccgccgcagcaacagcagcagcagcagtcgcacccgcatccgcacccgcacgccatggccgaccacaacgtctcgtcgccgcaccacccgagcaacgtcaccgtctaccagacgacggggcccgatgcccactatggccccgtcatggcccccggcgatgcgggcacgccgctgctcaacccgcgatcctgcgtcacctgccgccggcgcaaggtgcggtgcgacaagcagatgccctgctccaactgccgccgcgccctgattccctgcgtctttcccgcgcccggccgcgcgcctcgccagcaccggcccaaggaccccaacgcccctcccaaggccacgagccagcgcgaggtcgagctggtcaagcggctcaagaagctcgagggcatcgtcgaggagctgagcggccagatcgagatcgagacgggcggcagggtctcgtccgccggcgcctcgccttccaatgacggatcgccgtcgactcagcgggccaagtctaccagcctaggcgcaatgacctcgggccttgcggatcatggcgacggccacagcgccggagatgggagcgaatcgccaatgatgagggagagccggaagcagctgggccgattggtcctgaacgataacaagggcacgtctcgctatgtgagcagtgggttctggtcgaggctgaacgacgaggtacggtgcccctcctgctgtcccacccccttgctttacccccccgcgtgataccatttggtgtgtgctgacatttatgcctcctcatcaatcagttggatgccatccgagaggaaacacaaaaacttaccgacgaagacgccgaggactctgactttgaagaatcgccgcccattgactcccccttcaccgccaccagcagctccgtctatgaccaccatggcttcatcctgggatatcgatcaatcgatgtcaacatacagaaatgccatcctctgccgtcccacggcacattcctctggtcggtctacctagaaaacgtcgatccgttactcaagattctccatgtcccgaccatggaagggatactgcgggacgcccggcggaatcctgaaaagctgtccccaggtaacgaatgcctggtgtttgcggtatactttgcggccgttgttacgctcgacgatgctgatgtaggtgctcccatgcctggacattgagtttcccaggactctgctaacatcatcaggtccgaaccaactttagtattcgcaaagaggaatgccttgctcaatttcgattcgccgtggaacagtcgctcgccagggccaactttctaaacacgtccgacatcacagtcgtccaggcatttgttctattcctcctggtagtcaggcgacacgatgagtctcgcttttcctggtctctcaccgccctcgtggttcgcattgcccaaggcctgggtattcaccgtgacggaacacactttggactttctccgtacgaaacagagcagaggcggcgcctctggtgggcaatcctaactctggactttcgatcctcggaagaaatgggcaccgacttggtcgtgtccgagggcgactttgacacccaattgcctactagcatcaacgacaccgacattacccccacgggcacccaatacccagtggcaagggaaggaaagtcggacacgactatttctctcgtgaggttcgaggtctgcgccatgtcacgacgcttatccgaggcggccaacagcaagaactcatctgccaagggcgaaagcgaaggaatcgccgaaaaggagcagctgctcctcgagttctacaagaggatcgaagacaagttcctctcgcacctggacgaggacgttgacgcgctgtactgggttgcagccatgatatcacgcatcatcatggccaagatgtgcctcatcatctaccagccgatgctctttccaggaacgggcagcgagcctacggcggaaatccgagaccggatttacattgcctcgattgagattgtcgagtacaaccacaaactgaacctggaccccagaggcaagcagtatcggtggctgtttcggacctacaccaattggcccgcgattgcttatatcctggtggagacgtgccgccgtccttggtcggcccttgtccagcgcggctgggaggccgtcgtcagatacgacaaggacctcacagaaaacatgaagacggccgatcacgcctccgtgttcctcccgctgcgcaaactgttcaccagagccaagagatatcaaacaatggaggtggcgcgtctcagggccaaccctgaagaggccaggcgcctggatctcgcggagaggatcaaggcatcccaggcgcgattcgatccgatcccgggcgccgaggcgaggatgcagcaggtcagagaccgatggagaacgatggtcaatctggaaggggccatcccagcccctccaatagaacaagcacccatggtgcctcctgtatctgaagaggctcatccaccatctcagcagacacataccgaccccggacagctctaccaacagtcgcagatccagcctcaggtcagtggcgaaagttcagtccccatggacctctccaatgtcaccatggagtacatgaacgccattatggcgcaccccagcgtccccatggccgagctctggtcggtgtcgttgggagaagacccggccatgagtatggacggagggccgcagggccaggacaacatgctcggccagcagccgatgatggcgacaaacccgcagcaagccaagggcgagcatcatgttccgccctatctctggcctgagtcctttgcgacgcccagtcagaagtttgacctggaagatgcggacatgctgggggctgactttaactggcatgactggagccagagcgtccgcggcctgatggatggggggcagccgaagccggggtggtga

SEQ ID NO: 5 - DNA, 1029 bp, Trichoderma reesei
atggacgtcctcttctccatacccatgatatcctacttcctcggcccagccctcacctcctggtccacctccctcaacctcctcttcttctacatgacctggtcaaccctggtcctctcccaaccgccgctcgtcgtccacacagccggcatcctcgccatccgcatcgtcttcttcctcctcccctccctcgccaccctcctcttcgacgtctccctcccctccctcgcagaaggcatcaagcacggcggccgcagcgccctgcccccgcgcgacgccaaatccctctcccgcctcgtcggcctcgtcctcctcaacatggccatcctcaccgccgtcgaagccggcatcgacttcgccttcacccacttcttcttcaaggagcccatcttcaaaacagccaccacgttgcctctgccgtggcaaatcttcaaacacgtcctcatcctcctctccgcgcgcgaatccctcctctacgccatccaccgcttcgtcctgcacaccaagtcctcaaacgcaaccttgcgcgccctctccaaacgccatcaaaagtacgcccacgcaaaggccggagcacccttcagcctccgcctcctcgcggaccatccgcttcccttgctatgctacaagttcatcccgctcttcctccccgcggcccttctccggcctcacatcctcacttatttcctcgtcttcctgctctgcaccggcgaggaaacgctcgcaatgtcgggttacaccattgtccccggcatcatcatggggggcatcgtgcagcgcacggctatccactacgcgggcaaggggacgtccaactatggctcgtgggggatcctggactggatcaacgggacgagtcgcggtagggatgtgctggaggacgtcaagaaggaggcggataagcatcagctgcaggaacggtctgcgaagaaggtaaaccagggggctggggccgtccaggagggttttgacaacttcagggagggggtgaggacaaggagaggggggaggaagaaggcttcgcagtga

SEQ ID NO: 6 - protein, 861 aa, Trichoderma reesei
MAAEGNTNDGRSRQESVGTSSTGQQLPSLSSLFGPPSAMRPLSSPHTERNGIYPSPSPLDRPRLSSNGNLSNSYFPQTISPSPLQPRSLYDTVYNYHDRQSAHAPTSSFTGPQSPEYSNHDHRGSDARYEPEMPRKWSAYREDSRQDYQAAGSRDSSYPQGQDQLRTHLSGHKDATHDYSERRPSQVPSSDGNPPATTATTIPSDIAVSKDGLGPKIWTGTHFLPRFVRAAEVPGEGMCYFYDDGSHCKTVIDGEAVNAHWGVTKAGKPRKRLAIACVTCREKKIKCDPDYPRCVQCEKFGRICKFKNASPRAMQPSSPDRGYSKRLKLGSEAYVSNGEPPASFNRRLDYLKPRMAEQPPLPLLPEPSRIPDDVLGRAWRTDPFASNPDLITSALTRFFANVDSTMILQFLPEEAFQRWVVASVGQKSPEDLMLLYSTLAVGSALCGDPKHIAFEYAQVAHYAQRMTGVQCLQLVQSRILLAVYYISTCRTREADELISSAAASAACLQLSLELDHSRESAMTIYPLGLNRMGYAEARRRTLWSLFMLERLGGLFPERPVMTHAEDIYIRLPADLNSFEKQAESRARSFDPYCGRVVSDEPSSDATVTGYYLEMVHIWSDCQASIYRMALRRTPTEAETTKLQDLIKRAKNWAASLPPRMTFSGTNIETAAFSRSTGSFLSMHFLYHHTMIELNRHRYGAGQLPRDVQLGHSAECREHASRVLEMLNSLERILRVRSALLSIPPPAMAVAVTEAVDVLTASGPMAVLGEIIDRVRIAQSAIDKMKDVWEASSKDGLAIHRRLQKLNRIRELGSRPPSPIQGYRLLPLSDNTKDKELSHWQIFDPLEQTFPRDMDVVYVGCD

SEQ ID NO: 7 - protein, 1138 aa, Trichoderma reesei
MRLDKFNKAPFTPIKSSPVATVQPRSVPNDSPLKLSPQTTAERPDVKSEANGKPVSQTQPIPVNGSSGARRLRAGSVDKQTSSSVTPPHRRNSWFSNISAKFSGSVPQANGQTQQTGNQQHAPSQQHQPAAPTNAEPVVPKITPTKNAVLQHGLKPEGDEPYTPAPPRSGQPGLLGVFRRLSSSGGGSSGALGAAGKFNHGLVERRILNVDRDRERCPIAELKDNKLRRVSFFVDVEVAPMPKYAETEADTDSIDHTQKKKLSEKGEGEALKHPKVAEAQKEACSSSTEKTKAAPKDMTRKKEKKKRSEEERKARKEKRRRLAEESGAIPIEIHYDSSDSAELHGPPSKTNSPTINPVRIYRRCCQLRETPILKKITEQLSDTANILPNGCVNKIDLTGYFMQLQDLITLGDYLAVVSVKEVILQNSGLTDEGVRVILAGLLAAKHTEPGRRRRNSNAHHEHTSGVVERVVLKENKLGPDGWKHISLFIYKCRSLKYLDLSSIPFPRQAPANPNGTLQNGVHIPLTIADIFSKALSDRPAGPALEMLSVGETEPSMEQLGKIIDGVIKCGVKRLSTARNPLDSDGVQHVVRYLESGKCEGLDLGGIDLREHMDTIAASIKETDPLWALSISDCNLTPSSLRNILPVLAKLKNFVFIDLSHNHDLFQSTPSAVGLLRRYIPKMQCLKRLHLADVNMTAEQAIAIVEVVPEARHLCHLSLLGNTEIVALASAKTEEAQEEACALYASLMAATRLSKSLICVDIEVPSEEAGEIVKAMAKQVVAYCLQNIEHLHDVEINEAVATALAEAKGESLDSKTPTYPDVLAHLVGHDVLEEDELEEDGSGPDEDYVIGGTGVVKALACCLKNRGDESRRASGEFFRAGGVEEEHLVGPKIPTTSGKAKDLSKHLLAGARKIRQRLQPALSKARANPSDELNLRKLNFLDETLQGIIKRFEDEYPDTREHAEESQVSSQPEAEVLSSSPPPLDDAAAGGSDPEDEGELSVRPTLSRNNSILGKQLAEEEGRVHRAGHRFRSAFTEHMDLLTTIDDVEKDPKHAHVLNEIAEEVGGEFLELAKKKGAVQAFKENRDILFAALKESDPEYWERFVEAQRKARANINLPAAEKNEERLRQAQADESAIAD

SEQ ID NO: 8 - protein, 4373 aa, Trichoderma reesei
MEVTSATAPSLGAASSNGASLPAPPSPTPTPTPFPTIDPERVVEHLVAICEVALGASRSDLELPGNLLHKASYAETVSRCTRFANDSLNVLYIQKDLVQAGALENGNDAADAGEHSNGVAAPATYDYTLSTEISSSPTTVATLILLKASQAIDSSRPLTSQIFITNLPGPASLNAAAGEQGVALSPWEVLHSQVHHALVPYFDANSKSQLLANGSRGRDVDAKTGIPVTKKRLNDLELSLLHLQQNVDIPEISLTFHSLIQNVLDDAEVTGTKPSIEAIPKNLLQDSSFLNKLQANVNTWIKSIQGITKLTKETSSNAVQEFSTASQEVNFWLSMESALEGIEDQLRSEGVLLTLDILKHAKRFQATVSFVADTGLKEAKEKAQKYNQLMRDFPLDELLSAPSLLKVEEAITQIFAHLMKKLRVCPYPIRRALSLAQAISADLNDVLLRLLPGTELVNMGYPEFQNVMRTCDSIFAAWEDNIKEFTHLARMLIIRRNEKRIPINVEKNHSELESRIKYVSAFRDNHEQLQRTIVNVLGPKAILPGVTDVTASSSSTATAGAVIEEMGDVDAVEEVKRAWEALQNVDLLDVTDQGKERWAQAENLYNERSTRVENSIIARLRDRLATAKTANEMFRVFSKFNALFVRPKIRGAIQEYQNQLMDHVKQAINGLHERFKQQYGHSEAHAMAQLRDLPPVSGAIIWARQIELQLDGYMKKVEAVLGPDWTLHAEGHKLQEESELFKNKLDTGRIYDAWLADANRRKISIAGQLFNINRVRSAGGILELNVNFDPQIITLFKETRNLTWQSYLVPHAVTTVSKDAKRVYPYAVSLMEGVRTLSQTLRQIGTMGEESILLNGYKNEVYKLISDGIPLRWESFVNSHELFYADQRQIRPLLPGSTGFGPAKNTESKHGMFIWGFSAAVSVLQSKTATLNSIYATIEQALKDLGACPYDAAAFQSHLDTIQAAVDQLNLEQYANLDFWVRGVNHKVQTILLERLSTAVQAWIAAFEQDVAEEDRRKLSKDDDGKQDGPTMKRLVLEITMRNQVIYLDPPLEYARASWFLHLHDWLGIVCNLRKIKATRYQMSLNTDAQEEARFTDLPSHCADMLHRVYLSVEKKLREVSIYVDKWLQFQSLWDLQSDQVYDMLGDHLPRWLECLQDIRKVRTTFDTQEVSRSFGHITIDYDQVQTKVNAKYDQWQQEILIKFANRLGNRMRDINADIEKARRNLEGQSSDTSSTAAAVQFITAVQSCRRNAKLWAPEIDMFRQGQSTLVRQRYPFPNDWLHIEQIESQWEALKEILEKKSKIMEEQSDAMRANIIAQDKLINERIGEVVAQWNEEKPVSGTIQPDVASATLTTFETRITALQEEFQQVMKAREALDIPGNNDSILEVTLEEVRDFRSVWSNLSTIWTSLNETRDMLWTAVQPRKIRQKVDDLIKSTKDMPSRMRQYAAFEHVQGVLRGFLKVNNILSDLKSDAIRERHWHKIYKQIKPQKRFSPSSMTLGDVWDLNLTATEVIVKDIIAQAQGEMALEEFLKQVRETWQNYALEMVNYQNKCRLIRGWDDLFAKCSENLNSLQAMKHSPYYKEFEEDAVAWEDKLNRVHVLFDVWIDVQRQWVYLEGVFTGNADIKHLLPIESGRFQNINSEFLAVMKKANKTPYVLDVLNIPNAQKSLERLAEMLNKIQKALGEYLEKERVSFPRFYFVGDEDLLEMIGNSNDTLRIAKHFKKMFAGLAGLIMDDEGVISGFTSKEGEAVTLNKEISLAKTPRINDWLALLENGMKQTLAELLAKAVDEYTPIFESDNIDREALVAFMDAFPSQIVVLATQAAWTTAVDSSLAAGGQTLKALFDREVQVLRVLAETVLGDLEVIQRKKCEQLITECVHQRDVIEKLVDVKADSADHYLWQLQMRYVYTPEGNFLNRLYIKMANAKLNYGFEYLGVPDRLVRTPLTDRCFLTLTQALCQRLGGSPYGPAGTGKTESVKALGVQLGRFTLVFCCDDTFDFQAMGRIFLGICQVGAWGCFDEFNRLEERILSAVSQQIQNIQLGLKQGAENDKSQIELVGRQLRVNENTGIFITMNPGYAGRSNLPDNLKKLFRSVAMSKPDKELIAEVMLYSQGFSQAKRLSKQTVPFFDKCSKELSKQAHYDFGLRALKSVLVSSGGLKRSRLVDGGDLGAEEIVEPEILVQSIRETIAPKLIKSDVDIMINIEEDCFPGVQYVPANLHALEEAIRTLAAERHLVVTDLWMTKVLQLYQIQKIHHGVMMVGNSGTGKSAAWRLLLDALQKVENVEGVSHVIDSKVMSKEALYGNLDSTTREWTDGLFTSILRKIVDNLRGEDSKRHWIVFDGDVDPEWVENLNSVLDDNKLLTLPNGERLNLPANVRIMFEVETLKYATLATVSRCGMVWFSEDTVTPNMMVTNYIETLRSVPFEDLDEDSVATGQSPAKTLAVQAQVADLLNGYLTEEDFVNQALERALQYNHIMEFTVARVLNTLFSLLNKAVRDIIEYNGQHADFPLEYDQIEGFVAKKLLLALVWALTGDCPLGDRKLFGDDICAFANFGSPPLDGTSSLIDFDVLLPQAEWTPWQNQVPSIEVNTHSIIQTDVVIPTLDTVRHEDVLYSWLAEHKPLLLCGPPGSGKTMTLFSALRKLPNMEVVGLNFSSATTPDLLIKTFEQYCEYKKTLNGVMLSPTQIGRWLVIFCDEINLPAPDKYGTQRAISFLRQLVEHNGFWRTSDKSWVTLDRIQFVGACNPPTDAGRTPMGARFLRHAPLVMVDYPGELSLNQIYGTFNAAVLKIIPSLRGYAEPLTHAMVRFYLESQQRFTPKIQPHYVYSPRELTRWVRGVYEAIKPLESLSLEGLIRIWAHEALRLFQDRLVNEEERKWTEESVRRIAIEHFPTMDEEKALGGPILFSNWLSKNYVPVDREQLRDFVSARLKTFCEEEVDVPLILFNDVLEHVLRIDRVFRQPQGHLILIGVSGGGKTTLSRFVAWMNGLKVFQIKVHGKYSAEDFDEDLRDVLRRCGCKGEKICFIMDEANVLDSGFLERMNTLLANAEVPGLFEGDDHVALMTACKEGAQRQNLHLDSPEELYKWFTQQIVNNLHVVFTMNPPEGGLGSKAATSPALFNRCVLNWFGDWSDQALFQVGHELTHSIDLDKSNFSAPDTIPVAYRGLQLPPSHREAVVNSMVYIHYSLQRYNKKLLKQQGKVTFLTPRHFLDFVTQYVKLYNEKREDLEEQQRHLNVGLEKLRDTVDKVRDLRASLAEKKAQLEQKDAEANEKLQRMVADQREAEQRKNTSLEIQAALEKQEAEVAMRKKVVLEDLAKAEPAVEEAKASVSNIKRQHLVEVRGMSNPPQSVRLALDAVCTLLGHKINDWKAVQAVVRREDFIASIIMFDNAKMMTKGLRNKMRNEFLSNPEFTFEKVNHASRACGPLVQWVIAQVSYSDILDRVGPLREEVAQLEEQALETKASAMAVEKNIAELESSINTYKTEYATLISETQAIKAEMSKVQFKVDRSVRLLDSLSSERARWEEGSKSFETQISTLVGDVLVASAFLAYAGLYDQTFRKNMVDDWYHQLDLSGIQFKSPNPVTEYLSSADERLGWQENTLPVDDLCTENAIILKRFNRYPLIIDPSGRVTEFLQRECKDRRLTVTSFLDDTFTKQLESSLRFGNPILIQDAEHLDPILNHVLNKEYQRTGGRVLIQLGKQDIDFSPSFKLYLSTRDPSATFAPDICSRTTFVNFTVTQSSLQTQSLNDVLKSERPDVDERRSNLIKLQGEFKVHLRQLEKQLLQALNESRGNILDDDKVIETLETLKTEAADISDKMRNTEGVMAEVEEITQQYSIIAKSCSAVFAVLEQLHYINHFYQFSLQYFLDIFQSVLRGNKNLVKETNHNVRRDIIVKDLFVNTFKRTALGLLQKDRITLAMLLAQASPYKMDKSMIDVILDDRIAGVDLSSNPDAKEAAFQDARKLSVLREKLDAVPSGDWDRFLSEELAEAVVPKVWDDGTSETDQALYSLLLVKLFRMDRFVPAAERFAALVFGADIFDIVEDLKEIVTQVSATLPIALVSSPGFDASYKVDSLVQRMGVRCTNIAMGSNEGLASADKAVSSAAETGSWVLIKNVHLAPTWLQSLEKRMESLNPHSDFRLFLSMESSPKIPVNLLRASRVLMYEQPAGVRANMKDSMSSLSTRAVKSPVERTRLYLLISFLHAVVQERLRYAPNLGWKGFWEFNDSDYECCAFIVDVWIESVAGNRTNIAPQNIPWDMIRYLVTETYGGKIDDEGDFSLLRQLVTSFLTPAAYDADHKLVDGPDGGLLVPSGTGLQDFMAWVHKLPEREPPTYLGLPANAEKLLLVGLGRSLVENLKKVTGLLDENERLVTDS

SEQ ID NO: 9 - protein, 937 aa, Trichoderma reesei
MAAHLDFGASASSATPPQQQQQQQSHPHPHPHAMADHNVSSPHHPSNVTVYQTTGPDAHYGPVMAPGDAGTPLLNPRSCVTCRRRKVRCDKQMPCSNCRRALIPCVFPAPGRAPRQHRPKDPNAPPKATSQREVELVKRLKKLEGIVEELSGQIEIETGGRVSSAGASPSNDGSPSTQRAKSTSLGAMTSGLADHGDGHSAGDGSESPMMRESRKQLGRLVLNDNKGTSRYVSSGFWSRLNDELDAIREETQKLTDEDAEDSDFEESPPIDSPFTATSSSVYDHHGFILGYRSIDVNIQKCHPLPSHGTFLWSVYLENVDPLLKILHVPTMEGILRDARRNPEKLSPGNECLVFAVYFAAVVTLDDADVRTNFSIRKEECLAQFRFAVEQSLARANFLNTSDITVVQAFVLFLLVVRRHDESRFSWSLTALVVRIAQGLGIHRDGTHFGLSPYETEQRRRLWWAILTLDFRSSEEMGTDLVVSEGDFDTQLPTSINDTDITPTGTQYPVAREGKSDTTISLVRFEVCAMSRRLSEAANSKNSSAKGESEGIAEKEQLLLEFYKRIEDKFLSHLDEDVDALYWVAAMISRIIMAKMCLIIYQPMLFPGTGSEPTAEIRDRIYIASIEIVEYNHKLNLDPRGKQYRWLFRTYTNWPAIAYILVETCRRPWSALVQRGWEAVVRYDKDLTENMKTADHASVFLPLRKLFTRAKRYQTMEVARLRANPEEARRLDLAERIKASQARFDPIPGAEARMQQVRDRWRTMVNLEGAIPAPPIEQAPMVPPVSEEAHPPSQQTHTDPGQLYQQSQIQPQVSGESSVPMDLSNVTMEYMNAIMAHPSVPMAELWSVSLGEDPAMSMDGGPQGQDNMLGQQPMMATNPQQAKGEHHVPPYLWPESFATPSQKFDLEDADMLGADFNWHDWSQSVRGLMDGGQPKPGW

SEQ ID NO: 10 - protein, 342 aa, Trichoderma reesei
MDVLFSIPMISYFLGPALTSWSTSLNLLFFYMTWSTLVLSQPPLVVHTAGILAIRIVFFLLPSLATLLFDVSLPSLAEGIKHGGRSALPPRDAKSLSRLVGLVLLNMAILTAVEAGIDFAFTHFFFKEPIFKTATTLPLPWQIFKHVLILLSARESLLYAIHRFVLHTKSSNATLRALSKRHQKYAHAKAGAPFSLRLLADHPLPLLCYKFIPLFLPAALLRPHILTYFLVFLLCTGEETLAMSGYTIVPGIIMGGIVQRTAIHYAGKGTSNYGSWGILDWINGTSRGRDVLEDVKKEADKHQLQERSAKKVNQGAGAVQEGFDNFREGVRTRRGGRKKASQ

SEQ ID NO: 11 - protein, 859 aa, Trichoderma reesei
MPHRERGKQREGGDSYRPSRPARSRSRSRSPPRAPVPVRTEEEKQAAAKAEYEKLLNMRSGGTYIPPARLRALQAQITDKSSKEYQRMAWEALKKSINGLINKVNTANIKHIVPELFGENLVRGRGLFCRSIMKAQAASLPFTPIYAAMAAIVNTKLPQVGELLVKRLIMQFRKGFKRNDKAVCLSSTTFLAHLINQQVQHEMLAGQILLLLLHKPTDDSVEIAVGFCKEVGQYLEEMQPAISMAVFDQFRNILHESDIDKRTQYMIEVLFQIRKDKFKDHPAIKEELDLVEEEDQITHKVELDGEIDVQDGLNIFKYDPEWEEHEEAYKRLKAEILGEASDDEEGDEDEDEDESSEDEENEETKAMEIKDQSNADLVNLRRTIYLTIMSSADPEEAVHKLMKINLPVGQEPELPSMIVECCSQEKTYTKFFGLIGERFAKINRLWCDLFEQAFVKYYETIHRYENNKLRNIAMLFGHMFASDALGWHCLSVIHLNEEETTSSSRIFIKILFQHISEEIGLAKLRARMTDETLRPSLEGLFPRENPRNIRFSINYFTSIGMGVLTEEMREHLMNMPKPALPAPAAQDRSDTDSVSSYSSYTHSSYSSRSRSRSRSVGRRSGGRGRSLSRTPPRRGARSRSYSDDSRSPSRSRSRSRSDSVSTRGRRRASYSASPPRRGGRRVASRSRSYSSGSSRSPPPRNRGRARSNSYSSYSRSPSSSPRRGRDADSASPPPRRGRPRQSPPGGPAGRRNSSSVGSGGPRKKPRRDSRSPSRDYSSRSPSRSPSRSRSPPPAARGRRGSYTPSRSRSPPPRRVRDGSPGRLRGGRSPSPPLPVKRRRYDSESVSRSPPPLKRGRRDN

SEQ ID NO: 12 - DNA, 2580 bp, Trichoderma reesei
atgccgcaccgcgagcgcggcaagcagcgagaaggcggcgactcgtaccgcccctcgaggccagcgcgttcacgctcgcgctcgcgatcgccgcctcgcgcgccggtgcccgtgcggacggaggaggagaagcaggcggcggcaaaggccgagtacgagaagctgctcaacatgcggtcgggcggcacgtacatcccgccggcgaggctgagggcgctgcaggcgcagatcacggacaagagcagcaaggagtaccagcggatggcgtgggaggcgctcaagaagagcatcaacggcctgatcaacaaggtcaacacggccaacatcaagcacattgtgcccgagctgtttggcgagaacctggtgcgcggccgcggcctcttctgccgctccatcatgaaggcccaggccgccagtttgcccttcacgcccatctacgccgccatggccgccattgtcaacaccaagctgccgcaggtcggcgagctgctggtcaagcgcctcatcatgcagttccgcaagggcttcaagcgcaacgacaaggccgtctgtctgtcgtcgaccaccttcctcgcccacctcatcaaccagcaggtgcagcacgagatgctggccggccagatcctgctgctgctgctgcacaagccgaccgacgacagcgtcgagattgccgtgggcttctgcaaggaggttggccagtacctcgaggagatgcagcctgccatttccatggccgtcttcgaccagttccgcaacatcctccacgagtccgacattgacaagcgaacgcagtacatgattgaggtgctcttccagatcaggaaggacaagttcaaggatcacccggccatcaaggaggagctggacttggtggaggaggaggaccagatcacgcacaaggtggagcttgatggcgagattgatgtgcaggacggactcaacatcttcaagtacgacccggagtgggaggagcatgaggaggcatacaagaggctcaaggcggagattctgggcgaagccagcgatgacgaggagggcgacgaggacgaggacgaggacgagagctccgaagatgaagaaaacgaagagacaaaggccatggagatcaaggaccagtctaacgccgacttggtcaacctacggaggaccatctacctcaccatcatgtcgagcgccgacccagaggaagcagttcacaagctgatgaagatcaacctgcccgtcggccaggaacccgagctgccctcgatgattgtcgagtgttgctcgcaggagaagacgtacaccaagttctttggcttgatcggcgagcgtttcgccaagatcaatcggctgtggtgcgacctctttgagcaggcctttgtcaagtactacgagacgatccaccgatacgaaaacaacaagctgcggaacattgccatgctgtttggccacatgtttgcttccgacgctctgggctggcactgcctttccgtcattcacctcaacgaggaggagaccacgtcgagcagccgcatcttcatcaagattctgttccagcacatttccgaggaaatcggcctggctaagctccgggcacgcatgactgacgagacgctgcggcccagcctcgaaggcctcttccccagagagaaccctcgcaacatccgattctccatcaactacttcaccagcatcggcatgggtgtactgaccgaggagatgcgagagcacctcatgaacatgcccaagcctgcgctgcccgcccctgctgctcaggaccgctcggatacggactccgtctcgagctattcgtcttacactcactcatcatactcttcccgctcgcgctcacggtcccgatctgtgggtcgtcggagcggcggtcgaggccgatcgctttcccgaactccgcctcgacgtggcgcaaggagccgatcctactctgacgactcacggtcaccgtcgcggtcaagatcacgatcccgctccgattccgtctctactcgtgggcgaaggcgagcgtcgtactcggccagtcctccccggcgtggtggccgtcgggttgccagcagaagccgaagctactcgtcgggctcctcacggtctccgccaccacggaaccgcggtcgcgcacgaagcaactcgtatagttcctacagccgctctccatcttcttcaccacgacgcggcagagacgcagactcggccagcccgcctccgcgaaggggtcgaccgcgccagagcccaccaggcggtcccgcaggtcgaaggaacagctcgtctgtcggcagcggagggccccgcaagaagccccgacgggacagccgatcgccgtctcgcgactattcgtcccggtccccgtctcggtcgccgtcgagatctcgatcgcctccgccggctgcgcgtggccgaaggggctcttatacgccgtcacgcagccgcagcccgcctccgcgcagggtgagggatggctcgccgggtcgtctgaggggtgggaggtcgcctagtcctcctttgccggtgaagaggaggcggtatgatagcgagagtgtttctcggtcgccgcctcctttgaagcgcgggagaagggataactaa

SEQ ID NO: 13 - protein, 543 aa, Trichoderma reesei
MTVLTSPLASYNVANKLYKTTLLNTVCLVAGLSIFFFGYDQGLMGGVNTTRDYAERMGFGHWDEDQNIVVVDKPLLQGGIVAVYYLPGTLCGCLLGGWLGDRYGRIKTIAIACAWSVCAAALQASAMNANWMFCARVLNGVGTGILNAITPVWATETAAHTSRGQFVSIEFTLNILGVVVAYWLEFGTSKYHDNTSSFIWRFPVAFQILPLILLFLIIWIMPESPRWLVKVGREEEARFILGRLRGNEGEDGLKAEAEYNDIVNIHKLEVDTAKQQSYFSMFFGIGSGKLHTGRRVQLVIWLQILQEWIGIAGITIYGPEIFTIAGISAKDRLWVSGINNITYMFATLICVFTIDRIGRRWTLYWGAVGQGICMFVAGGLARATINASGKASQSHIGGAATFFVFLYTAIFGATWLTVPWLYPAEIFPLQVRAKGNAWGVVGWSIGNGWCVLLLPTIFKALNEKTLYIFGAVNALSILVVWALYPESNQRTLEEMDLVFASDSIWAWEAERNFAKLKAENPDLVQGSTNHGVVDIEQVAEPKE

SEQ ID NO: 14 - DNA, 1870 bp, Trichoderma reesei
atgaccgtcctcacctcacctctggccagctataatgtggccaacaagctgtacaaaaccactctgctcaacaccgtctgcctcgtggccggactgtcgatcttcttcttcggctatgatcagggattgatgggcggtgttaacacgacgcgcgactatgccgagcgcatgggctttggccactgggacgaagaccagaacattgtcgtcgtcgataagccgctgctgcagggcggtatcgtagctgtctactatctccccggaacgctgtgcggttgtctgcttggcggttggcttggtgatcgctatggccgtatcaaaacaattgccattgcctgtgcgtggagtgtctgcgcagccgccctgcaggcctcagctatgaatgcgaactggatgttttgcggtatgtcgatgattcttggacaatcacaaccgaactattactgatgatgagatgaaacagcccgcgttctgaacggcgtcggcactggaatcttgaacgcaatcacgcctgtgtgggcaaccgagactgctgctcacacttctcgaggccagttcgtttccattgagttcaccctcaacattcttggtgttgttgtagcctactggctggaattgtacgtgcctcctcactcaggatccccagtcttgtggaaagtctccctaatgcggtggcagtggtacttctaaatatcacgacaacacatcctccttcatctggagattcccggtcgccttccagatcctccccctaatccttctgttcctcatcatctggatcatgcctgaatccccccgctggctcgtcaaagtgggtcgtgaagaagaggctcgcttcatccttggtcgtctccgtggcaatgagggcgaggacggcctcaaggcggaagcagagtacaatgatattgtcaacatccacaagcttgaagtagacaccgccaagcagcagagctacttctccatgttctttggcattgggtctggaaagctacacactggccggcgcgtgcagctggtcatctggctccagatattgcaagagtggatcggtattgcgggaatcaccatttacggccctgagatctttacgattgctggcatcagcgcaaaggacagactctgggttagcgggatcaacaatatcacatacatggtacgtttagccaacacctcctcacctcaaagattccatcacactaacacgggagcagttcgccacactgatctgcgtcttcaccatcgatcgcataggtcgccgttggactctgtactggggagctgtcggccagggcatttgcatgttcgtcgccggtggcctcgctcgcgcaaccatcaatgcctcaggcaaagcaagccagagccacatcggcggcgctgcaacattctttgtgttcctctacactgccattttcggcgctacctggctgacggttccttggttgtatccggccgagattttccctctgcaggttagagccaagggaaatgcctggggtgtcgttggctggtccattggcaacggctggtgtgtaagtgcacttttcattctcctctcccgtctgggctcttctggtctaatcttctctaggtgctcctgcttcctacgatcttcaaggcgctcaacgaaaagacactctacatttttggcgccgtcaacgccctgtccatcctcgtcgtgtgggctctgtaccccgaatcgaatcaacgaactctagaggagatggacctcgtctttgctagcgacagcatctgggcctgggaggctgagcgtaattttgccaagctcaaggctgaaaacccggatcttgttcagggctcaacaaaccacggagttgtagatattgagcaagttgccgagccaaaggagtag

SEQ ID NO: 15 - protein, 735 aa, Trichoderma reesei
MAVNRIRGAFAAPRKGETFELRAGLVSQYAYERKESIQKTIMAMTLGKDVSALFPDVLKNIATSDLDQKKLVYLYLMNYAKTHPDLCILAVNTFVQDSEDPNPLVRALAIRTMGCIRVDKMVDYMEEPLRKTLRDESPYVRKTAAICVAKLFDLNPAMCIENGFIETLQEMIGDPNPMVVANSVQALAEISETAPETRALLVTPPVLKKLLMAMNECTEWGRITILTVLADYAATDVKESEHICERVIPQFQHVNPSVVLAAVKVVFIHMKSINPELVRSYLKKMAPPLVTLVASAPEVQYVALRNIDLLLQAKPDILSKELRVFFCKYNDPPYVKMQKLEIMVRIANEKNYEQLLSELKEYALEVDMDFVRRAIKAIGQVAIKIEEASGKCVQALEDLLATKVNYVVQEVVVVIKDILRKYPGYEGVIPSLCNYIDELDEANARGSLIWIVGEYAEKISNAEEILEGFVDTFLEEFTQTQLQILTAVVKLFLKKPSGAQGLVQKVLQEATTNNDNPDIRDRAYVYWRLLSGDLEVAKNIVLSQKPTISTTMTSLPTALLEQLLSELSTLASVYHKPPEAFVGKGRFGADEIQRAAIQEQRQNAAENPIAASVAAAAANGSSSVSQNNIENLLDIDFDGAAPASQEQNSAAGTPDRVSSPATGGMADMMSMFDAPPAGSSGGAPSGGMNDLMNGFEGLNFGATSTNQPLPAAMQLHNAQGGSQPKKDSDDLLGLL

SEQ ID NO: 16 - DNA, 2589 bp, Trichoderma reesei
atggcggtgaatcgcatccggggcgcctttgccgcgcctcggaagggagagacattcgagctgcgggccggcctggtgtcgcagtatgcctacgagcggaaggagtccatccagaagaccatcatggccatgacgctgggcaaggacgtgtccgccctgttcccagacgtcttgaagaacattgccacgtccgacctggaccagaagaagctggtctacctctacctcatgtatgtggctgcagacaatggccgaccatgatcacacacacggagcgaaggacgagatactgcctgacgtggcgatgcggtgctaacgtggagtgtgaccccaggaactacgcaaagacacacccagacctctgcattctcgccgtcaacacgttcgtgcaagactcggaagacccgaacccgctggtgcgagcgctggccatccgcacaatgggctgcatccgggtggacaagatggtcgactacatggaggagccgctgcggaagacgctgcgggacgagtcgccgtacgtgcgcaagacggccgccatctgcgtggccaagctgttcgacctgaacccggccatgtgcatcgagaacggcttcatcgagacgctgcaggagatgattggcgacccgaaccccatggtggtcgcaaactcggtccaggcgctggccgagattagcgagacggcgcccgagacgcgggcgctgctggtgacgcccccggtgctcaagaagctgcttatggccatgaacgaatgcaccgaatggggtagaatcaccattctgaccgtgctggcagactacgctgccaccgacgtcaaggagtcggagcacatctgcgagagggtcattccgcagttccagcacgtcaaccctagcgtggtcctggctgctgtcaaggtggtctttattcatatgaagtcgattaacccggagctcgtgcggtcatatcttaagaagatggcgcctccactcggtgcgttccgatcatgtccccgatttgacatctgagaagacatgacgtgactatgctaacactgcagcttgtatacagtcacactggttgcttctgcccccgaggtccaatacgtcgctctcagaaacattgatctgctccttcaagccaagcccgacatcctgagcaaagagttaagagtcttcttttgcaaatacaacgacccgccgtacgtcaagatgcaaaagctggaaatcatggtcaggatagcaaacgaaaagaactacgagcagctcctgtctgagctcaaggaatacgccctggaagtggacatggactttgtgcgccgagccatcaaggccatcggccaggtggccatcaagattgaggaggccagtggcaagtgcgtgcaggcgctggaagatcttctcgctaccaaggtcaactacgtggtgcaagaggttgtcgtggtcatcaaagatatcctgcgaaagtaccccggttacgagggcgtgatcccctcgctctgcaactacattgacgagctcgacgaggccaatgctcgtggatccctcatctggattgtgggagagtacgccgagaagattagcaacgctgaggagattctggagggttttgtagacacctttttggaggagttcactcaggtatgtggagagctgtggaaaagtcggggattttggctaatcgaactgcagacacaactccagatccttacagctgttgttaagctgtttttgaagaagccgagtggcgcgcagggcctggttcagaaggtgctgcaggaggcaacaaccaacaacgacaaccccgatatccgcgacagagcatacgtctactggcgattgttatcgggagatttggaggtggccaaggtaggagtcgttggcgtcctttgatgagagctgcgcatactgacggatctcaagaacattgtcctgtcacagaagccgaccatttcaacaacaatgacaagcctgccgactgcgctactggagcagctgctgtcggagctgtcaactctggcgtcggtataccacaagcccccggaagcctttgtcggcaagggccggttcggtgccgacgagatccagcgagccgccatccaggagcagcgccagaacgccgcggaaaaccccatcgccgcatccgtggctgccgccgccgccaatggctcctcgtcggtctcgcaaaacaacattgagaacctgctggacattgactttgacggcgcagcaccggcctctcaggagcagaacagcgcggcgggaacacctgaccgggtgtcgagcccggccacgggtggcatggccgacatgatgagcatgtttgatgcgcctccggctggcagctctggaggtgctccgtccggcggcatgaacgacttgatgaacggatttgaggggctcaactttggggccacgagtacaaatcagccgttgccggcggcgatgcagctgcacaatgcgcaaggcggctctcagccgaagaaggatagcgatgatcttttgggtttgttgtaaatgttggaggagcgtatatgcatgcaagcagcaagccagaaggggagaagaatcgacaagagagactggaggaggaggcaagggaggggggggg

SEQ ID NO: 17 - protein, 553 aa, Trichoderma reesei
PTISMSPPSPVDHPMEGTSPEDNSPTGHGHGSGSDPEGSSNGKPPVSRAGNASPQEHGALTTSSNMPAPPPAAAAAVHQPKIVQTAFIHKLYNMLEDTSIQHLISWSSSAESFVMSPSADFSKVLSQYFKHTNISSFVRQLNMYGFHKERDVFHTGNPETTLWEFKHGNGNFKRGDLVGLREIKRRASRHALVNRENTFPKTSTSQPGTPIEPVQVPPDSIEARIANLEHSLYDTAARLQRSEESAHYMHVKNQAIMETLNRLLFFNQELSKGILSLVPPDNPVHRDVMTLQGEMVRQAEMLRSLDEPHEPVYSARQQFFGTVDNAPVSPRQLPQDDNRRVTLNVPQGRSQPSYRPAVPSNLSAGTRRPYGSISGGAGSSPLRNAAPAPPAGPHPLSNVETVPSNLARRHTAADIRAHGWQPNAPPYPPGAVPPPVWPQSPNRPEDQRIRDSLSSFSLQAPSAHGHPHSRPATPPHAPFSNGASGGSDTFGSWSWGAASNRESKTIGALKESSAPPTRRGSMAHILNPSDTAERSDEDEDPRGDDDRKRKRRQ

SEQ ID NO: 18 - DNA, 1975 bp, Trichoderma reesei
cctaccatcagcatgtctccaccgagtcccgtcgaccatccgatggagggcacgtcgccagaggacaactcgcccacgggccacggccacggctccggctccgaccccgaaggcagctccaatggcaagccgcccgtctcccgggccggcaacgcatcgccgcaggagcacggcgccctcaccacctcgagcaacatgcccgcccctcctcccgccgccgccgctgctgttcaccagcccaagatcgtccagactgcctttatccataagctatacaagtgaggaccctggcctccctctctcccccaaacccccctttcccccttcaatgcttccttgacctgccaaggagggccccgagactaaagcgacagctgctaacggcgcaaatagcatgttggaagacaccagtatacagcatctcatatcctggtcatcgtccgccgaaagcttcgtcatgtcgccctctgccgacttctccaaggtattatcgtaagcagtgccccgacaatcctcgcccttttctggccaattcgctcattctcgcccccagacaatacttcaaacacaccaacatatcgtctttcgtgcgtcagctcaacatgtacggattccataaaggtacctcctgcccccgcttgcttctgcctcgaacctccgaacctcgaatggcttaccatggatgcagaacgagacgtgtttcataccggcaaccccgaaacgacgctctgggaattcaagcacggcaacggcaacttcaagcgcggcgacctcgtcggccttcgagagatcaagcgtcgcgccagccgacacgctctcgtcaaccgggaaaacacctttcccaagacctccacatctcagcctggcacgcccattgagcctgttcaagtcccgccggatagcatcgaggctagaatagccaacctggagcactccctgtacgatacggctgcgagactgcagagaagcgaggaatcggcccactacatgcatgtcaagaaccaggccatcatggagacactcaaccgactgctcttcttcaaccaagagttgtccaaggggatactgtcgcttgtacctccagacaaccctgtacacagagatggtttgtcacacgaaacaatcaggacgatacgctattcatgggagacgatgaaactgacaatgagggcttcaagtcatgacactccagggcgaaatggtgaggcaggcagaaatgctgcgctcgctcgacgagccgcacgagcctgtctattccgccaggcaacagttcttcggcacggtcgacaacgcccccgtgtccccgcggcagcttcctcaagacgacaacaggcgagtcacgctcaacgttccgcagggccggagtcagccgtcgtaccgaccagccgtcccctcgaacctgtccgccggcacgcggcgaccctacggatccatcagcggcggtgccggctcctcacctctgcgcaatgccgcgccggcacccccagcagggccgcatcccctttccaacgtggagaccgtccccagcaacctggccagacgccataccgccgcagacattcgcgcacacggatggcagcccaacgcaccgccttatccccctggagccgtgccgccgcccgtatggcctcaatctcccaatcggcccgaggatcagaggataagagactctctctcttcgttttccctgcaggccccatcggctcatggacacccgcactcgcggccggcgacgccaccccacgctcccttttcaaacggcgcgagcggagggtcggatacctttggcagctggtcgtggggcgcggcatcgaaccgcgagagcaagacgattggagcgttgaaggagtcgtcagcgccgcccaccaggaggggcagcatggcacacatcctcaaccccagcgataccgctgagcgatcagacgaggacgaggacccccggggcgacgacgacagaaagcgaaaacgaagacag

SEQ ID NO: 19 - protein, 609 aa, Trichoderma reesei
MGQAPSQTRRTRRTHEELTQELAYRFKEKCFTSLEYYSLKDVFKKLADQQGDIRYLKEDTIARFLEIPDILGASPVIFHMISYLGAFPFLQEAPVVLELAQLIMVVVIMTERYKRVLAKGSTDRTKLFFKSLAVYDRKVMEETGSSPRNSTSKDATARPSANTRGFAIDEPMAEDEDDGDDDDDDLVITAFELLDIDEATKHGEAAAIKEAIIPTDNFRKLIMLLLLIAPLDAQESLSQYSSRVAGPELESLRATAECVLASFVDVETSPGIGYTRFKTVIPVLFPNLFAGFNGLFEHFLFSKDLDFSKHKVEKPGDEQLIIGKIAQPLLPTPGDIMTEHTLSQLSLFLPGSSLFRRVRLLYSGNDAGFSMGSLQTKVFNWRAPTILLVSGSRLADVPEGGQEASFASSLPTKRFPHGSKSERVTFGVYVREPWKHTHKECFGNSETILFQLEPIHDVFPASTINTDYVTFTKPPGNRPCLAFGCPHPKPTQSHRKEGIHALGAVSLLFDESFEFGVFNHDYKSRGGAFHTSIVRKYDFQDRFRIENMEVWGCGGDEEAKAQAERWAWEEREAEARRRINLGTGDIEADRALLEMAGLVGGNRSGGSMG

SEQ ID NO: 20 - DNA, 1900 bp, Trichoderma reesei
atgggccaagccccttctcagacgcggcggactcggcggacgcacgaggaactgacacaggagcttgtgagcgcgccgttgcaccttggcctgggaacaagcaagggcctaggctttgctaactgtgcgtgaacaggcgtacagattcaaggaaaagtgcttcacgtcgctggagtactactcactgaaggatgtcttcaagaaactggccgaccagcagggcgacatacggtatctcaaggaggacaccatagctcgcttcctcgagatcccagacatcctcggggcttcgccggtcatcttccacatgatctcgtatctgggtgcgtttccctttctgcaggaggcccccgtggtgctcgagctggcgcagttgatcatggtcgttgtcatcatgacggagcgatataagcgcgttcttgcgaagggctcgacagacaggacgaaattattttttaaaagcttggctgtgtatgaccggaaagtgatggaggagaccggttcttcaccgcggaattccacttcaaaagacgccactgcgaggccgagcgccaacacaagaggctttgcaatcgacgagccgatggccgaggacgaggatgatggcgacgacgacgacgatgatcttgtcattacggccttcgagctgctcgacatcgatgaagctaccaagcacggagaagccgctgccattaaagaagccatcatcccgaccgacaacttccgcaaacttattatgctgctgctgctgattgcgccactggatgctcaggagagtctatcgcaatattccagccgggtcgcgggccccgaactagagtcgctgcgagccacggcagagtgtgtactagcatccttcgtcgatgtcgagacgtcgccaggcattggatatacccggttcaagactgtcattccggtcctattccccaacctctttgctgggttcaatggcctgtttgaacactttcttttctccaaggatctggacttctcgaagcacaaggttgagaagcctggtgacgagcagctcatcattggcaagattgcgcaacctctgctcccaacccctggcgacatcatgactgagcatacgctgtcgcaactttcactgtttctacctggctcctctctgttccgaagagtgagattgctctattcgggaaacgatgctggattctccatgggcagcctccagaccaaggtctttaactggagagccccaaccattcttctagtcagcggatcgagattagcagacgtccccgagggaggccaagaggcatcgttcgcctcttcgcttcccaccaaacggttccctcatggtagtaaatccgagcgtgtaacgtttggcgtgtacgttcgagagccttggaagcacacgcacaaagagtgcttcggcaattcggaaacaatactctttcaactagaacccattcacgatgttttccctgcctctacaatcaatacagactacgtcaccttcacgaaaccacctggcaaccggccctgtctagcatttgggtgcccacaccccaaaccgacgcagtcgcatcgcaaggagggcatacatgctttaggagccgtgtctctgttgtttgatgaatctttcgagttcggagtcttcaatcatgactacaagtcgagagggggcgctttccacactagcatcgtgaggaaatatgattttcaggatcggtttcgaatcgagaacatggaagtctggggatgtggtggtgacgaggaggccaaggcgcaagcagagagatgggcttgggaagaacgtgaagcggaagcccgtcgcaggatcaaccttgggacgggtgacattgaggcggacagggcattgctggagatggccgggctagtaggagggaatcgcagcggcggttcaatgggttga

SEQ ID NO: 21 - DNA, 46 bp, Trichoderma reesei
gcgcacgtgtgccggtcgcgcggctacaagctggtcatctacatgc

SEQ ID NO: 22 - protein, 1162 aa, Trichoderma reesei
MTQEPLKTPAQLPANNHLDSLQLPQHGSLHRDASGPGRGLPGFSLGSQRLRLDPAEHQGREAGVNAERLDEKLQQLSLDGAHDGRPSVPGQRVYEYEKASTPQASKQGLGFQVIKRSEPHADGLSLEDFPNEILTHIFSHLPPDCHSSVALVSKRFWALVTTHHAWRMAFMRFFPGHTILENNGKAAAATHSMTGPSSDVVRFETRYFPRLTPLATWRSEYLLRTRHLRSLARGKPGPPAGGSVSGRVGRGGKKSSAVLTYNSKLPWQVTNLHAVFLNGKKPPRVMQGAGDLGAATISDPTTGKIEKWGIEDLYTTPQLDEVAPNLVPYGLGDGPAGVPNVMDVSYTYGMISGEGFPGGRPYFRGVNEMRGRYVGAEISAVDTHPDIPKIPEMSDAICSVWIAKSSTVTATTQSMCGMLTGSALGIITAYSLGWDTTGPRYANGDVTARWVVSPGVPIISLKVDDGFNQKRKSSSRVWAVALNALGEVYYLCDVPVGKPGRTTGEDMTRNAWYAGRTAYWHLLEATRRVAREDEADKNATRGGYSPRSPSLDMHLSKEQIVAEAREIEKFMRYRPSHFRKVCEGWDMQRKLEVDFASDDGKGAGESIFVIDCGLAENRPVSIQRYSRSLIPVQGTPSESSTPLAPVPTSLFGSIGGFANRISPVSESQAPLSPPPTPKSPPVPSTVLHDWFKQGFEFKGHGHDTILSVALDNSLTSLYTLGEDPLHTANEASSTTSPWAEHGAREIPGRRTRFIIAGTNSGAVLVWNARDDDRTRDIQPLRILQTESPEVSAVAASGLYLVHGGSDGLVQAWDPLASTTDPIRTINARSNGRVPRHMLVMNPALQEETYSAAKAIYLDPDSTTLQGVVSFGAFLRYWSYGSNGHATGRKRRVRHADMDARLASRRQGHAVSGYIASEEAEMRREDEQQAREHNRRLKRFGALGDLTEEEALLYAQMVSQEAYHVEEQRRASDSAADASLDTASSFSENTVETLTPDPSVADPVASETSGMAEDDEYEQQIQQAIRLSLLEGVNNGVEQSPVDSSRGNSSVDFDQPVNVKYKPKGGKKGKQSGASSGGSPSASHTPVGGGASSSRLSTTEDEDLAIALSLSMQDQGGGYSPPGMSSSTMMGRSAYLEAAAGVDEEDEFPSLPGEGKGKGVQRW

SEQ ID NO: 23 - DNA, 3545 bp, Trichoderma reesei
atgacccaagagcctctcaagactccagcacagctcccggccaacaatcatctcgattccctccaactcccccagcatggctcccttcatcgcgacgccagtggccctggtcgaggccttcccggcttcagcctcggctcgcagcgccttcgcctcgacccggctgagcatcagggccgggaagccggagtgaacgctgaacggctggacgagaagctgcaacaattgagcctcgatggagcccacgacggtcgcccttctgtgcccggacagcgagtctacgagtatgagaaggcctcgactccccaagcctctaagcagggccttggattccaggtcatcaaacgctcagagccccacgccgatggactcagtctcgaggactttcccaatggttggttctgcctcttccttgcggcgagcgacccattgctgaccaatgactgccagagatcctcacccacatcttttctcatctgccccccgactgccactcgtcggtggcgctggtctcgaagcgattctgggcgctggtcaccacccatcatgcgtggcgcatggcctttatgcgcttcttcccgggacacaccatcctggagaacaacggcaaggctgccgccgccactcattccatgactgggccatcctccgacgttgtgcgtttcgagacgcgatacttccctcgactcacgccgctggccacttggaggagcgagtatcttctacggactcgccacctaagaagcttggctcgcggcaaaccagggcctcccgccggtgggagtgtgtctggccgcgtcggtcgcggtgggaagaagtcaagcgccgtcttgacctacaactcgaaactgccttggcaggtgacgaatctccacgccgtcttcctgaatggaaagaaaccgccccgcgttatgcaaggcgccggagatctcggtgcggcaaccattagcgaccccacgactggaaagattgagaaatggggcattgaggatctatacaccactcctcagctggacgaggttgcccccaacctggtgccttacggcttgggagacggcccagccggcgtccccaatgtcatggatgtgagctacacgtacggcatgatatctggggagggatttccaggtggccggccctactttcgaggggtcaatgaaatgcgtggtcgctacgttggcgcagagatcagtgccgtcgacactcatccggatattcccaagattcccgaaatgtccgatgcgatctgcagcgtctggattgccaagtcatcgaccgtcacggcgacgactcaatccatgtgcggcatgctcacaggctctgctctcggtattatcacggcctattcacttggctgggataccacgggcccgagatatgccaatggcgacgtcaccgcacgatgggtcgttagccctggtgttcccatcatatccctcaaggtcgatgatggcttcaaccagaagcgcaagtcgtcctccagggtatgggcggtggctttgaacgccctcggtgaggtttactacctgtgcgatgtacccgttggcaaacccgggcgaactactggcgaggacatgacgaggaatgcctggtatgctggccgtacagcctactggcaccttctcgaggcgactcgcagagttgctcgtgaagacgaggctgacaaaaacgcaactcgtggaggctattcgcctcgatcgccttctctcgacatgcatctcagcaaggaacagattgtggcagaggctcgggagattgaaaagtttatgcgctatcggccctcccactttcgaaaggtctgcgagggctgggacatgcagcggaagctggaggtcgactttgcgagtgatgatgggaaaggtgccggcgagagcatctttgtcatcgattgcggcctggcagagaaccgccctgttagcatccagcgctattcgcgttcattgatacccgttcagggcactccgagcgaatcctcgacaccacttgcgccggtcccgacatcgctgtttggcagcatcggaggctttgccaaccgcatttctcctgtttctgaatcccaagcgccattgtctccgccacctactcctaaatcgccccctgtgccttcgacggttcttcatgactggttcaaacaagggttcgagttcaagggccacggtcacgataccatattgtcagttgctttggacaattcgctcacctcgctctacacacttggggaggaccctctacacaccgcgaacgaagcgtcctccacgacgagcccctgggctgaacacggggcgagggagattcctgggcgccgaacccgcttcatcatcgctggaaccaactccggcgcggtgcttgtctggaatgcgcgtgatgacgatcgaactcgtgatatacaaccactgcgcatccttcagaccgagtcaccagaggtttctgctgtggcagcctctgggttgtatctcgttcacggcggcagcgacggccttgtccaagcctgggatcccttggcatctacaacggatcccatcagaacgattaacgctcggtcaaatggccgggtcccccgtcacatgctggtaatgaatcccgcattgcaggaggagacatactcggcagcaaaggccatatatcttgaccctgattctacgacgcttcaaggtgtagtctcttttggcgcattcctgcgatattggtcgtatgggtccaatggtcatgccacaggtcgcaagcggcgcgtccgacacgccgatatggacgctcggcttgcgagtcgccggcaaggccatgcagtgtcaggctacattgcctctgaggaagccgagatgcgacgggaggatgagcagcaggctcgcgagcacaaccgtcgtctcaagagatttggcgctctaggcgacttgaccgaggaagaagcgcttctctacgcccagatggtctcccaagaggcgtaccacgtagaggagcagcgacgggccagcgattcggcagccgacgccagcctggacaccgcctcttcctttagcgagaataccgtcgagactctgacacctgatccgagcgtcgccgatccggtcgcttcggaaacgagcggcatggccgaggatgacgagtacgagcagcagattcagcaggctatacgtctgtctctgctagaaggcgtcaacaacggcgtggaacagtcacctgtggattcctcacggggcaacagctctgttgatttcgaccaaccggtcaatgtcaagtacaagcccaagggcgggaagaaggggaagcaatcaggggcttcttctggtggctcaccgtctgcgagccacacgcccgttggtggtggtgcttcttcttcgcggctgagcacgactgaagatgaggatttagcgattgctctgagtctgagcatgcaggaccagggagggggatactcgccgccgggaatgtcgtcgtcgacgatgatggggaggagtgcttacttggaggctgctgctggtgtcgatgaggaggatgagtttccgtctttgcctggtgaagggaaggggaagggggtgcagagatggtga

SEQ ID NO: 24 - protein, 1738 aa, Trichoderma reesei
MLSPLRSRLVKATFLGTCRQVRLASSARYPQAVSPLAFDLHSPSHPSKDEKTAPIIFLHGLFGSKKNNRAISKALARDLKTHVYTVDLRNHGESPHDPRHDYVAMTEDLLAFIDQHGLKEPTLIGHSMGAKTAMSAALRSPETVAKVVAVDNAPVDVTLSRTFASYVRGMKKIEEAKVTRQSEADAILRDYEESLPIRQFLLGNLYRSPEDGIQRFRVPLDILGRSLDHLGDFPYKNPGEARYTKPALFVRGTQSKYVPDDVLPIIGQFFPRFQLVDIDAGHWLISEQPEAFRQEFYYLLHYTKLYPGRDTVRRTNPDPVPREAALDWPTGGARARLCPRHRHRPRPRPPNPRPPSNPPLPSVVGMLPLVQTHPRVVPVPLDFESFGAPAARPEADRRAVASPYHRGVSHSYGGPVSAAPAGQGSRDHDYHLRRKTPRGTIDAGYDGSPTQLSPGPPPLKQLILPTPSGIYPYVPPNNLPFHAKPRLSNTDPISLGQAPAVSPWSLSYGGQNFVLSSGSPMVPQPGPSWQPYGYPMFSQVPGMHQPLMRAAEYNVRAFCPPPASAPDAATFNQFGWQLGASQQNLGYFDQSQYPEPRSMLLPASFSHHTSDVQLPYRPVPDFKAGLEVTSFPSQPLVQGSYLGQSSLVEDVPAGEQYAVQASFTEKVYSKAQDHYVELLAYLQTARRLDQMSTGGNSSSSHVEAITNLAATLYCLNRQEEAEQHWLRAIKLRPDHLEATEQLVGLLYKKRSREAIDIICFVQQALSLKRGSPNRTSYPASSDDCLPSQQPSKFAAPFSYHYETASTTSGHSSRRSDFGSSGYALPNSENGRILALVHAKGTILYGLKDIERASEAFEEAVLISVGERIRSVQDLVNRIHAVLAPAGSHSTGSDRRPVSRRPLLLPPEKARQTAHLVFAADGGGSELPGLAFVPEGAARRVAVQTTSNALLSLAKIFQDAMSGGSTVPSLLRQPTGIGDILALYYLSLSLQESPSTANNIGILLAGVQQTAPSNVTTPGTVPPRPSLPGVVPGSGLALALAYYNYGLRLDPRHVHLHTNLGSLLKDVGQLDLAIQMYERAVSCDRTFDIALTNLANAVKDKGRIKDAIAYYRRAVDSNPDFAEAVCGLLTALNSVCDWRGRGGALLESGKYDRWHVDDEGTLVDVRTAMHGSGLTQRVIGIISQQLDDASQWGRGILQQPTIHGLVEQLQEFCHNPKFDLENAIRAWAHKSWEGSRLVRLVERATRVILWKWYHDRHVAQRKATPSQYYRPQLPSSLTIPSAPTVLPFHTFTYPLSAKDIRSISQRNAMRISCSTLRAPWLPPTVYPPPPPPNPYLNVGYVSSDFNNHPLAHLMQSVFGFHNPQRARAFCYATTPSDKSIHRQQIEREAPVFRDVSSWPAEKLIEQIVRDEIHILVNLNGYTRGARNEIFAARPAPIQMSFMGFAGTLGAEWCDYLLADTTAVPPSTLRPWRNNTTIEDVFQDMTEGDERQWMYSENIIFCRDTFFCCDHAQSCDDNERDMTWEDEERRRWKMRKELFPTIADDAIILANFNQLYKARETNLRQTAEAWAGAEVASRLVFTDVAPKNQHINRARVCDLFLDTAECNAHTTAADVLWSSTPLLTLPRYSYKMCSRMAASILRGALPKSAEGQQAALELIADGETEYEDQAAELAGGLTYVMTDEGYGRGKGRLAELRKLLWDSRWSCGLFNTRRWVNDLERAYEEAWRRWVAGKGGDIYL

SEQ ID NO: 25 - DNA, 7056 bp, Trichoderma reesei
atgctttctcccctgcgctcgcggcttgtgaaagccacattcctgggcacatgtcgccaagtgcgattggcctcttcagcgagatatccccaggccgtctcgcctctagcctttgacctccattcgccttcccacccgagtaaagatgagaagacggctcccatcatcttcctgcatggtctctttgggtccaagaagaacaacagggcaatcagcaagtaagcgatgattcatcgtgtgctgtcatggtctctagcttacaatatcgtagagccctggcccgagacttgaagactcatgtatacacggtggtaggtgaagtctgcgaggctgttcaagcaagtttcatcagcgactaaacaggaatgaaggatctgagaaaccacggagaatctccccacgatccccgccacgactatgtcgccatgaccgaggacctgttggccttcattgaccagcatggtctcaaagaacctactctgataggccattccatgtacggctcatctgagactgcgggaagccctgacagccggctgatgtatggattgcaggggcgccaagacggccatgtcggcagccctgcgctcgccagagacggtggccaaggtcgttgcggttgacaacgcacctgtcgatgtcacgctgagcaggacgtttgcttcctacgtccggggcatgaagaagattgaagaggccaaagtcactcgccagtccgaggccgacgccatcctgcgagactacgaagaggtcaatgccgccgctgctccttgatgcgaatcatcgccgagacaagaactgacagtgtcttgcaaacagtcgctcccgatccgccagtttctgctcggaaacctgtatcgctcacctgaagacggcatccagaggtttcgcgtccccttggacatactcggtcgatcactcgaccacctcggagactttccctacaagaacccaggcgaggcacgctacacaaagcccgccctctttgttcgaggcacccagagcaaatacgtgccggacgacgtgctgcccatcattggccagttcttcccgcgctttcaattggtggacatcgatgccggccactggctaatatcggagcagcctgaggccttcagacaaggtaatttaaatcaaatcacaaacaccaacacacttgtccggatctgcgttaatgacacacacccacaacagccgtcgtgagcttcttgcaagatcctgaaacagcgcaatagagagagaaaggtgagattgttagaccattatagagcaccttactttagacgattatataatgtgtatcttgcgatttccatcacttttgaatgcttgaccccatacctagaattctactaccttctacactataccaagctgtatccaggtaggttttcatgtacccgtactcgtaccttgctagtgttggtcctgcattcggtcacagtgtcaggctgccgttcatcctcataccagattgcgatgttgcagcatctctcctaattgccaaacaggccctttgcccccttttggtgagggaaaccgcatatctcgattccttggaagcactcgagcaaacaaatgccttgtaccccaaaggtattccttggctgacacatccccccgcactgtgtacatgcagcttgcatgcccgagtaacgcaggcgttagccagtgccacccactctacatcataccccccctgcttgattcacccagcctcgaggctccctagctcagcccgtttgtcactactactgcacgcttcccttgccttgctcgaaactccatgctgcgtctgcgtctgcgtttgctcgtttgcaaggtagagatacagtacggcgcacaaacccggacccggtacctcgagaagcggcattagactggccgacgggagggtaagtcagtgtcaagtcacgtcctctacctgtgccgctgttgcagctgctggccccgtgcacatcagcgcagcgcctcagcccccgatgcctgccggcgtcctcagccatgatgtgaacggcatcgacattgctgctcgtccgcaagtggttctgcttctcgctccttgactcgacctagagcccgcgctcgcctttgtcctcgccatcgccatcgccctcgccctcgccctccaaaccctcgcccgccatcgaacccgccgctaccatccgtcgtgggcatgttgcccctagtccagacgcacccgcgcgtggtgcctgttcccctcgacttcgagtcttttggagcgccggctgccaggcccgaagctgacagacgagccgtcgcgtcgccctaccatcgcggcgtctctcactcatacggcggccccgtcagcgctgctcccgccggccaaggctcgcgagaccacgactaccatctgcgccgcaagacgcctcggggcaccatcgacgccggctacgatgggtcgccgacgcagctgtcgcccggcccgccaccgctcaagcagctgatccttccgacgccctctggcatctatccctacgttcctccaaacaacctgccctttcacgccaagcctcgtctctcgaacaccgacccgataagcctgggacaagctcctgcggtctcgccgtggtccctcagctatggggggcagaacttcgttctctccagcggatcccccatggtcccccagccgggaccatcatggcaaccctatggataccccatgttcagccaggttccaggcatgcatcagccgcttatgagggcggcagagtacaatgtgcgtgccttttgtccgcccccggcctccgcccctgatgccgcgacattcaatcagtttggctggcagctgggcgcttcgcagcagaatctcggctacttcgatcagtcccaataccctgaaccgaggtctatgctgcttcctgccagcttctcacaccatacgtcagatgtccaactcccatacaggcccgttccggatttcaaggccggcctcgaagtaacttcatttccctcacaaccattggtgcaggggagctatctgggccagagctcgcttgtggaggatgttcccgccggcgaacaatatgccgtccaagcgagttttacggaaaaggtctactcaaaggcccaggaccattatgttgaactgctggcctacctgcaaacggctaggaggcttgatcagatgagtactggcggcaattccagctcaaggtttaaagttctggtatttccaaggcccccaaagtctagaaaggagcattttggtacctttcgaggtttaaataaccgttttgagcggcctactgtgtcggccatcatgccaaccagccatcctggggcattgacatcgaatatcgaggatactctgagcaacaacacagggccatcgatgggacaagatgtccatgtggaagctcatcggcgtcgtacctcggcaacacattttcgtacctcatccaacgtctacaccccatcctcgtcatttggcgcagttggtccggttgccatggcgaaagcgttaccaattctgaacgcaaaaagctcgtttgacatgttgaagaatctatgtgagcagagtaactggaagtggattgatggaatcttactcggtgggtgtctgttatatggcctctcgcggttcgaggatgccgtggagtggttctccagagttcttactctagattcaaggtttgacctgttccagatgaactatcaagtggttgttctctatctgactcagattatagccatgtcgaggccatcacgaatttagccgctacgctctattgcctgaaccgccaggaggaagctgagcagcattggctgagagccattaagctgcgccccgaccatctagaggccacggaacagctcgtcgggctgctgtacaagaaacggagccgggaggcgatcgacatcatttgcttcgtgcaacaagcgttgagtttgaagagaggaagtccaaaccggacgagctaccctgcctccagcgatgactgtcttcctagccaacagccatcgaaatttgcagcgccctttagctatcattacgagaccgcctctacgacgagtggccatagctccaggcgctctgatttcgggtccagcggatatgcgctccccaacagtgaaaacggccggattttggcactggttcacgccaaaggcacaatactatatggtctgaaggatatcgagagggcttcggaggcgttcgaagaagctgtgctgataagtgttggggagcgcatacgaagcgtccaagacttggttaaccggatccatgctgtcctcgcgccggcagggtcacactcgactggcagtgaccgtagaccggtttctcggcggcccctcttgctcccccctgagaaggctcgccaaactgcccatcttgtatttgctgctgatggcggaggtagcgaactgcccgggctggcgttcgttcccgaaggcgcagcaaggcgggtcgccgtgcaaacgacgagcaatgctctgctctccctagccaagattttccaggacgcaatgtctggcgggagcactgtgcccagtctgctgaggcagcccacgggtattggcgacatcctggcgttgtactatctttcgctttccctccaagagagtccctctacagcaaacaatatcggcatcctccttgcgggcgtgcagcagacggcaccaagcaatgtcacgacgccaggaacggtccctcctcggcccagtctccccggcgtcgtacctggcagcggccttgctctcgccttggcctactataactacgggctgcgtctcgatccgagacatgttcacctgcacaccaatcttggcagccttctcaaggacgttggccagctggacctcgcaatccagatgtacgagcgggcggtgtcatgcgacaggacgtttgatatcgctctgaccaacctagccaatgcagtcaaagacaagggtcgcatcaaggatgccattgcctattacaggcgagcggtggactcgaaccccgatttcgccgaagccgtctgcgggctcttgacggccctcaactccgtatgcgattggcgtggcagaggtggagcgctgctggaatcgggcaaatatgaccgatggcacgttgacgatgagggtaccctggtcgacgtgcggacggccatgcatggcagcggcctcacccagcgagtgattggcattataagtcagcagctggacgacgcatcgcaatggggccgcggcatactgcaacagccgaccatccacgggctggtggaacagctccaggaattctgccacaaccccaagtttgacctggaaaacgccatccgtgcctgggctcataaatcgtgggaaggctcccgcttggttcgcctcgtggaacgggcgacaagggtcatcttgtggaaatggtatcatgatcggcatgtcgcccaaaggaaggcgacgccctcgcagtattatcggcctcagctgccctcttctctcaccataccatctgcgccaactgtgcttcccttccacaccttcacgtacccgctatctgccaaggacatacggtccatctcccagcgcaatgcaatgcggatatcgtgctcgacgcttcgggccccttggctgccgcccacggtgtatccgccaccacctccgccaaacccttatctgaacgttggatacgtgtcgtcagacttcaacaaccacccgctagctcacctgtaagaagcatccctcttgcgtgtgacgccttggctcgtgaagcacagtatgctaacatgcttcggcaccatcacaggatgcagtcggtatttggattccacaaccctcaacgtgcccgggccttctgctatgccaccactcccagtgacaagtccatacatcggcaacagatcgaaagagaggcgcctgtgtttcgtgatgtcagttcctggccggcagagaagttaattgaacagattgtccgcgatgagattcacatactagttaatctcaatggctacaccagaggtgctcggaacgaaatattcgctgctcgcccggcccctatccagatgtcctttatgggattcgccgggacgcttggtgcggagtggtgcgactacctcctcgccgacacaacggccgtccccccgagcactttgcgcccttggaggaacaacaccaccatagaagacgtgtttcaggacatgaccgagggggatgagcgccagtggatgtactcggagaacatcatattctgccgagacacgttcttctgctgtgatcacgcgcagtcgtgcgatgataatgaacgcgacatgacgtgggaggatgaagagaggcgtcgctggaagatgcgcaaggaactatttccgacaatcgcagacgatgccatcatcctggccaactttaatcaactctacaaggcaagtaaagcttgcatgaaatggccagcttgtagtactcttcccagctaacctgatggtagattgacccgactacattccgatcttggttacgaatcctcgccaggactcccaaggccatactctggcttctacggttccctgaactaggagagaccaatttgcgacaaacggccgaggcctgggccggggcagaggtggccagtcggctcgtcttcactgacgttgcgccaaagaaccagcacatcaacagggctagagtatgcgatctgttcctcgatactgcggagtgcaacgcgcacaccactgccgccgatgtcctgtggtcgagcactcctctcctcaccttgcctcggtattcgtacaagatgtgctcccggatggcggcgtccatcctgcgaggcgcgctgcccaagtcggcagagggtcaacaggcagcactggaactgatcgcggatggcgagacggaatacgaggatcaagccgcagaactagccggggggctcacttacgtgatgacggatgagggatacggtcggggcaaggggcgtctagcagagctacgaaaactgctttgggatagcagatggagctgcggactcttcaacacacggcggtgggtaaacgatctggaaagggcttacgaggaggcatggcgacggtgggttgcgggcaagggcggtgacatatatctgtg

SEQ ID NO: 26 - DNA, 5649 bp, Artificial sequence
  DNA fragment for transformation.
cttaagacctgtatcctcgtacagcatgcaccaagctcaccggcgagtatggtatcgtaccagcagcactccctcctccctcctggacaaatctgcatccaaggtcccggtctatagggccgcggcaacaacaccagagtggaccaccaagcacgtgtggcaaggtgggctgggacgctccatatccacaagttcaggtccggagcgacgctatcgaatggtccttttggggacttcctggatcatggaccacccccccctccggccacttttgactcgagactgataccgaccaacggcctagctagatggtagttatttgccttgtatccacacagcagcacccataggcgagtgcttgcccaggctttctgtacaagcacgaattccgtccgttacagggcctcagccgcgtccggttcattgggaactgcccttgttggtctcgtttgccttttggggttgcttcggtgtatctactctatttctgccgttcacttggctggaccctgctagctcctctctgcaaccctgtccgcttctcgagactcttttcgttttgtgggcagtggtggcctagatctcaaaagccactgataccctcctcgtaacctcgtcgcccgcatcttccccatctcctcccccagattctcaacacgtcgccacttgccgctgggccgttgactcgagatctttcctcaaggtctagcaacctgtctactcttgtcttattccttcatggtccattcgatatcgagactctggctggaagaagtgtcatagaacacgcactcacctgatcacttacaagcgtcgcgcccggtttcaaccttggatcactgcactcacttgaccagcaaatactattctgggcgattcatacgttgttgcgcgtgtggaaactttgccctccatacctggcctgtggagcaacccggttggacacctgaaggacgagagtatctgtttagccgctcctttttgtcgctgggcgcttcttcttcctgaaggatcatctcgtgcttccttggagatgcaactctaacagctgccgttgctccaacaaatagtgtctatcttcctctcaatgggtggactaccaactctagaatctctgcatcgcgctcccccgccagacactccacctgagctgataagcccaagtgcaacatcattctcttctaggtcgtcgcctcggtaccgcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtccagtaatgactcatgcagaggacatttacattcgacttcctgccgacctcaatagcttcgagaagcaagccgagtcccgcgcgcgaagctttgacccatattgtggccgcgtcgtgtcggatgagccatcgtcggacgccactgtcacgggctactatcttgaaatggtgcacatctggtcggactgccaggcctccatctatagaatggccctcagacggacgcccaccgaagccgagaccacgaagctgcaggatctcatcaagagagccaagaactgggctgcttctctaccccctcgcatgacgtttagtggcacaaacatcgaaacggctgctttctcgagaagcacggggtctttcctcagcatgcattttctttaccatcataccatgatcgagttgaaccgacaccgctacggagccggccagctaccccgggatgtccagttggggcattcggccgagtgccgggagcacgcaagccgggttctcgagatgctgaacagtcttgagcgtatcttgagggtcaggtctgcattgctgagcatccctcctccagccatggctgtcgccgtcacagaagctgtcgatgttctgactgccagtggtccgatggccgttttaggcgagatcattgaccgagttcgaatcgcccagtcggcaattgacaagatgaaggacgtctgggaggcatcctcaaaggacggtcttgccatccaccggcgtttacaaaagttgaaccgcattcgtgagctaggatcgcggccgcccagccctattcagggctatcgcttgcttcccctgtcagacaacacaaaggacaaggagctcagccactggcagatctttgatccccttgaacaaacatttcccagagacatggacgtggtctatgtcggatgcgactagacgcttggagcttcggcgtctgtgtgctgcctctactggatgacttgttcctattgtcgtgtatacatgtgtatacatgcacgtctactttctatatctatccgcttttttctgtattcctcttctcttgtatttcttctctttactgtctatgttttattggcctgtttgtttttacccaaaatgagcttgcttttttttggaaacgactttaacgtgttatgcatggtttagcgtttagatcgctgcccacttgggcctgcttggaggcatcgagatcaaggcgacttgaatcggatgacatgggatgaggagtttgggcattcgatgtgacaaggaggccttggtcgcgaatctgtatgttgaatgggaggctacttactctgaatggcattttacgtttgatgccgcatatctgtatatcgcctactagt

SEQ ID NO: 27 - DNA, 37 bp, Artificial sequence
  Oligo DNA for PCR.
atgcatcttaagacctgtatcctcgtacagcatgcac

SEQ ID NO: 28 - DNA, 33 bp, Artificial sequence
  Oligo DNA for PCR.
atggtaccgaggcgacgacctagaagagaatga

SEQ ID NO: 29 - DNA, 33 bp, Artificial sequence
  Oligo DNA for PCR.
atacgcgtccagtaatgactcatgcagaggaca

SEQ ID NO: 30 - DNA, 33 bp, Artificial sequence
  Oligo DNA for PCR.
atactagtaggcgatatacagatatgcggcatc

SEQ ID NO: 31 - DNA, 5897 bp, Artificial sequence
  DNA fragment for transformation.
cttaagtacctagggtagcttggtactgcagctgaacacatacggaccgtttgttcccacaaaaggctagcactcattattacggagggagcagcatttccctcagttttgatcgtatggagcagtcaaacaaaagactggtggagcgacccattggactggatacgtgtgtgatgctactactactcgtactactagtatccggaatcgggaccgcgagagccccccaaaggaccaaaagaaactcagggacctgtcagcagccaggcgcgcaagctaaggcggcaagtgcttagaaaccgggtcctcaagtcgagaggattcgatatctgccgcgggagggatgaagattttggcttttgtgcgctgtcaaagttcgagatgctgggtggtttgttgtgggtgtggcttgcgattgatacagtaagccaggtaatacctcacctatctaggtaaaaacatgcattcagcggtgccctaatatgtactgtagatagataatccggtaatttgtaggtaaggaacttgcggagttcgcctcagcctgagtgatctttgtgttccctgttttgctgagcaagtcgagtcctcgtgcaatgcgatggcatgccctgtggcaaaataccacaatgaaccctcgccatacaccaaaagcaggcgaccaattgggaacgggctacagggaaaagcactcactacctatggcctcgcaagggggctcgtgtgattgaaaagagtacctactcgtacacggaaccttggccggacttgacgaggtcccacgcgggcatcgcggaacacctcgagtaaatactacggaagaatccgtcctgacgtaggtactggtacgggaatgtatttcgatgaggagcagtcggacgccgcaagtacaggcacggctttacttttaggttcaaggtttgatatgccttagacactcttgatctggcggcactcgggcgagaggcgggagaggcagagtaaggcaaggcaaggcaaggcaaggcaagacattatagttgtagtagtactactagtagtaagtactaggtagtaatcgacagagagcagcttaccttaccccccctcttatgtacccgaacaacaagaaaagggctgggctaggtaaggtattagaggtacctactgcacattcaagatacatgtagcccgacagagaccacctgaaggataccttgccctagtcgagcacagcggccttgatctaccccgctagtggcttgtcaggcgtctcccttcaggcctcttcagtggctccagtggctccagtggctccagcgcccctccccgtgtttttcgccttaccccagcctattgcctcgatcgctcgcagctggagccagtcaaaggcgggcggaagccggtacaagtacagtatgtgcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtgtcgacactagtgcatgcctcgagatttaaatggttcagtggattatcgttattgggcatcactgggttttggcgtgcggaatcttacgggacgagagcttttttctctcgacatccccttatcgtgtatagcatttgctcgtttatcgaggcggattcgttctctcgttttatgaaaaagtcggccttcctctgatgtataacctggagatgaaaatatttgattcgaataccacctgcagtatgtatgatgttctattatagtaattgcttcagttgccggcttctacaaggacatagatgcttggggcatctgctttgtgtcgagtaagactggcagcagagaattcaaacgaggtgactatgtatgtaattttgattaaaagaagcggggtgtgctgtctatatgtctttgatagatggctttggagccatcagtttactttgatgtcgtaaagtaattccaggatgattccaggtgattgacaataggtatgacgggcagaacgtgttcttgttaatagggtaaggccaaataacaccaaagagggtcgacgcatttacccacagctagctacgtatcgtacgtacttggaatcatccctactgctcaagactgttcgtttgattcgaggaggggggctgaagagcatagaggccaggagcacgatattcaaagaagaaaagaaataaataataataataaaaaaaattaaaaagaaagaaagaaaaagaaaaattaggaggtcagcggaaatagatggatggtgattggacgatgggcggctttgcatcatttggggggaggattgtgctttttctctactttatttctttcgcacctgtactttttaatgtggttggctgagtcacttttattctgtaggtcctagtattttatatgttggagattctgcatctccgcattcgcttttacctggcgacttgacgtgagttgattgaggtaccttgtggatgggattgtctgtcaatcacggtgatcgcaacaagtgagattcatcaaggttccagtattattccgttcagccccctttttccaaggtaaggtacatcatacgcgtacgagtacgaagagccaggccaggccacaccgattctttagacgaacagagagtatcaactgacttgactccctattacataacatgcggcaacaaaagccaaagaatcaagaacagagcaaatcaagttgtttacagtaagtatccaggttcttgctcaacagctgtacatgctcgtatgattgctctctcagcccgagataaaagaagagccgttattcttaatccggtagcattgtacaattccgccctgtaagaagaagtacggtacactgagataggtagttgtactgagcttagcctgaggcgcgcc

SEQ ID NO: 32 - DNA, 37 bp, Artificial sequence
  Oligo DNA for PCR.
atgcatcttaagtacctagggtagcttggtactgcag

SEQ ID NO: 33 - DNA, 35 bp, Artificial sequence
  Oligo DNA for PCR.
atgcggccgcacatactgtacttgtaccggcttcc

SEQ ID NO: 34 - DNA, 39 bp, Artificial sequence
  Oligo DNA for PCR.
atgcatatttaaatggttcagtggattatcgttattggg

SEQ ID NO: 35 - DNA, 34 bp, Artificial sequence
  Oligo DNA for PCR.
aggcgcgcctcaggctaagctcagtacaactacc

SEQ ID NO: 36 - DNA, 6169 bp, Artificial sequence
  DNA fragment for transformation.
cttaagaatctcctcttcacccacgactgtcgccactctgattttgctcaaggcatcgcaagcgattgattcttctcgtcctcttacctcgcagatattcatcaccaacctacccggtcccgcctccctcaatgccgccgccggagagcagggcgttgcgctctccccctgggaggtgctccattcccaggtccaccacgccctggtgccctacttcgacgccaacagcaagagtcagctgctggccaatgggtccaggggaagggacgtcgatgccaagacgggcattcccgtcaccaagaagcgcctcaacgacctggagctgagcctgctccacctgcagcagaacgtcgacattcccgagatctcgctgacgttccactcgctcattcagaacgtcctcgacgatgccgaggtcaccggtacgaagccctccattgaggccatccccaagaacctcctccaggacagcagcttcctcaacaagctgcaggccaacgtcaacacctggatcaagtccatccagggcatcaccaagctgaccaaggagacctcgtccaacgccgtccaggagttcagcaccgccagccaagaggtcaacttttggctctccatggagtctgcgctcgagggcatcgaggaccagcttaggagcgagggcgtcctcctcaccctcgatattctcaagcacgccaagcgtttccaggccaccgtcagcttcgtcgccgacacgggcctcaaggaggccaaggaaaaggcgcaaaagtataaccagctcatgcgcgatttccccctcgacgagctgctgtccgctccgtcgctgctcaaggtcgaggaagccattactcagatctttgcccacctcatgaagaagctgagggtgtgtccctaccctatccggcgagcactgagcttggcccaggccatctcggcggatctcaacgacgtgctgctccgcttgcttccgggcactgagctggtcaacatgggctacccagagttccagaacgtcatgcggacctgcgacagcatctttgccgcctgggaagacaacatcaaggagtttacgcacctggctcgtatgctcatcatccgcagaaacgagaagcgcatccccatcaacgtcgagaagaaccactctgagctggagtcgcgcatcaagtacgtcagcgcattccgagacaaccacgaacagctacagcggaccattgtcaacgtcctggggccaaaggccattcttcccggcgttaccgacgtcaccgcctcctcctcctccactgccactgccggtgctgtcattgaagaaatgggcgacgtagatgccgtcgaggaggtcaagcgggcctgggaggcgctgcagaacgtcgacctgctcgatgtgacggaccagggcaaggagcggtgggcgcaggccgaaaacctgtacaatgagcgctcgacgcgagtcgaaaactccatcattgcgagactgcgagaccgcctcgccacggccaagacggccaacgaaatgtttcgcgtcttttccaagttcaatgccctgttgcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtgaaatgctgaacaagatccaaaaggccctgggtgaatacctggagaaggagcgcgtttctttccctcgattctactttgttggtgacgaagacttgctggagatgattggcaacagcaacgataccttgcgcatcgccaaacacttcaagaagatgtttgcgggcttggctggcctcattatggatgacgagggagtcatctctggctttacttccaaggagggcgaggccgtgacgctgaacaaggagatttcgctggccaagacgcccagaatcaacgactggcttgcgctgctggagaacggcatgaagcagacgcttgcggagcttttggccaaggccgtgaacgaatacactccgattttcgagtctgacaatattgaccgggaagcgctcgttgcctttatggatgctttccccagccagattgtcgttctcgccacacaggccgcctggacgaccgccgtggattcgtcacttgcggctggcggacagacgctcaaggctctctttgatcgggaggtgcaggtgcttcgcgtccttgcggagactgtactgggagaccttgaggttattcagcgcaagaagtgcgagcagctgatcaccgagtgtgtccaccagcgcgacgtgattgagaagctggtcgacgtcaaggctgattcggccgaccactacctctggcagcttcagatgcgctacgtctacacccctgagggtaacttcctcaatcgcctgtacatcaagatggccaatgccaagctcaactacggcttcgagtacctcggcgtgcccgatcgtctcgttcggacgccccttaccgaccgctgtttcctcacgctcacccaagccctctgccagcgactcggaggctcgccctacggccccgccggcacgggcaagacggagtctgtcaaggccctgggtgtccagctcggacggtttacgctcgtcttctgctgcgacgacactttcgacttccaggccatgggacgtatcttcttgggtatctgccaggtgggtgcgtggggctgcttcgacgagtttaaccgtctcgaagagaggatcctgtctgccgtttcgcagcagattcagaacatccagctcggcctgaagcagggcgccgagaacgacaagtcgcagattgagctggttggccggcagctgcgcgtcaacgagaacacgggcatcttcatcaccatgaaccccggctatgcgggccgttccaacctgcccgacaatctgaagaagctgttccgcagcgtggccatgtccaagcccgacaaggagctcattgccgaagtcatgctgtactcgcagggcttcagccaggccaagcgactctccaagcagacggtgcccttctttgacaagtgttccaaggagctgtccaagcaggcccattacgactttggtctccgtgcgctcaagagtgtcctggtgagctctggcggtctcaagcgctctcgcttggtagacggcggcgaccttggcgccgaggagattgttgagcccgagattctactagt

SEQ ID NO: 37 - DNA, 37 bp, Artificial sequence
  Oligo DNA for PCR.
atgcatcttaagaatctcctcttcacccacgactgtc

SEQ ID NO: 38 - DNA, 36 bp, Artificial sequence
  Oligo DNA for PCR.
atgcggccgcaacagggcattgaacttggaaaagac

SEQ ID NO: 39 - DNA, 33 bp, Artificial sequence
  Oligo DNA for PCR.
atacgcgtgaaatgctgaacaagatccaaaagg

SEQ ID NO: 40 - DNA, 33 bp, Artificial sequence
  Oligo DNA for PCR.
atactagtagaatctcgggctcaacaatctcct

SEQ ID NO: 41 - DNA, 5931 bp, Artificial sequence
  DNA fragment for transformation.
cttaagaagaagtgggaagaagacgtctggtggttgagactggccgggaatggcggctcatctcgacttcggcgcctctgcctcctccgcgacgccgccgcagcaacagcagcagcagcagtcgcacccgcatccgcacccgcacgccatggccgaccacaacgtctcgtcgccgcaccacccgagcaacgtcaccgtctaccagacgacggggcccgatgcccactatggccccgtcatggcccccggcgatgcgggcacgccgctgctcaacccgcgatcctgcgtcacctgccgccggcgcaaggtgcggtgcgacaagcagatgccctgctccaactgccgccgcgccctgattccctgcgtctttcccgcgcccggccgcgcgcctcgccagcaccggcccaaggaccccaacgcccctcccaaggccacgagccagcgcgaggtcgagctggtcaagcggctcaagaagctcgagggcatcgtcgaggagctgagcggccagatcgagatcgagacgggcggcagggtctcgtccgccggcgcctcgccttccaatgacggatcgccgtcgactcagcgggccaagtctaccagcctaggcgcaatgacctcgggccttgcggatcatggcgacggccacagcgccggagatgggagcgaatcgccaatgatgagggagagccggaagcagctgggccgattggtcctgaacgataacaagggcacgtctcgctatgtgagcagtgggttctggtcgaggctgaacgacgaggtacggtgcccctcctgctgtcccacccccttgctttacccccccgcgtgataccatttggtgtgtgctgacatttatgcctcctcatcaatcagttggatgccatccgagaggaaacacaaaaacttaccgacgaagacgccgaggactctgactttgaagaatcgccgcccattgactcccccttcaccgccaccagcagctccgtctatgaccaccatggcttcatcctgggatatcgatcaatcgatgtcaacatacagaaatgccatcctctgccgtcccacggcacattcctctggtcggtctacctagaaaacgtcgatccgttactcaagattctccatgtcccgaccatggaagggatactgcgggacgcccggcggaatcctgaaaagctgtccccaggtaacgaatgcctggtgtttgcggtatactttgcggccgttgttacgctcgacgatgctgatgtaggtgctcccatgcctggacattgagtttcccaggactctgctaacatcatcaggtccgaaccaactttagtattcgcaaagaggaatgccttgctcaatttcgattcgccgtggaacagtcgctcgccagggccaactttctaaacacgtccgacatcacagtcgtccaggcatttgttctattcctcctggtgcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtttctacaagaggatcgaagacaagttcctctcgcacctggacgaggacgttgacgcgctgtactgggttgcagccatgatatcacgcatcatcatggccaagatgtgcctcatcatctaccagccgatgctctttccaggaacgggcagcgagcctacggcggaaatccgagaccggatttacattgcctcgattgagattgtcgagtacaaccacaaactgaacctggaccccagaggcaagcagtatcggtggctgtttcggacctacaccaattggcccgcgattgcttatatcctggtggagacgtgccgccgtccttggtcggcccttgtccagcgcggctgggaggccgtcgtcagatacgacaaggacctcacagaaaacatgaagacggccgatcacgcctccgtgttcctcccgctgcgcaaactgttcaccagagccaagagatatcaaacaatggaggtggcgcgtctcagggccaaccctgaagaggccaggcgcctggatctcgcggagaggatcaaggcatcccaggcgcgattcgatccgatcccgggcgccgaggcgaggatgcagcaggtcagagaccgatggagaacgatggtcaatctggaaggggccatcccagcccctccaatagaacaagcacccatggtgcctcctgtatctgaagaggctcatccaccatctcagcagacacataccgaccccggacagctctaccaacagtcgcagatccagcctcaggtcagtggcgaaagttcagtccccatggacctctccaatgtcaccatggagtacatgaacgccattatggcgcaccccagcgtccccatggccgagctctggtcggtgtcgttgggagaagacccggccatgagtatggacggagggccgcagggccaggacaacatgctcggccagcagccgatgatggcgacaaacccgcagcaagccaagggcgagcatcatgttccgccctatctctggcctgagtcctttgcgacgcccagtcagaagtttgacctggaagatgcggacatgctgggggctgactttaactggcatgactggagccagagcgtccgcggcctgatggatggggggcagccgaagccggggtggtgaaggcagccatgtgtgtgtccgatttgacatgtatcacgagcttggggtgttcttgacgttgtattaccatgcatccgtaccgagctaattgggtgagggagaagcaatcaagctcctggtgtgtttatatataggtatctatggcgtagggggggaggttgggggactcaatacctcacgatacccttgggcgaatcaggcgttgttcattatgggaattagacatgtgtgtatgagtgtcacctgactagt

SEQ ID NO: 42 - DNA, 37 bp, Artificial sequence
  Oligo DNA for PCR.
atgcatcttaagaagaagtgggaagaagacgtctggt

SEQ ID NO: 43 - DNA, 35 bp, Artificial sequence
  Oligo DNA for PCR.
atgcggccgcaccaggaggaatagaacaaatgcct

SEQ ID NO: 44 - DNA, 33 bp, Artificial sequence
  Oligo DNA for PCR.
atacgcgtttctacaagaggatcgaagacaagt

SEQ ID NO: 45 - DNA, 33 bp, Artificial sequence
  Oligo DNA for PCR.
atactagtcaggtgacactcatacacacatgtc

SEQ ID NO: 46 - DNA, 5553 bp, Artificial sequence
  DNA fragment for transformation.
cttaagtacatcttgacaaagagtaagagccattccaccgaaagagcaagcaaacaaagattgattgcaaacagacacgtaattgtaactcctgcgtgcgtaccaaagttgctattaccgcaaagagccttttgttcaaatgtcaagaagtctcgtccaattcggctgaaactacgagaacaatcaaacaagacgcttgagaggacatataatacgattcaacatgttaaataaaactaaatcatagtagaagcgtttatgttgtatctagactattgaaggcctagaaggatatacatcgcttcatgttgttcgttcgaccttttcctcgtagttggctctcatcagccgtcacgtagatcagaataagacgtgatgcatattatcgtcacagtaattgccttttctactcccgtttgtgattcggcgacgaaacgggattacaataccaagatgttgtgataagcacaggcggaatacacttgaagaagatcatgacaagaattctactatttattcgcgccactcatcaccaaaacactttgtccatcccatgctaacccttcttgtttgttactaccgtcaagatcttgcttctctctgccaagtttgcaacgatatgcaagcaaaaccaagagtgaatctgcctcccacgcgtcccctccaacatctattccaatctcagccccggcgcattctacctagctgagtacctaggcagaacgccctctcttctcaactctatccatgaccataaaaagatacccaagatggaactaagcccctcacaaactaaaggggcagccaagacaatgttgagcgccgaaacaaccatacaagcaatggtataagaggcattccgcccttcctcattagcctcaaacctcctgagccaccgaatgtatggaaagaggagtacgatatgaggtatcacgttaaggacgcgggtataacgcagccgactaggtttctcagagcttgacgggtcctggaatccaggactcgtagactcatttctcatccaccatgccaagactatggcacagcaagcattgcacaccatcgccagcgcactccccaagtgatatgctgcgtcaaaggcgtagaagactatggccatgaggcagtatcttcgatcgattttccggtgctgctggtcaagttcgtggtcgcggatgtctcgcagccagatggagagtatgatgagcccggtcgcgaaggaagtgtggaacagccatcgaaggaatggtatgagcagagcgagccctaacgcggcatcttcagcctcgatggccatgatgcagggtgtaggagtgtagggagagatgaatatgcgactttgctggatatgctgggggcgaagagaaaggacggtggtagttggtgataacgaaacagcccgttaacggatttggagagaatggatagcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtgtcgacccttcacccacttcttcttcaaggagcccatcttcaaaacagccaccacgttgcctctgccgtggcaaatcttcaaacacgtcctcatcctcctctccgcgcgcgaatccctcctctacgccatccaccgcttcgtcctgcacaccaagtcctcaaacgcaaccttgcgcgccctctccaaacgccatcaaaagtacgcccacgcaaaggccggagcacccttcagcctccgcctcctcgcggaccatccgcttcccttgctatgctacaagttcatcccgctcttcctccccgcggcccttctccggcctcacatcctcacttatttcctcgtcttcctgctctgcaccggcgaggaaacgctcgcaatgtcgggttacaccattgtccccggcatcatcatggggggcatcgtgcagcgcacggctatccactacgcgggcaaggggacgtccaactatggctcgtgggggatcctggactggatcaacgggacgagtcgcggtagggatgtgctggaggacgtcaagaaggaggcggataagcatcagctgcaggaacggtctgcgaagaaggtaaaccagggggctggggccgtccaggagggttttgacaacttcagggagggggtgaggacaaggagaggggggaggaagaaggcttcgcagtgattacggggttgtttgccttttctctctctctctctctctctctctcttttttcttttttttgttcttatcgttccgtttttggttatgagcaaccttgataccactataagtctgtttactgaaaagtcacattgttacccgctgtcatcatttcggctttgctaaatcagcgatcaagaagttgctgtatgtgccctatacgtgagctgtgaatatctgtgaaatcagcctcccaaccatgcctgggttgtcctctttgacggacacctccctctagtaagtctataacacttactcccccagcataaaacgccgtatgagccgtccgtatgtgtatatgagcatgc

SEQ ID NO: 47 - DNA, 37 bp, Artificial sequence
  Oligo DNA for PCR.
atgcatcttaagtacatcttgacaaagagtaagagcc

SEQ ID NO: 48 - DNA, 35 bp, Artificial sequence
  Oligo DNA for PCR.
atgcggccgctatccattctctccaaatccgttaa

SEQ ID NO: 49 - DNA, 35 bp, Artificial sequence
  Oligo DNA for PCR.
atcggtcgacccttcacccacttcttcttcaagga

SEQ ID NO: 50 - DNA, 33 bp, Artificial sequence
  Oligo DNA for PCR.
atgcatgctcatatacacatacggacggctcat

(19) United States
(12) Patent Application Publication
(10) Pub. No.: US 2021/0388410 A1
(43) Pub. Date: Dec. 16, 2021

(54) MUTANT STRAIN OF TRICHODERMA REESEI, AND PROTEIN MANUFACTURING METHOD

(71) Applicant: Toray Industries, Inc., Tokyo (JP)

(72) Inventors: Yusuke KAGAWA, Kamakura-shi, Kanagawa (JP); Shingo HIRAMATSU, Kamakura-shi, Kanagawa (JP); Katsushige YAMADA, Kamakura-shi, Kanagawa (JP)

(21) Appl. No.: 17/283,299

(22) PCT Filed: Oct. 10, 2019
(86) PCT No.: PCT/JP2019/039935

§ 371 Date: Apr. 7, 2021

(30) Foreign Application Priority Data
Oct. 11, 2018 (JP) 2018-192641

(51) Int. Cl. C12P 21/02; C12N 15/80

(52) U.S. Cl. CPC C12P 21/02; C12R 2001/885; C12N 15/80

(57) ABSTRACT

A mutant strain of Trichoderma reesei has a mutation that eliminates or reduces a function of a polypeptide consisting of the amino acid sequence represented by SEQ ID NO: 8, in which the mutation may be a mutation in which an aspartic acid residue at the 1,790 residue from the N-terminal side in the polypeptide consisting of the amino acid sequence represented by SEQ ID NO: 8 is changed to a residue of an amino acid other than aspartic acid.

### TECHNICAL FIELD

This disclosure relates to a mutant strain of Trichoderma reesei, the mutant strain being capable of keeping a viscosity of a culture solution low and having an enhanced protein-producing ability, and to a method for protein production using the mutant strain.

### BACKGROUND

Trichoderma reesei is known to have a high protein-producing ability, and studies have heretofore been made on protein production using Trichoderma reesei. Trichoderma reesei specifically produces a cellulase, which is classified as a saccharifying enzyme, among proteins using cellulose, lactose, cellobiose or the like as an inducer. To enhance cellulase production amount, various investigations have hitherto been made such as genetic modifications including overexpression or deletion of a factor which controls cellulase production and optimization of cultivation conditions.

Meanwhile, fungi of the genus Trichoderma belong to the aerobic filamentous fungi, which essentially require oxygen for growth and protein production. Fungi of the genus Trichoderma are characterized in that when the fungi are cultivated in a liquid culture medium, the viscosity of the culture solution increases as the fungi grow. The increase in culture solution viscosity results in an uneven distribution of oxygen and nutrients. It is hence necessary in cultivating a fungus of the genus Trichoderma to stir the culture solution or increase the oxygen feed rate to thereby prevent the degree of saturation of oxygen dissolved in the culture solution from decreasing and keep the degree of saturation at or above a certain level. Meanwhile, use of a cultivation tank having a larger size results in a decrease in oxygen-transfer coefficient and it is hence necessary in keeping the degree of saturation of oxygen dissolved in the culture solution at or above a certain level, to further increase the number of stirring or oxygen feed rate. However, increasing the number of stirring poses a problem in that the fungus bodies suffer considerable shear damage, while increasing the oxygen feed rate poses a problem in that a larger amount of energy is necessary.

JP-T-2013-533751, JP-T-2014-513529, JP-T-2014-513530, JP-T-2014-513531, JP-T-2014-513532 and JP-T-2014-513533 disclose that when the Sfb3, Mpg1, Gas1, Seb1, Crz1, and Tps1 proteins of a fungus of the genus Trichoderma are destroyed or are reduced in protein production, the mutant strains can be cultivated by aerobic fermentation in submerged culture while maintaining a dissolved-oxygen concentration with a small number of stirring, as compared with the parent strain before mutation. WO 2017/170917 indicates that by destroying a BXL1 gene of a fungus of the gnus Trichoderma, the culture solution can be inhibited from decreasing in the degree of saturation of dissolved oxygen.

As described above, it is very important in producing a protein using Trichoderma reesei to inhibit the dissolved-oxygen concentration in the culture solution from decreasing to keep the concentration at or above a certain level.

It could therefore be helpful to acquire a mutant strain of Trichoderma reesei that renders the viscosity of the culture solution low and provide a method for protein production using the mutant strain of Trichoderma reesei.

### SUMMARY

We discovered that in producing a protein by liquid-medium cultivation of Trichoderma reesei, if the viscosity of the culture solution can be kept low, not only the energy required for stirring can be reduced but also the degree of saturation of oxygen dissolved in the culture solution can be inhibited from decreasing, even in enlarging cultivation scale.

We also discovered a gene of Trichoderma reesei that enables the culture solution to retain a low viscosity. We thus discovered that when a mutant strain of Trichoderma reesei having a mutation in a polypeptide consisting of the amino acid sequence represented by SEQ ID NO: 8 and preferably when a mutant strain further having a mutation in one or more polypeptides selected from among polypeptides consisting of the amino acid sequences represented by any of SEQ ID NOs: 6, 7, 9, and 10 is cultivated, then the culture solution can retain a low viscosity and can be inhibited from decreasing in the degree of saturation of dissolved oxygen.

We therefore provide (1) to (14):

(1) A mutant strain of Trichoderma reesei, the mutant strain having a mutation that eliminates or reduces a function of a polypeptide consisting of the amino acid sequence represented by SEQ ID NO: 8.

(2) The mutant strain according to (1), in which the mutation is a mutation in which an aspartic acid residue at the 1,791st residue from the N-terminal side in the polypeptide consisting of the amino acid sequence represented by SEQ ID NO: 8 is changed to a residue of an amino acid other than aspartic acid.

(3) The mutant strain according to (1) or (2), further having a mutation that eliminates or reduces a function of a polypeptide consisting of the amino acid sequence represented by SEQ ID NO: 6.

(4) The mutant strain according to (3), in which the mutation is a stop codon mutation that causes translation to end at the 137th position from the N-terminal side in the amino acid sequence represented by SEQ ID NO: 6.

(5) The mutant strain according to any one of (1) to (4), further having a mutation that eliminates or reduces a function of a polypeptide consisting of the amino acid sequence represented by SEQ ID NO: 7.

(6) The mutant strain according to (5), in which the mutation is a mutation that deletes a Leucine-rich repeats, ribonuclease inhibitor-like subfamily domain of the polypeptide consisting of the amino acid sequence represented by SEQ ID NO: 7.

(7) The mutant strain according to (5) or (6), in which the mutation is a frameshift mutation in an aspartic acid residue at the 297th residue from the N-terminal side in the amino acid sequence represented by SEQ ID NO: 7.

(8) The mutant strain according to any one of (1) to (7), further having a mutation in an amino acid sequence located between a GAL4-like Zn2Cys6 binuclear cluster DNA-binding domain and a fungal transcription factor regulatory middle homology region domain in a polypeptide consisting of the amino acid sequence represented by SEQ ID NO: 9.

(9) The mutant strain according to (8), in which the mutation is a mutation in which a serine residue at the 184th residue from the N-terminal side in the polypeptide consisting of the amino acid sequence represented by SEQ ID NO: 9 is changed to a residue of an amino acid other than serine.

(10) The mutant strain according to any one of (1) to (9), further having a mutation that eliminates or reduces a function of a polypeptide consisting of the amino acid sequence represented by SEQ ID NO: 10.

(11) The mutant strain according to (10), in which the mutation is a mutation that deletes a Fatty acid hydroxylase superfamily domain of the polypeptide consisting of the amino acid sequence represented by SEQ ID NO: 10.

(12) The mutant strain according to (10) or (11), in which the mutation is a frameshift mutation in an isoleucine residue at the 257th residue from the N-terminal side in the amino acid sequence represented by SEQ ID NO: 10.

(13) A method of producing a protein, the method including a step of cultivating the mutant strain according to any one of (1) to (12).

(14) A method of producing a cellulase, the method including a step of cultivating the mutant strain according to any one of (1) to (12).

Our mutant strain of Trichoderma reesei not only enables the culture solution to retain a lower viscosity but also can inhibit the culture solution from decreasing in the degree of saturation of dissolved oxygen compared to the Trichoderma reesei parent strain not having the mutation. Furthermore, this mutant strain has an unexpected effect of improving production amount of a protein, in particular a cellulase.

### BRIEF DESCRIPTION OF THE DRAWINGS

FIG. 1 shows changes with the lapse of time of the viscosity (relative value) in a culture solution of a Trichoderma reesei QM9414-H strain.

FIG. 2 shows changes with the lapse of time of the degree of saturation of dissolved oxygen in the culture solution of the Trichoderma reesei QM9414-H strain.

FIG. 3 shows changes with the lapse of time of the viscosity (relative value) in a culture solution of a Trichoderma reesei QM9414-I strain.

FIG. 4 shows changes with the lapse of time of the degree of saturation of dissolved oxygen in the culture solution of the Trichoderma reesei QM9414-I strain.

FIG. 5 shows changes with the lapse of time of the viscosity (relative value) in a culture solution of a Trichoderma reesei QM9414-J strain.

FIG. 6 shows changes with the lapse of time of the degree of saturation of dissolved oxygen in the culture solution of the Trichoderma reesei QM9414-J strain.

### DETAILED DESCRIPTION

We introduce a mutation into a parent strain of Trichoderma reesei, which is a micro-organism originally having an excellent protein-producing ability, to thereby enable the mutant strain to be cultivated in a culture solution retaining a low viscosity. The parent strain of Trichoderma reesei to be used is not limited to wild strains, and mutant strains of Trichoderma reesei which have been improved to have an increased protein-producing ability can also be favorably used as the parent strain. For example, a mutant strain having an improved protein production property obtained by performing a mutation treatment with a mutagen, UV irradiation or the like can be utilized as the parent strain of a mutant strain of Trichoderma reesei. Specific examples of mutant strains usable as the parent strain include Trichoderma parareesei (ATCC MYA-4777), which is an ancestor to Trichoderma reesei, known mutant strains derived from Trichoderma reesei such as QM6a strain (NBRC31326), QM9123 strain (ATCC24449), QM9414 strain (NBRC31329), PC-3-7 strain (ATCC66589), QM9123 strain (NBRC31327), RutC-30 strain (ATCC56765), CL-847 strain (Enzyme. Microbiol. Technol., 10, 341-346 (1988)), MCG77 strain (Biotechnol. Bioeng. Symp., 8, 89 (1978)), and MCG80 strain (Biotechnol. Bioeng., 12, 451-459 (1982)), and strains derived from these. QM6a strain, QM9419 strain, and QM9123 strain are available from NBRC (NITE Biological Resource Center), and PC-3-7 strain and RutC-30 strain are available from ATCC (American Type Culture Collection).

The mutant strain is a mutant strain of Trichoderma reesei having a mutation that eliminates or reduces a function of a polypeptide consisting of the amino acid sequence represented by SEQ ID NO: 8, and preferably is a mutant strain further having mutation(s) shown below in one or more polypeptides selected from among polypeptides consisting of amino acid sequences represented by any of SEQ ID NOs: 6, 7, 9, and 10. These mutant strains are often referred to as mutant strains of this disclosure. Strains into which the mutations have not been introduced are herein often referred to as parent strains. The mutant strain is lower in the viscosity of the culture solution and inhibits the culture solution from decreasing in the degree of saturation of dissolved oxygen, as compared with the parent strain. Thus, the energy necessary for aeration and stirring and the number of stirring can be reduced. Furthermore, since the speed of rotation for stirring can be set low, the shearing damage to the fungus bodies can be reduced. This mutant strain is more effective particularly in large-scale cultivation because reductions in the blower and the capacity of stirring motor necessary for aeration and reduction in stirring energy are attained.

Each mutation of polypeptides possessed by the mutant strain are explained in detail below.

The polypeptide consisting of the amino acid sequence represented by SEQ ID NO: 8 is a polypeptide having an overall length of 4,373 amino acid residues which is possessed by Trichoderma reesei, and in National Center for Biotechnology Information, this polypeptide has been registered as a Dynein heavy chain (EGR51787) that Trichoderma reesei QM6a strain has. Dynein is one of the motor proteins found in eucaryotes, and is a protein which moves along the surfaces of microtubes constituting the cytoskeletons such as microtubes, by energy obtained by the hydrolysis of ATP. The Dynein heavy chain is a heavy chain which constitutes Dynein and forms a main skeleton of dynein, and is a protein having the function of converting the energy obtained by the hydrolysis of ATP into a movement (D Eshel, Cytoplasmic dynein is required for normal unclear segregation in yeast, Proceedings of the National Academy of Sciences of the United States of America, Volume 90, 1993, Issue 23, P 11172-11176). Specific examples of genes encoding the polypeptide consisting of the amino acid sequence represented by SEQ ID NO: 8 include the base sequence represented by SEQ ID NO: 3 possessed by Trichoderma reesei QM6a strain.

Examples of methods of eliminating or reducing the function of the polypeptide consisting of the amino acid sequence represented by SEQ ID NO: 8 include a method of introducing a mutation that causes a total deletion of a Dynein heavy chain or a partial deletion of a Dynein heavy chain. Specific examples thereof include a method in which a frameshift mutation or a stop codon mutation is introduced into a gene sequence encoding the polypeptide consisting of the amino acid sequence represented by SEQ ID NO: 8, by a deletion, insertion, substitution and the like of a base.

The phrase “deletion of a Dynein heavy chain” means a total or partial loss of the polypeptide, a change of the whole or some of the polypeptide into different amino acid(s), or a combination of these. More specifically, that phrase means that the amino acid sequence represented by SEQ ID NO: 8 comes to have a sequence identity to the amino acid sequence of the Dynein heavy chain of 80% or less. The sequence identity thereto is preferably 50% or less, more preferably 20% or less, more preferably 10% or less, more preferably 5% or less, more preferably 3% or less, more preferably 1% or less, and most preferably 0%.

Having a mutation in the amino acid sequence constituting the Dynein heavy chain may be any of a deletion, substitution, and addition of an amino acid. Preferred is a mutation which changes the aspartic acid residue at the 1,791st residue from the N-terminal side in the amino acid sequence represented by SEQ ID NO: 8 into a residue of an amino acid other than aspartic acid. Although the amino acid residue formed through the mutation is not particularly limited, it is more preferable that the aspartic acid residue has been changed to asparagine. Specific examples of base sequences encoding the amino acid sequence in which the aspartic acid residue at the 1,791st residue from the N-terminal side in the amino acid sequence represented by SEQ ID NO: 8 has been changed to a residue of an amino acid other than aspartic acid include the base sequence represented by SEQ ID NO: 3 in which the guanine at the 5,541st base has been changed to adenine. This mutation changes the 1,791st amino acid residue from the N-terminal side in the amino acid sequence represented by SEQ ID NO: 8 from aspartic acid to asparagine.

The function of the polypeptide consisting of the amino acid sequence represented by SEQ ID NO: 8 may be reduced also by introducing a mutation which diminishes or inhibits the expression of the polypeptide. Specifically, the mutation which diminishes or inhibits the expression of the polypeptide may be one introduced into the promoter or terminator region of a gene encoding the amino acid sequence represented by SEQ ID NO: 8. In general, the promoter and terminator regions correspond to a region of hundreds of bases in length before and after the gene participating in transcription.

Whether the polypeptide consisting of the amino acid sequence represented by SEQ ID NO: 8 in a mutant strain has such a mutation that the function of this polypeptide has been reduced or eliminated can be ascertained by ascertaining that a culture solution of this mutant strain has a lower viscosity than a culture solution of the parent strain.

The polypeptide consisting of the amino acid sequence represented by SEQ ID NO: 6 is a polypeptide having an overall length of 861 amino acid residues which is possessed by Trichoderma reesei, and in National Center for Biotechnology Information, this polypeptide has been registered as an N-terminal binuclear Zn cluster-containing/DNA binding domain-containing protein (EGR44896) that Trichoderma reesei QM6a strain has. The N-terminal binuclear Zn cluster-containing/DNA binding domain-containing protein has a motif composed of two helixes consisting of a Zn2Cys6 motif binding to DNA within GAL4, which is a transcription factor, and is hence presumed to be a protein binding to DNA and having the function of a transcription factor. Specific examples of genes encoding the polypeptide consisting of the amino acid sequence represented by SEQ ID NO: 6 include the base sequence represented by Trichoderma reesei SEQ ID NO: 1.

Examples of methods of reducing or eliminating the function of the polypeptide consisting of the amino acid sequence represented by SEQ ID NO: 6 include a method of introducing a mutation that causes a total deletion of an N-terminal binuclear Zn cluster-containing/DNA binding domain-containing protein or a partial deletion of an N-terminal binuclear Zn cluster-containing/DNA binding domain-containing protein. Specific examples thereof include a method in which a frameshift mutation or a stop codon mutation is introduced into a gene sequence encoding the polypeptide consisting of the amino acid sequence represented by SEQ ID NO: 6, by a deletion, insertion, substitution and the like of a base.

The phrase “deletion of an N-terminal binuclear Zn cluster-containing/DNA binding domain-containing protein” means a total or partial loss of the polypeptide, a change of the whole or some of the polypeptide into different amino acid(s), or a combination of these. More specifically, that phrase means that the amino acid sequence represented by SEQ ID NO: 2 comes to have a sequence identity to the amino acid sequence of the N-terminal binuclear Zn cluster-containing/DNA binding domain-containing protein of 80% or less. The sequence identity thereto is preferably 50% or less, more preferably 20% or less, more preferably 10% or less, more preferably 5% or less, more preferably 3% or less, more preferably 1% or less, and most preferably 0%.

CDD Search Results of National Center for Biotechnology Information disclose that the 272nd to 307th amino acid residues from the N-terminal side are a GAL4-like Zn2Cys6 binuclear cluster DNA-binding domain and the 388th to 805th amino acid residues from the N-terminal side are a fungal transcription factor regulatory middle homology region. By causing a mutation such as deletion, substitution, or addition, to occur in an amino acid sequence located in either of these domains, the function of the polypeptide consisting of the amino acid sequence represented by SEQ ID NO: 6 can be eliminated or reduced. Specific examples thereof include a mutation in the base sequence represented by SEQ ID NO: 1 which changes the guanine at the 411st residue into adenine to thereby insert a stop codon. This mutation causes the translation to end at the 137th position from the N-terminal side in the amino acid sequence represented by SEQ ID NO: 6, thereby deleting the amino acid sequence constituting the GAL4-like Zn2Cys6 binuclear cluster DNA-binding domain and fungal transcription factor regulatory middle homology region, which perform the function of the N-terminal binuclear Zn cluster-containing/DNA binding domain-containing protein. Hence, the original function of the polypeptide as a protein is eliminated.

The function of the polypeptide consisting of the amino acid sequence represented by SEQ ID NO: 6 may be reduced also by introducing a mutation which diminishes or inhibits the expression of the polypeptide. Specifically, the mutation which diminishes or inhibits the expression of the polypeptide may be one introduced into the promoter or terminator region of a gene encoding the amino acid sequence represented by SEQ ID NO: 6. In general, the promoter and terminator regions correspond to a region of hundreds of bases in length before and after the gene participating in transcription.

Whether the polypeptide consisting of the amino acid sequence represented by SEQ ID NO: 6 in a mutant strain has such a mutation that the function of this polypeptide has been reduced or eliminated can be ascertained by ascertaining that a culture solution of this mutant strain has a lower viscosity than a culture solution of the parent strain.

The polypeptide consisting of the amino acid sequence represented by SEQ ID NO: 7 is a polypeptide having an overall length of 1,138 amino acid residues which is possessed by Trichoderma reesei, and in National Center for Biotechnology Information, this polypeptide has been registered as a predicted protein (EGR45926) that Trichoderma reesei QM6a strain has. The polypeptide consisting of the amino acid sequence represented by SEQ ID NO: 7 is a polypeptide whose function is unknown, but Conserved Domain Architecture Retrieval Tool of National Center for Biotechnology Information discloses that the 468th to 721st amino acid residues from the N-terminal side are a Leucine-rich repeats (LRRS), ribonuclease inhibitor (RI)-like subfamily domain. It is presumed from this disclosure that the polypeptide consisting of the amino acid sequence represented by SEQ ID NO: 7 forms complexes with ribonucleases to participate in stabilization of RNA or the like. Specific examples of genes encoding the polypeptide consisting of the amino acid sequence represented by SEQ ID NO: 7 include the base sequence represented by SEQ ID NO: 2 possessed by Trichoderma reesei QM6a strain.

Examples of methods of reducing or eliminating the function of the polypeptide consisting of the amino acid sequence represented by SEQ ID NO: 7 include a method of introducing a mutation that causes a total deletion of a Leucine-rich repeats, ribonuclease inhibitor-like subfamily domain or a partial deletion of a Leucine-rich repeats, ribonuclease inhibitor-like subfamily domain. Specific examples thereof include a method in which a frameshift mutation or a stop codon mutation is introduced into a gene sequence encoding the polypeptide consisting of the amino acid sequence represented by SEQ ID NO: 7, by a deletion, insertion, substitution and the like of a base.

The phrase “deletion of a Leucine-rich repeats, ribonuclease inhibitor-like subfamily domain” means a total or partial loss of the domain, a change of the whole or some of the domain into different amino acid(s), or a combination of these. More specifically, that phrase means that the amino acid sequence represented by SEQ ID NO: 7 comes to have a sequence identity to the amino acid sequence of the Leucine-rich repeats, ribonuclease inhibitor-like subfamily domain of 80% or less. The sequence identity thereto is preferably 50% or less, more preferably 20% or less, more preferably 10% or less, more preferably 5% or less, more preferably 3% or less, more preferably 1% or less, and most preferably 0%.

Specific examples of when the function of the polypeptide consisting of the amino acid sequence represented by SEQ ID NO: 7 is eliminated or reduced by a mutation such as deletion, substitution, or addition, that has occurred in the amino acid sequence represented by SEQ ID NO: 7 include a frameshift mutation due to insertion of one base residue of adenine at the 988th position in the base sequence represented by SEQ ID NO: 2. This mutation changes the 297th amino acid from the N-terminal side in the amino acid sequence represented by SEQ ID NO: 7 from aspartic acid to arginine. As a result of the succeeding frameshifts, the amino acid sequence constituting the Leucine-rich repeats, ribonuclease inhibitor-like subfamily domain disappears. Hence, the original function of the polypeptide as a protein is eliminated or reduced.

The function of the polypeptide consisting of the amino acid sequence represented by SEQ ID NO: 7 may be reduced or eliminated also by introducing a mutation which diminishes or inhibits the expression of the polypeptide. Specifically, the mutation which diminishes or inhibits the expression of the polypeptide may be one introduced into the promoter or terminator region of a gene encoding the amino acid sequence represented by SEQ ID NO: 7. In general, the promoter and terminator regions correspond to a region of hundreds of bases in length before and after the gene participating in transcription.

Whether the polypeptide consisting of the amino acid sequence represented by SEQ ID NO: 7 in a mutant strain has such a mutation that the function of this polypeptide has been reduced or eliminated can be ascertained by ascertaining that a culture solution of this mutant strain has a lower viscosity than a culture solution of the parent strain.

The polypeptide consisting of the amino acid sequence represented by SEQ ID NO: 9 is a polypeptide having an overall length of 937 amino acid residues which is possessed by Trichoderma reesei, and in National Center for Biotechnology Information, this polypeptide has been registered as a hypothetical protein (EGR48369) that Trichoderma reesei QM6a strain has. Conserved Domain Architecture Retrieval Tool of National Center for Biotechnology Information discloses that the polypeptide consisting of the amino acid sequence represented by SEQ ID NO: 9 has the following domains: the 76th to 108th amino acid residues from the N-terminal side are a domain composed of two helixes consisting of a Zn2Cys6 motif binding to a DNA possessed by GAL4, which is a transcription factor; and the 303rd to 681st amino acid residues from the N-terminal side are a fungal transcription factor regulatory middle homology region domain. We believe that the polypeptide consisting of the amino acid sequence represented by SEQ ID NO: 9 at least participates in transcription regulation in filamentous fungi. Specific examples of genes encoding the polypeptide consisting of the amino acid sequence represented by SEQ ID NO: 9 include a base sequence represented by SEQ ID NO: 4 possessed by Trichoderma reesei QM6a strain.

Examples of methods of reducing or eliminating the function of the polypeptide consisting of the amino acid sequence represented by SEQ ID NO: 9 include a method of introducing a mutation that causes: a total deletion of a GAL4-like Zn2Cys6 binuclear cluster DNA-binding domain and/or a fungal transcription factor regulatory middle homology region domain; a partial deletion of a GAL4-like Zn2Cys6 binuclear cluster DNA-binding domain and/or a fungal transcription factor regulatory middle homology region domain; a change in the configuration relationship between a GAL4-like Zn2Cys6 binuclear cluster DNA-binding domain and a fungal transcription factor regulatory middle homology region domain; or a total deletion of the polypeptide consisting of the amino acid sequence represented by SEQ ID NO: 9.

The phrase “deletion of a GAL4-like Zn2Cys6 binuclear cluster DNA-binding domain and/or a fungal transcription factor regulatory middle homology region domain” means a total or partial loss of the domain(s), a change of the whole or some of the domain(s) into different amino acid(s), or a combination of these. More specifically, that phrase means that the amino acid sequence represented by SEQ ID NO: 9 comes to have a sequence identity to the amino acid sequence of the GAL4-like Zn2Cys6 binuclear cluster DNA-binding domain or fungal transcription factor regulatory middle homology region domain of 80% or less. The sequence identity thereto is preferably 50% or less, more preferably 20% or less, more preferably 10% or less, more preferably 5% or less, more preferably 3% or less, more preferably 1% or less, and most preferably 0%.

The change in the configuration relationship between a GAL4-like Zn2Cys6 binuclear cluster DNA-binding domain and a fungal transcription factor regulatory middle homology region domain occurs due to a mutation which causes a deletion, substitution, or addition of an amino acid in an amino acid sequence located between the GAL4-like Zn2Cys6 binuclear cluster DNA-binding domain and the fungal transcription factor regulatory middle homology region domain. The GAL4-like Zn2Cys6 binuclear cluster DNA-binding domain and the fungal transcription factor regulatory middle homology region domain are called protein domains. A protein domain constitutes a part of the sequence structure of the protein and has a function. When there are a plurality of domains, a steric structure configured of the plurality of domains constitutes a part of the steric structure of the protein. Hence, a change in the configuration of the domains results in a change in the steric structure of the protein and a decrease in the function of the protein.

As stated above, it is known that even when the amino acid sequence itself of each domain does not have a mutation such as a deletion, substitution, or addition of an amino acid, the function of the protein is reduced by a mutation such as a deletion, substitution, or addition of an amino acid, that occurs in an amino acid sequence located between two domains. The amino acids located between the GAL4-like Zn2Cys6 binuclear cluster DNA-binding domain and the fungal transcription factor regulatory middle homology region domain correspond to the amino acid sequence region from the 109th to 302nd residues in the amino acid sequence shown by SEQ ID NO: 9.

The mutation in the amino acid sequence located between the GAL4-like Zn2Cys6 binuclear cluster DNA-binding domain and the fungal transcription factor regulatory middle homology region domain may be any of a deletion, substitution, and addition of an amino acid. Preferred is a mutation in which the serine residue at the 184th residue from the N-terminal side in the amino acid sequence represented by SEQ ID NO: 9 has been changed to a residue of an amino acid other than serine. Although the amino acid residue formed through the mutation is not particularly limited, it is preferable that the serine residue has been changed to asparagine. Specific examples of base sequences encoding the amino acid sequence represented by SEQ ID NO: 9 in which the serine residue at the 184th residue from the N-terminal side has been changed to a residue of an amino acid other than serine include the base sequence represented by SEQ ID NO: 4 in which the adenine at the 550th base has been changed to cytosine. This mutation changes the 184th amino acid residue from the N-terminal side in the amino acid sequence represented by SEQ ID NO: 9 from serine to arginine.

The function of the polypeptide consisting of the amino acid sequence represented by SEQ ID NO: 9 may be reduced also by introducing a mutation that diminishes or inhibits the expression of the polypeptide. Specifically, the mutation that diminishes or inhibits the expression of the polypeptide may be one introduced into the promoter or terminator region of a gene encoding the amino acid sequence represented by SEQ ID NO: 9. In general, the promoter and terminator regions correspond to a region of hundreds of bases in length before and after the gene participating in transcription.

Whether the polypeptide consisting of the amino acid sequence represented by SEQ ID NO: 9 in a mutant strain has such a mutation that the function of this polypeptide has been eliminated or reduced can be ascertained by ascertaining that a culture solution of this mutant strain has a lower viscosity than a culture solution of the parent strain.

The polypeptide consisting of the amino acid sequence represented by SEQ ID NO: 10 is a polypeptide having an overall length of 342 amino acid residues which is possessed by Trichoderma reesei, and in National Center for Biotechnology Information, this polypeptide has been registered as a predicted protein (EGR53142) that Trichoderma reesei QM6a strain has. Conserved Domain Architecture Retrieval Tool of National Center for Biotechnology Information discloses that the 147th to 264th amino acid residues from the N-terminal side in the polypeptide consisting of the amino acid sequence represented by SEQ ID NO: 10 have a Fatty acid hydroxylase superfamily domain. We believe that the polypeptide consisting of the amino acid sequence represented by SEQ ID NO: 10 has the functions such as β-carotene hydroxylase, C-5 sterol desaturase, C-4 sterol methyl oxidase, and the like, which participate in zeaxanthin synthesis and the like. Specific examples of genes encoding the polypeptide consisting of the amino acid sequence represented by SEQ ID NO: 10 include the base sequence represented by SEQ ID NO: 5 possessed by Trichoderma reesei QM6a strain.

Examples of methods of reducing or eliminating the function of the polypeptide consisting of the amino acid sequence represented by SEQ ID NO: 10 include a method of introducing a mutation that causes a total deletion of a Fatty acid hydroxylase superfamily domain, a partial deletion of a Fatty acid hydroxylase superfamily domain, or a total deletion of the polypeptide consisting of the amino acid sequence represented by SEQ ID NO: 10. Specific examples thereof include a method in which a frameshift mutation or a stop codon mutation is introduced into a gene sequence encoding the polypeptide consisting of the amino acid sequence represented by SEQ ID NO: 10, by a deletion, insertion, substitution and the like of a base.

The phrase “deletion of a Fatty acid hydroxylase superfamily domain” means a total or partial loss of the domain, a change of the whole or some of the domain into different amino acid(s), or a combination of these. More specifically, that phrase means that the amino acid sequence represented by SEQ ID NO: 10 comes to have a sequence identity to the amino acid sequence of the F-box domain of 80% or less. The sequence identity thereto is preferably 50% or less, more preferably 20% or less, more preferably 10% or less, more preferably 5% or less, more preferably 3% or less, more preferably 1% or less, and most preferably 0%.

Specific examples of when the function of the polypeptide consisting of the amino acid sequence represented by SEQ ID NO: 10 is eliminated by a mutation such as deletion, substitution, or addition, that has occurred in the amino acid sequence located in the Fatty acid hydroxylase superfamily domain include a frameshift mutation due to insertion of one base residue of guanine at the 769th position in the base sequence represented by SEQ ID NO: 5. This mutation changes the 257th amino acid from the N-terminal side in the amino acid sequence represented by SEQ ID NO: 10 from isoleucine to aspartic acid. We believe that as a result of the succeeding frameshifts, the amino acid sequence constituting the Fatty acid hydroxylase superfamily domain shortens and the original function of the polypeptide is eliminated.

The function of the polypeptide consisting of the amino acid sequence represented by SEQ ID NO: 10 may be reduced also by introducing a mutation which diminishes or inhibits the expression of the polypeptide. Specifically, the mutation which diminishes or inhibits the expression of the polypeptide may be one introduced into the promoter or terminator region of a gene encoding the amino acid sequence represented by SEQ ID NO: 10. In general, the promoter and terminator regions correspond to a region of hundreds of bases in length before and after the gene participating in transcription. Or the function can be eliminated.

Whether the polypeptide consisting of the amino acid sequence represented by SEQ ID NO: 10 in a mutant strain has such a mutation that the function of this polypeptide has been reduced or eliminated can be ascertained by ascertaining that a culture solution of this mutant strain has a lower viscosity than a culture solution of the parent strain.

To introduce such mutations into the genes, use can be made of existing genetic mutation methods such as a mutation treatment with a known mutagen or UV irradiation or the like, gene recombination such as homologous recombination using a selection marker, and a mutation by a transposon.

As stated above, the mutant strain is a mutant strain of Trichoderma reesei that has a mutation that eliminates or reduces the function of the polypeptide consisting of the amino acid sequence represented by SEQ ID NO: 8, and is preferably the mutant strain which further has the mutation in one or more polypeptides selected from among the polypeptides consisting of the amino acid sequences represented by any of SEQ ID NOs: 6, 7, 9, and 10. Examples of combinations of these mutations include the following.

A mutant strain of Trichoderma reesei in which the polypeptides consisting of the amino acid sequences represented by SEQ ID NOs: 8 and 6 have the mutation.

A mutant strain of Trichoderma reesei in which the polypeptides consisting of the amino acid sequences represented by SEQ ID NOs: 8 and 7 have the mutation.

A mutant strain of Trichoderma reesei in which the polypeptides consisting of the amino acid sequences represented by SEQ ID NOs: 8 and 9 have the mutation.

A mutant strain of Trichoderma reesei in which the polypeptides consisting of the amino acid sequences represented by SEQ ID NOs: 8 and 10 have the mutation.

A mutant strain of Trichoderma reesei in which the polypeptides consisting of the amino acid sequences represented by SEQ ID NOs: 8, 6, and 7 have the mutation.

A mutant strain of Trichoderma reesei in which the polypeptides consisting of the amino acid sequences represented by SEQ ID NOs: 8, 6, and 9 have the mutation.

A mutant strain of Trichoderma reesei in which the polypeptides consisting of the amino acid sequences represented by SEQ ID NOs: 8, 6, and 10 have the mutation.

A mutant strain of Trichoderma reesei in which the polypeptides consisting of the amino acid sequences represented by SEQ ID NOs: 8, 7, and 9 have the mutation.

A mutant strain of Trichoderma reesei in which the polypeptides consisting of the amino acid sequences represented by SEQ ID NOs: 8, 7, and 10 have the mutation.

A mutant strain of Trichoderma reesei in which the polypeptides consisting of the amino acid sequences represented by SEQ ID NOs: 8, 9, and 10 have the mutation.

A mutant strain of Trichoderma reesei in which the polypeptides consisting of the amino acid sequences represented by SEQ ID NOs: 8, 6, 7, and 9 have the mutation.

A mutant strain of Trichoderma reesei in which the polypeptides consisting of the amino acid sequences represented by SEQ ID NOs: 8, 6, 7, and 10 have the mutation.

A mutant strain of Trichoderma reesei in which the polypeptides consisting of the amino acid sequences represented by SEQ ID NOs: 8, 6, 9, and 10 have the mutation.

A mutant strain of Trichoderma reesei in which the polypeptides consisting of the amino acid sequences represented by SEQ ID NOs: 8, 7, 9, and 10 have the mutation.

A mutant strain of Trichoderma reesei in which all the polypeptides consisting of the amino acid sequences represented by SEQ ID NOs: 6 to 10 have the mutation.

A mutant strain having any of those mutation combinations can be acquired by existing genetic mutation methods such as a mutation treatment with a mutagen known to a person skilled in the art or with UV irradiation or the like, gene recombination such as homologous recombination using a selection marker, or a mutation by a transposon. Preferably, however, the mutant strain can be acquired by subjecting spores of Trichoderma reesei as a parent strain to a genetic mutation treatment with nitrosoguanidine (NTG), ethylmethanesulfonic acid (EMS), UV or the like and analyzing the genes of the resultant mutant strains to collect a mutant strain having the mutations by screening.

The mutant strain is lower in the viscosity of the culture solution and inhibits the degree of saturation of oxygen dissolved in the culture solution from decreasing, as compared with the parent strain into which the mutation has not been introduced. Thus, the energy necessary for aeration and stirring and the number of stirring can be reduced. Furthermore, since the speed of rotation for stirring can be set low, the shearing damage to the fungus bodies can be reduced. This mutant strain is more effective specifically in large-scale cultivation because reductions in the blower and the capacity of stirring motor necessary for aeration and reduction in stirring energy are attained. In addition, since the mutant strain has an enhanced protein-producing ability as compared with the parent strain into which the mutation has not been introduced, a culture solution of the mutant strain has a higher protein concentration than a culture solution obtained by cultivating the parent strain not having the mutation under the same cultivation conditions. When the protein is an enzyme, the enzyme has enhanced specific activity. The increase in protein concentration and the increase in enzyme specific activity are not particularly limited so long as the concentration and the specific activity have increased. It is, however, preferable that the increases are 20% or larger.

The viscosity of a culture solution is a value measured under the following conditions, and culture solutions are compared in viscosity by comparing maximum ones of the values measured under the following conditions. First, spores of the mutant strain of Trichoderma reesei and the parent strain, which are to be evaluated, are inoculated into preculture media (a specific example of culture compositions is as shown in Table 1 given in the Examples) to result in a concentration of 1.0×10⁵ spores per mL of the preculture medium, and cultivation is conducted on a shaker under the conditions of 28° C. and 120 rpm until the amount of fungus bodies becomes around 11 g/L. Next, each of the preculture media is inoculated, in an amount of 10% (v/v), into a main-culture medium shown in Table 2, to which Arbocel B800 (manufactured by J. Rettenmaier & Sohne) has been added in an amount of 100 g/L (w/v), and submerged culture is conducted using a 5-L jar fermenter. For measuring the viscosity of the culture medium, a digital rotational viscometer is used. The digital rotational viscometer is subjected to zero point calibration beforehand. At 17, 24, 41, 48, 65, 72, 89, and 111 hours after initiation of the cultivation or at 24, 48, 71, 89, 113, and 137 hours after initiation of the cultivation, the culture solution is sampled and a 16 mL portion of each sample is immediately introduced into a given vessel. A spindle is immersed in the culture solution and rotated at a rotational speed of 0.3 rpm to measure the resultant torque at room temperature, which is the viscosity resistance imposed on the spindle, thereby measuring the viscosity of the culture solution. The unit of the viscosity is centipoise (cP). One poise is defined as the viscosity of a fluid which, when having therein a velocity gradient of 1 cm/sec per cm, generates a stress of 1 dyne per cm² along the direction of the flow in a plane perpendicular to the direction of the velocity gradient. As the digital rotational viscometer, DV2T (BROOKFIELD Inc.) can be used. As the spindle, UL ADAPTOR (BROOKFIELD Inc.) can be used.

Compared to cultivating a parent strain into which the mutation has not been introduced under the same cultivation conditions, our mutant strain of Trichoderma reesei is lower in the viscosity of the culture solution, and the maximum viscosity during the cultivation thereof is preferably 80% or less, more preferably 70% or less, still more preferably 60% or less, most preferably 50% or less. The absolute value of the maximum viscosity during the cultivation of our mutant strain is lower than that for the parent strain by preferably 100 cP or more, more preferably 200 cP or more, more preferably 400 cP or more, more preferably 500 cP or more, still more preferably 600 cP or more, still more preferably 700 cP or more, still more preferably 800 cP or more, still more preferably 900 cP or more, especially preferably 1,000 cP or more.

The degree of saturation of oxygen dissolved in the culture solution can be calculated by measuring a rate of oxygen utilization in the culture solution. The term “rate of oxygen utilization (mM/L/hr)” means oxygen consumption rate per L of the culture solution per unit time period measured at 24 hours after cultivation initiation. A specific method for the calculation is as follows. Cultivation is conducted under constant cultivation conditions and the feeding of oxygen is stopped at 24 hours after initiation of the cultivation. Values of dissolved-oxygen (mg/L) (DO values) determined at intervals of 10 seconds are plotted, and in the resultant curve, three or more plotted points which decline logarithmically are examined for slope (A) (unit; DO/sec). The following Expression (1) is used for calculating the rate of oxygen utilization:

Rate of oxygen utilization (mM/L/hr)=(−A)×(1/32)×60×60 (1).

To measure the DO values, a commercial DO meter can be used. The DO meter to be used is not particularly limited, and any DO meter capable of accurately measuring the DO values may be used. Examples thereof include sealed DO electrodes (manufactured by ABLE Corp.) and a dissolved-oxygen sensor (manufactured by Mettler-Toledo International Inc.). The DO meter is subjected beforehand to zero point calibration and span calibration. The zero point calibration is performed using a 2% solution of sodium sulfite. The span calibration is performed by conducting aeration and stirring under the same conditions as in actual cultivation except for the existence of fungal bodies, until the culture solution becomes saturated with dissolved oxygen, thereafter ascertaining that the meter stably indicates a value, and conducting calibration to the saturated dissolved oxygen at the temperature. When the cultivation tank is pressurized in measuring DO values, it is necessary to perform a pressure correction. Furthermore, when the cultivation tank is large, it is necessary to perform a hydrostatic-pressure correction. In performing the correction, the following Expression (2) is used for calculation:

D=DO(1+α−β) (2)

D: corrected saturated dissolved oxygen
DO: saturated dissolved oxygen in pure water at 1 atm
α: gage pressure (kg/cm²)
β: hydrostatic pressure [(depth (m) of liquid at the position of DO meter)/10].

The degree of saturation of dissolved oxygen is determined by assuming that dissolved-oxygen-saturated state in the fungus-free culture medium obtained by blowing air thereinto under the same pH and temperature conditions as in the cultivation is 100%, and then calculating the proportion of the dissolved oxygen during the cultivation to a saturated dissolved oxygen. The dissolved-oxygen (mg/L) is the concentration of oxygen dissolved in the water. The phrase “saturated dissolved oxygen” means the dissolved oxygen in a culture medium which, in the state of containing no fungus bodies, has been made to have a constant dissolved oxygen by performing aeration and stirring under the same cultivation conditions as in actual cultivation. In calculating the degree of saturation of dissolved oxygen, the cultivation conditions such as aeration conditions are kept unchanged throughout the cultivation period. A decrease in oxygen demand results in an increase in the degree of saturation of dissolved oxygen. The degree of saturation of dissolved oxygen is calculated in accordance with the following Expression (3):

Degree of saturation of dissolved oxygen (%)=(dissolved oxygen during cultivation)/(saturated dissolved oxygen before cultivation initiation)×100 (3).

In comparing degrees of saturation of dissolved oxygen, minimum values are compared to each other.

When rates of oxygen utilization or degrees of saturation of dissolved oxygen are compared, use is made of results obtained through examinations conducted under the same cultivation conditions including culture medium, oxygen feed rate, stirring speed, temperature, cultivation volume, and inoculation amount. The inoculation amount in the examinations is preferably about 10% (v/v) with respect to the main-culture solution.

When the mutant strain and the parent strain are cultivated under the same dissolved-oxygen conditions, the mutant strain gives a higher minimum value of the degree of saturation of dissolved oxygen than the parent strain. The minimum value thereof is higher by preferably 5% or more, more preferably 6% or more, more preferably 7% or more, more preferably 8% or more, more preferably 9% or more, more preferably 10% or more, more preferably 11% or more, more preferably 12% or more, more preferably 13% or more, more preferably 14% or more, especially preferably 15% or more.

It is preferable that the mutant strain does not have a lower growing ability than the parent strain into which the mutation has not been introduced. A difference in growing ability can be determined by measuring the amounts of fungus bodies. The amount of fungus bodies is measured as the weight of dry fungus bodies. A 10 mL portion of the culture solution is subjected to suction filtration using a qualitative filter paper (Grade 4; GE Healthcare Co.), and the residue is dried at 100° C. together with the filter paper. The weight thereof is measured and a difference in filter-paper weight between before and after the filtration is taken as the weight of the dry fungus bodies.

Besides having the mutations described above, the mutant strain may have a mutation that improves protein production amount and/or lowers the viscosity of the culture solution to inhibit the culture solution from decreasing in the degree of saturation of dissolved oxygen. Specific examples thereof include a mutation introduced into the polypeptide consisting of the amino acid sequence represented by any of SEQ ID NOs: 11, 13, 15, 17, 19, 22, and 24.

The polypeptide consisting of the amino acid sequence represented by SEQ ID NO: 11 is a polypeptide possessed by Trichoderma reesei and has been registered at National Center for Biotechnology Information as predicted protein EGR50654 possessed by Trichoderma reesei QM6a strain. The polypeptide consisting of the amino acid sequence represented by SEQ ID NO: 11 is a polypeptide whose function is unknown, but Conserved Domain Architecture Retrieval Tool of National Center for Biotechnology Information discloses that the 95th to 277th amino acid residues from the N-terminal side have middle domain of eukaryotic initiation factor 4G domain (hereinafter referred to as MIF4G domain) and the 380th to 485th amino acid residues from the N-terminal side have MA-3 domain. The two domains, MIF4G and MA-3, are known to have the function of binding to DNAs or RNAs (Biochem., 44, 12265-12272 (2005); Mol. Cell. Biol., 1, 147-156 (2007)). We believe from those disclosures that the polypeptide consisting of the amino acid sequence represented by SEQ ID NO: 11 at least has the function of binding to a DNA and/or an RNA.

Specific examples of genes encoding the polypeptide consisting of the amino acid sequence represented by SEQ ID NO: 11 include the base sequence represented by SEQ ID NO: 12. Examples of genetic mutations which reduce or eliminate the function of EGR50654 include a total deletion of the MIF4G domain and/or MA-3 domain possessed by EGR50654, a partial deletion of the MIF4G domain and/or MA-3 domain, and a genetic mutation which changes the configuration relationship between the MIF4G domain and the MA-3 domain. Furthermore, the function of the polypeptide consisting of the amino acid sequence represented by SEQ ID NO: 11 can be reduced or eliminated also by introducing a mutation which diminishes or inhibits the expression of the polypeptide. Specific examples of the deletion of the function of the polypeptide consisting of the amino acid sequence represented by SEQ ID NO: 11 include a mutation in the base sequence represented by SEQ ID NO: 12 which deletes any of the 1,039th to 1,044th bases.

Due to the reduction or elimination of the function of the polypeptide consisting of the amino acid sequence represented by SEQ ID NO: 11, this mutant strain has an enhanced protein-producing ability as compared with the Trichoderma reesei in which the function of the polypeptide consisting of the amino acid sequence represented by SEQ ID NO: 11 is not reduced or eliminated.

The polypeptide consisting of the amino acid sequence represented by SEQ ID NO: 13 is a polypeptide possessed by Trichoderma reesei and has been registered at National Center for Biotechnology Information as predicted protein EGR44419 possessed by Trichoderma reesei QM6a strain. The polypeptide consisting of the amino acid sequence represented by SEQ ID NO: 13 is a polypeptide whose function is unknown, but Conserved Domain Architecture Retrieval Tool of National Center for Biotechnology Information discloses that the 26th to 499th amino acid residues from the N-terminal side have a Sugar (and other) Transporter domain. We believe from this disclosure that the polypeptide consisting of the amino acid sequence represented by SEQ ID NO: 13 at least participates in transport of sugar between the inside and the outside of the fungus bodies.

Specific examples of genes encoding the polypeptide consisting of the amino acid sequence represented by SEQ ID NO: 13 include the base sequence represented by SEQ ID NO: 14. Examples of genetic mutations which reduce or eliminate the function of EGR44419 include a total deletion of the Sugar (and other) Transporter domain possessed by EGR44419, a partial deletion of the Sugar (and other) Transporter domain, and a genetic mutation which changes the configuration relationship of the Sugar (and other) Transporter domain. Furthermore, the function of the polypeptide consisting of the amino acid sequence represented by SEQ ID NO: 13 can be reduced or eliminated also by introducing a mutation which diminishes or inhibits the expression of the polypeptide. Specific examples of the deletion of the function of the polypeptide consisting of the amino acid sequence represented by SEQ ID NO: 13 include a mutation in the base sequence represented by SEQ ID NO: 14 which inserts 11 base residues at the 1,415th position.

Due to the reduction or elimination of the function of the polypeptide consisting of the amino acid sequence represented by SEQ ID NO: 13, this mutant strain has an enhanced protein-producing ability and attains improved β-glucosidase specific activity, compared to the Trichoderma reesei in which the function of the polypeptide consisting of the amino acid sequence represented by SEQ ID NO: 13 is not reduce or eliminated.

The polypeptide consisting of the amino acid sequence represented by SEQ ID NO: 15 is a polypeptide possessed by Trichoderma reesei and has been registered at National Center for Biotechnology Information as EGR48910 of a beta-adaptin large subunit possessed by Trichoderma reesei QM6a strain. The polypeptide consisting of the amino acid sequence represented by SEQ ID NO: 15 is one of the proteins that constitute adaptor proteins that bind to clathrin which is widely conserved in eucaryotes, and constitute vesicles that take part in transport inside and outside the cells and inside and outside the fungus bodies (Proc. Nati. Acad. Sci. USA., 101, 14108-14113 (2004)).

Specific examples of genes encoding the polypeptide consisting of the amino acid sequence represented by SEQ ID NO: 15 include the base sequence represented by SEQ ID NO: 16. Examples of genetic mutations for EGR48910 include a mutation in the base sequence represented by SEQ ID NO: 16 that changes the cytosine at the 1,080th base into adenine.

Due to the mutation which the mutant strain has in the polypeptide consisting of the amino acid sequence represented by SEQ ID NO: 15, this mutant strain is lower in the viscosity of the culture solution thereof during liquid cultivation than the Trichoderma reesei having no mutation in the polypeptide consisting of the amino acid sequence represented by SEQ ID NO:

The polypeptide consisting of the amino acid sequence represented by SEQ ID NO: 17 is a polypeptide possessed by Trichoderma reesei, and in National Center for Biotechnology Information, this polypeptide has been registered as EGR45828 of a predicted protein possessed by Trichoderma reesei QM6a strain. The polypeptide consisting of the amino acid sequence represented by SEQ ID NO: 17 is a polypeptide whose function is unknown, but Conserved Domain Architecture Retrieval Tool of National Center for Biotechnology Information discloses that the 86th to 186th amino acid residues from the N-terminal side are a heat shock factor (HSF)-type DNA-binding domain. The HSF-type DNA-binding domain is known to have the function of binding to an upstream region of a gene encoding an HSF, which is a transcription factor controlling the expression of heat shock proteins (Cell, 65(3), 363-366 (1991)).

Specific examples of genes encoding the polypeptide consisting of the amino acid sequence represented by SEQ ID NO: 17 include the base sequence represented by SEQ ID NO: 18. Examples of genetic mutations which reduce or eliminate the function of EGR45828 include a total deletion of the HSF-type DNA-binding domain possessed by EGR45828, a partial deletion of the HSF-type DNA-binding domain, and a genetic mutation that changes the configuration relationship of the HSF-type DNA-binding domain. Furthermore, the function of the polypeptide consisting of the amino acid sequence represented by SEQ ID NO: 17 can be reduced also by introducing a mutation that diminishes or inhibits the expression of the polypeptide. Specific examples of the elimination of the function of the polypeptide consisting of the amino acid sequence represented by SEQ ID NO: 17 include a mutation in the base sequence represented by SEQ ID NO: 18 which inserts one base residue of guanine at the 85th position to cause frameshifts.

Due to the reduction or elimination of the function of the polypeptide consisting of the amino acid sequence represented by SEQ ID NO: 17, this mutant strain has an enhanced protein-producing ability as compared with the Trichoderma reesei in which the function of the polypeptide consisting of the amino acid sequence represented by SEQ ID NO: 17 is not reduced or eliminated.

The polypeptide consisting of the amino acid sequence represented by SEQ ID NO: 19 is a polypeptide possessed by Trichoderma reesei and has been registered at National Center for Biotechnology Information as a predicted protein (EGR47155) possessed by Trichoderma reesei QM6a strain. The polypeptide consisting of the amino acid sequence represented by SEQ ID NO: 19 is a polypeptide whose function is unknown, but CcnscrvcdConserved Domain Architecture Retrieval Tool of National Center for Biotechnology Information discloses that the 362nd to 553rd amino acid residues from the N-terminal side are a TLD domain. The function of the TLD domain is unknown. Specific examples of genes encoding the polypeptide consisting of the amino acid sequence represented by SEQ ID NO: 11 include the base sequence represented by SEQ ID NO: 20. Examples of genetic mutations which reduce or eliminate the function of EGR47155 include a total deletion of the TLD domain possessed by EGR47155, a partial deletion of the TLD domain, and a genetic mutation which changes the configuration relationship of the TLD domain. Furthermore, the function of the polypeptide consisting of the amino acid sequence represented by SEQ ID NO: 19 can be reduced or eliminated also by introducing a mutation which diminishes or inhibits the expression of the polypeptide. Specific examples of the elimination of the function of the polypeptide consisting of the amino acid sequence represented by SEQ ID NO: 19 include a frameshift mutation in the base sequence represented by SEQ ID NO: 20 which inserts 46 base residues represented by SEQ ID NO: 21 at the 6th position.

Due to the reduction or elimination of the function of the polypeptide consisting of the amino acid sequence represented by SEQ ID NO: 19, this mutant strain has an enhanced protein-producing ability as compared with the Trichoderma reesei in which the function of the polypeptide consisting of the amino acid sequence represented by SEQ ID NO: 19 is not reduced or eliminated.

The polypeptide consisting of the amino acid sequence represented by SEQ ID NO: 22 is a polypeptide possessed by Trichoderma reesei, and in National Center for Biotechnology Information, this polypeptide has been registered as a predicted protein (EGR48056) that Trichoderma reesei QM6a strain has. The polypeptide consisting of the amino acid sequence represented by SEQ ID NO: 22 is a polypeptide whose function is unknown, but Conserved Domain Architecture Retrieval Tool of National Center for Biotechnology Information discloses that the 130th to 172nd amino acid residues from the N-terminal side are an F-box domain. The F-box domain is known to be a domain present in proteins which control the cell cycle (Proc. Natl. Acad. Sci., 95, 2417-2422 (1998)). Specific examples of genes encoding the polypeptide consisting of the amino acid sequence represented by SEQ ID NO: 22 include the base sequence represented by SEQ ID NO: 23. Examples of genetic mutations that reduce or eliminate the function of EGR48056 include a total deletion of the F-box domain possessed by EGR48056, a partial deletion of the F-box domain, and a genetic mutation that changes the configuration relationship of the F-box domain. Furthermore, the function of the polypeptide consisting of the amino acid sequence represented by SEQ ID NO: 22 can be reduced or eliminated also by introducing a mutation that diminishes or inhibits the expression of the polypeptide. Specific examples of the elimination of the function of the polypeptide consisting of the amino acid sequence represented by SEQ ID NO: 22 include a frameshift mutation in the base sequence represented by SEQ ID NO: 23 that deletes the one cytosine base reside at the 499th residue. Due to the reduction or elimination of the function of the polypeptide consisting of the amino acid sequence represented by SEQ ID NO: 22, this mutant strain has an enhanced protein-producing ability compared to the Trichoderma reesei in which the function of the polypeptide consisting of the amino acid sequence represented by SEQ ID NO: 22 is not reduced or eliminated.

The polypeptide consisting of the amino acid sequence represented by SEQ ID NO: 24 is a polypeptide possessed by Trichoderma reesei, and in National Center for Biotechnology Information, this polypeptide has been registered as a glycosyltransferase family 41, partial (EGR46476) that Trichoderma reesei QM6a strain has. The glycosyltransferase family 41 is a protein constituted of a dimer complex (The EMBO Journal, 27, 2080-2788 (2008)), and has a function whereby a nascent protein just after translation, when passing through the Golgi complex, undergoes a change of N-acetylgalactosamine (GalNAc) into a serine or threonine residue as an amino acid residue (Biochemistry, Fourth edition, 11, 280-281 (1995)). Specific examples of genes encoding the polypeptide consisting of the amino acid sequence represented by SEQ ID NO: 24 include the base sequence represented by SEQ ID NO: 25. Examples of genetic mutations which reduce or eliminate the function of EGR46476 include a total deletion of the glycosyltransferase family 41, partial possessed by EGR46476, a partial deletion of the glycosyltransferase family 41, partial, and a genetic mutation which changes the configuration relationship of the glycosyltransferase family 41, partial. Furthermore, the function of the polypeptide consisting of the amino acid sequence represented by SEQ ID NO: 24 can be reduced or eliminated also by introducing a mutation which diminishes or inhibits the expression of the polypeptide. Specific examples of the elimination of the function of the polypeptide consisting of the amino acid sequence represented by SEQ ID NO: 24 include a mutation in the base sequence represented by SEQ ID NO: 25 that changes the cytosine residue at the 6,261st residue into adenine to thereby insert a stop codon. Due to the reduction or elimination of the function of the polypeptide consisting of the amino acid sequence represented by SEQ ID NO: 24, this mutant strain has an enhanced protein-producing ability compared to the Trichoderma reesei in which the function of the polypeptide consisting of the amino acid sequence represented by SEQ ID NO: 24 is not reduced or eliminated.

We further provide a method for protein production including a step of cultivating the mutant strain.

Methods of cultivating Trichoderma reesei are not particularly limited. For example, the strain can be cultivated by liquid culture in which a centrifuge tube, flask, jar fermenter, tank or the like is used or solid culture in which a plate or the like is used. It is preferred to cultivate Trichoderma reesei under aerobic conditions, and especially preferred of those cultivation methods is submerged culture performed in a jar fermenter or a tank while conducting aeration and stirring.

Our method can efficiently produce proteins excreted from the fungus bodies. The proteins to be produced are not particularly limited, but enzymes are preferred. More preferred are saccharifying enzymes such as cellulases, amylases, invertases, chitinases, and pectinases. Especially preferred are cellulases.

Cellulases that can be produced include various hydrolases, which include enzymes having a decomposition activity against xylan, cellulose, and hemicellulose. Specific examples thereof include cellobiohydrolase (EC 3.2.1.91), which produces cellobiose by hydrolyzing cellulose chains, endoglucanase (EC 3.2.1.4), which hydrolyzes cellulose chains from central portions thereof, β-glucosidase (EC 3.2.1.21), which hydrolyzes cellooligosaccharide and cellobiose, xylanase (EC 3.2.1.8), which is characterized by acting on hemicellulose and, in particular, on xylan, and β-xylosidase (EC 3.2.1.37), which hydrolyzes xylooligosaccharide.

As stated above, the cellulase protein concentration to ascertain the enhanced protein-producing ability of the mutant strain and the improvement in cellulase specific activity are ascertained by ascertaining that the specific activity of any of those hydrolytic enzymes has improved.

The concentration of cellulase proteins is determined in the following manner. A culture solution obtained by cultivating the Trichoderma reesei by the method is centrifuged at 15,000×g for 10 minutes to obtain a supernatant as a cellulase solution. 5 μL of a diluted cellulase solution is added to 250 μL of Quick Start Bradford protein assay (manufactured by Bio-Rad Laboratories, Inc.). The mixture is allowed to stand still at room temperature for 15 minutes and then examined for absorbance at 595 nm. The concentration of proteins contained in the saccharifying-enzyme solution is calculated on the basis of a calibration curve obtained using bovine serum albumin solutions as reference solutions.

The β-glucosidase specific activity is determined by the following method. First, 10 μL of the enzyme dilution is added to 90 μL of 50 mM acetate buffer containing 1 mM p-nitrophenyl-β-glucopyranoside (produced by Sigma-Aldrich Japan), and the mixture is allowed to react at 30° C. for 10 minutes. Then, 10 μL of 2 M sodium carbonate is added and mixed well to stop the reaction, and the increase in absorbance at 405 nm is measured. Finally, release of 1 μmol of p-nitrophenol per minute is defined as 1 U of activity to calculate the specific activity.

The β-xylosidase specific activity is determined by the following method. First, 10 μL of the enzyme dilution is added to 90 μL of 50 mM acetate buffer containing 1 mM p-nitrophenyl-β-xylopyranoside (produced by Sigma-Aldrich Japan), and the mixture is allowed to react at 30° C. for 30 minutes. Then, 10 μL of 2 M sodium carbonate is added and mixed well to stop the reaction, and the increase in absorbance at 405 nm is measured. Finally, release of 1 μmol of p-nitrophenol per minute is defined as 1 U of activity to calculate the specific activity.

The cellobiohydrolase specific activity is determined by the following method. First, 10 μL of the enzyme dilution is added to 90 μL of 50 mM acetate buffer containing 1 mM p-nitrophenyl-β-lactopyranoside (produced by Sigma-Aldrich Japan), and the mixture is allowed to react at 30° C. for 60 minutes. Thereafter, 10 μL of 2 M sodium carbonate is added and mixed well to stop the reaction, and the increase in absorbance at 405 nm is measured. Finally, release of 1 μmol of p-nitrophenol per minute is defined as 1 U of activity to calculate the specific activity.

Methods for the cultivation of the mutant strain of Trichoderma reesei are not particularly limited. For example, the mutant strain can be cultivated by liquid culture in which a centrifuge tube, flask, jar fermenter, tank or the like is used or solid culture in which a plate or the like is used. In a mutant strain of Trichoderma reesei, it is preferred to cultivate the mutant strain under aerobic conditions, and especially preferred among those cultivation methods is submerged culture performed in a jar fermenter or a tank while conducting aeration or stirring. The air flow rate is preferably about 0.1-2.0 vvm, more preferably 0.3-1.5 vvm, especially preferably 0.5-1.0 vvm. The cultivation temperature is preferably about 25-35° C., more preferably 25-31° C. The pH conditions during the cultivation are preferably pH 3.0-7.0, more preferably pH 4.0-6.0. The cultivation period is not particularly limited so long as the mutant strain can be cultivated under conditions capable of protein production, until the protein is accumulated in a recoverable amount. The cultivation period is usually 24-288 hours, preferably 24-240 hours, more preferably 36-240 hours, still more preferably 36-192 hours.

The culture medium composition in the cultivating step is not particularly limited as long as it is a culture medium composition where the Trichoderma reesei can produce a protein, and a known culture medium composition for Trichoderma reesei can be employed. As a nitrogen source, use can be made, for example, of polypeptone, bouillon, CSL, or soybean cake. An inducer for protein production may be added to the culture medium.

In producing cellulases, the mutant strain can be cultivated in a culture medium containing one or more kinds of inducers selected from the group consisting of lactose, cellulose, and xylan. To introduce cellulose or xylan, biomass containing cellulose or xylan may be added as an inducer. Specific examples of the biomass containing cellulose or xylan include not only plants such as seed plant, pteridophyte, bryophyte, algae, and water plant, but also waste building materials. The seed plants are classified into gymnosperms and angiosperms, and both can be used favorably. The angiosperms are further classified into monocotyledons and dicotyledons. Specific examples of the monocotyledons include bagasse, switchgrass, napier grass, erianthus, corn stover, corncob, rice straw, and wheat straw, and specific examples of the dicotyledons include beet pulp, eucalyptus, oak, and white birch.

As for the biomass containing cellulose or xylan, a pretreated biomass may be used. The pretreatment method is not particularly limited, but, for example, known methods such as acid treatment, sulfuric acid treatment, dilute sulfuric acid treatment, alkali treatment, hydrothermal treatment, subcritical treatment, fine grinding treatment, and steaming treatment can be used. Pulp may be used as the pretreated biomass containing cellulose or xylan.

Methods of recovering a protein contained in the culture solution where the mutant strain of Trichoderma reesei has been cultivated are not particularly limited, but the protein can be recovered by removing the fungus bodies of the Trichoderma reesei from the culture solution.

Examples of methods of removing the fungus bodies include centrifugation, membrane separation, and filter press.

Furthermore, when the culture solution in which the mutant strain of Trichoderma reesei has been cultivated is used as a protein solution without removing the fungus bodies therefrom, the culture solution is preferably treated so that the mutant strain of Trichoderma reesei cannot grow therein. Examples of treatment methods for preventing the fungus bodies from growing include heat treatment, chemical treatment, acid/alkali treatment, and UV treatment.

When the protein is an enzyme such as a cellulase, the culture solution from which the fungus bodies have been removed or which has been treated so that the fungus bodies cannot grow, as stated above, can be used directly as an enzyme solution.

### EXAMPLES

Our mutant strains and methods will be described specifically below by referring to Examples.

### Reference Example 1: Conditions for Protein Concentration Measurement

Protein concentration measuring reagent used: Quick Start Bradford protein assay, produced by Bio-Rad Laboratories, Inc.
Measuring conditions
Measuring temperature: room temperature
Protein concentration measuring reagent: 250 μL
Culture solution of filamentous fungus: 5 μL
Reaction time: 5 min
Absorbance: 595 nm
Reference: BSA

### Reference Example 2: Calculation of Degree of Saturation of Dissolved Oxygen

The degree of saturation of dissolved oxygen is determined by assuming that dissolved-oxygen-saturated state in the fungus-free culture medium obtained by blowing air thereinto under the same pH and temperature conditions as in the cultivation is 100%, and then calculating the proportion of the dissolved oxygen during the cultivation to a saturated dissolved oxygen. As a DO meter, sealed dissolved-oxygen electrode SDOC-12F-L120 (manufactured by ABLE Corp.) was used.

### Reference Example 3: Measurement of Viscosity of Culture Solution

For measurement of viscosity in the collected culture solution, culture solution samples collected at 39, 48, 62, 72, 86, 96, and 111 hours after initiation of cultivation were examined for viscosity (cP) using digital rotational viscometer DV2T and spindle LV-1 (manufactured by BROOKFIELD Inc.) at a rotational speed set at 0.3 rpm.

### Reference Example 4: Measurement of Amount of Fungus Bodies

The amount of fungus bodies contained in a culture solution was determined by subjecting the culture solution to suction filtration with a filter paper and taking the difference in the weight of the filter paper with dry fungus bodies between before and after the suction filtration as the amount of the fungus bodies.

### Reference Example 5: Conditions for Determination of Specific Activity of Cellulases Conditions for Determination of β-Glucosidase Specific Activity

Substrate: p-nitrophenyl-β-glucopyranoside (produced by Sigma-Aldrich Japan)
Reaction solution: 90 μL of 50 mM acetate buffer containing 1 mM p-nitrophenyl-β-glucopyranoside
Enzyme dilution: 10 μL
Reaction temperature: 30° C.
Reaction time: 10 min
Reaction terminator: 10 μL of 2 M sodium carbonate
Absorbance: 405 nm

### Conditions for Determination of β-Xylosidase Specific Activity

Substrate: p-nitrophenyl-β-xylopyranoside (produced by Sigma-Aldrich Japan)
Reaction solution: 90 μL of 50 mM acetate buffer containing 1 mM p-nitrophenyl-β-xylopyranoside
Enzyme dilution: 10 μL
Reaction temperature: 30° C.
Reaction time: 10 min
Reaction terminator: 10 μL of 2 M sodium carbonate
Absorbance: 405 nm

### Conditions for Determination of Cellobiohydrolase Specific Activity

Substrate: p-nitrophenyl-β-lactopyranoside (produced by Sigma-Aldrich Japan)
Reaction solution: 90 μL of 50 mM acetate buffer containing 1 mM p-nitrophenyl-β-lactopyranoside
Enzyme dilution 10 μL
Reaction temperature: 30° C.
Reaction time: 10 min
Reaction terminator: 10 μL of 2 M sodium carbonate
Absorbance: 405 nm

### Example 1

Preparation of Trichoderma reesei Mutant Strain in which Polypeptide Consisting of the Amino Acid Sequence Represented by SEQ ID NO: 6 has been Deleted:

### Method of Preparing Mutant Strain

A Trichoderma reesei mutant strain in which the function of the polypeptide consisting of the amino acid sequence represented by SEQ ID NO: 6 has been eliminated is prepared in the following manner. A gene represented by SEQ ID NO: 1 that encodes the polypeptide consisting of the amino acid sequence represented by SEQ ID NO: 6 is destroyed by replacing the gene with acetamide as a selection marker and with acetamidase gene (amdS) capable of decomposing acetamide as a selection marker gene. A DNA fragment consisting of the gene sequence represented by SEQ ID NO: 26 is prepared to eliminate the function of the polypeptide consisting of the amino acid sequence represented by SEQ ID NO: 6, and Trichoderma reesei QM9414 strain is transformed with the DNA fragment, thereby preparing the Trichoderma reesei mutant strain in which the function of the polypeptide consisting of the amino acid sequence represented by SEQ ID NO: 6 has been eliminated. By this method, a Trichoderma reesei mutant strain is obtained in which the base sequence represented by SEQ ID NO: 1 has been deleted. To allow a DNA fragment consisting of the base sequence represented by SEQ ID NO: 1 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 27 and 28, and the resulting amplified fragment is treated with restriction enzymes AfIII and KpnI to obtain a DNA fragment for use as the upstream DNA fragment. In addition, PCR is conducted using oligo DNAs represented by SEQ ID NOs: 29 and 30, and the resulting amplified fragment is treated with restriction enzymes MluI and SpeI to obtain a DNA fragment for use as the downstream DNA fragment. The upstream and downstream DNA fragments are introduced into an amdS-containing plasmid by using restriction enzymes AfIII and KpnI and restriction enzymes MluI and SpeI, respectively, to construct a plasmid for mutation introduction. The plasmid for mutation introduction is treated with restriction enzymes AfIII and SpeI, and the Trichoderma reesei QM9414 strain is transformed with the obtained DNA fragment which is shown by SEQ ID NO: 26. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Example 2

Preparation of Trichoderma reesei Mutant Strain in which Polypeptide Consisting of the Amino Acid Sequence Represented by SEQ ID NO: 7 has been Deleted:

### Method of Preparing Mutant Strain

A Trichoderma reesei mutant strain in which the function of the polypeptide consisting of the amino acid sequence represented by SEQ ID NO: 7 has been eliminated is prepared in the following manner. A gene represented by SEQ ID NO: 2 that encodes the polypeptide consisting of the amino acid sequence represented by SEQ ID NO: 7 is destroyed by replacing the gene with acetamide as a selection marker and with acetamidase gene (amdS) capable of decomposing acetamide as a selection marker gene. A DNA fragment consisting of the gene sequence represented by SEQ ID NO: 31 is prepared to eliminate the function of the polypeptide consisting of the amino acid sequence represented by SEQ ID NO: 7, and Trichoderma reesei QM9414 strain is transformed with the DNA fragment, thereby preparing the Trichoderma reesei mutant strain in which the function of the polypeptide consisting of the amino acid sequence represented by SEQ ID NO: 7 has been eliminated. By this method, a Trichoderma reesei mutant strain is obtained in which the base sequence represented by SEQ ID NO: 2 has been deleted. To allow a DNA fragment consisting of the base sequence represented by SEQ ID NO: 2 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 32 and 33, and the resulting amplified fragment is treated with restriction enzymes AfIII and NotI to obtain a DNA fragment for use as the upstream DNA fragment. In addition, PCR is conducted using oligo DNAs represented by SEQ ID NOs: 34 and 35, and the resulting amplified fragment is treated with restriction enzymes SwaI and AscI to obtain a DNA fragment for use as the downstream DNA fragment. The upstream and downstream DNA fragments are introduced into an amdS-containing plasmid by using restriction enzymes AfIII and NotI and restriction enzymes SwaI and AscI, respectively, to construct a plasmid for mutation introduction. The plasmid for mutation introduction is treated with restriction enzymes AfIII and AscI, and the Trichoderma reesei QM9414 strain is transformed with the obtained DNA fragment which is shown by SEQ ID NO: 31. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Example 3

Preparation of Trichoderma reesei Mutant Strain in which Polypeptide Consisting of the Amino Acid Sequence Represented by SEQ ID NO: 8 has been Deleted:

### Method of Preparing Mutant Strain

A Trichoderma reesei mutant strain in which the function of the polypeptide consisting of the amino acid sequence represented by SEQ ID NO: 8 has been eliminated is prepared in the following manner. A gene represented by SEQ ID NO: 3 that encodes the polypeptide consisting of the amino acid sequence represented by SEQ ID NO: 8 is destroyed by replacing the gene with acetamide as a selection marker and with acetamidase gene (amdS) capable of decomposing acetamide as a selection marker gene. A DNA fragment consisting of the gene sequence represented by SEQ ID NO: 36 is prepared to eliminate the function of the polypeptide consisting of the amino acid sequence represented by SEQ ID NO: 8, and Trichoderma reesei QM9414 strain is transformed with the DNA fragment, thereby preparing the Trichoderma reesei mutant strain in which the function of the polypeptide consisting of the amino acid sequence represented by SEQ ID NO: 8 has been eliminated. By this method, a Trichoderma reesei mutant strain is obtained in which the base sequence represented by SEQ ID NO: 3 has been deleted. To allow a DNA fragment consisting of the base sequence represented by SEQ ID NO: 3 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 37 and 38, and the resulting amplified fragment is treated with restriction enzymes AfIII and NotI to obtain a DNA fragment for use as the upstream DNA fragment. In addition, PCR is conducted using oligo DNAs represented by SEQ ID NOs: 39 and 40, and the resulting amplified fragment is treated with restriction enzymes MluI and SpeI to obtain a DNA fragment for use as the downstream DNA fragment. The upstream and downstream DNA fragments are introduced into an amdS-containing plasmid by using restriction enzymes AfIII and NotI and restriction enzymes MluI and SpeI, respectively, to construct a plasmid for mutation introduction. The plasmid for mutation introduction is treated with restriction enzymes AfIII and SpeI, and the Trichoderma reesei QM9414 strain is transformed with the obtained DNA fragment which is shown by SEQ ID NO: 36. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Preparation and Evaluation of Mutant Strain

By the method described above, Trichoderma reesei mutant strain QM9414-J was acquired in which the polypeptide consisting of the amino acid sequence represented by SEQ ID NO: 8 had been deleted.

### Example 4

Preparation of Trichoderma reesei Mutant Strain in which Polypeptide Consisting of the Amino Acid Sequence Represented by SEQ ID NO: 9 has been Deleted:

### Method of Preparing Mutant Strain

A Trichoderma reesei mutant strain in which the function of the polypeptide consisting of the amino acid sequence represented by SEQ ID NO: 9 has been eliminated is prepared in the following manner. A gene represented by SEQ ID NO: 4 that encodes the polypeptide consisting of the amino acid sequence represented by SEQ ID NO: 9 is destroyed by replacing the gene with acetamide as a selection marker and with acetamidase gene (amdS) capable of decomposing acetamide as a selection marker gene. A DNA fragment consisting of the gene sequence represented by SEQ ID NO: 41 is prepared to eliminate the function of the polypeptide consisting of the amino acid sequence represented by SEQ ID NO: 9, and Trichoderma reesei QM9414 strain is transformed with the DNA fragment, thereby preparing the Trichoderma reesei mutant strain in which the function of the polypeptide consisting of the amino acid sequence represented by SEQ ID NO: 9 has been eliminated. By this method, a Trichoderma reesei mutant strain is obtained in which the base sequence represented by SEQ ID NO: 4 has been deleted. To allow a DNA fragment consisting of the base sequence represented by SEQ ID NO: 4 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 42 and 43, and the resulting amplified fragment is treated with restriction enzymes AfIII and NotI to obtain a DNA fragment for use as the upstream DNA fragment. In addition, PCR is conducted using oligo DNAs represented by SEQ ID NOs: 44 and 45, and the resulting amplified fragment is treated with restriction enzymes MluI and SpeI to obtain a DNA fragment for use as the downstream DNA fragment. The upstream and downstream DNA fragments are introduced into an amdS-containing plasmid by using restriction enzymes AfIII and NotI and restriction enzymes MluI and SpeI, respectively, to construct a plasmid for mutation introduction. The plasmid for mutation introduction is treated with restriction enzymes AfIII and SpeI, and the Trichoderma reesei QM9414 strain is transformed with the obtained DNA fragment which is shown by SEQ ID NO: 41. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Example 5

Preparation of Trichoderma reesei Mutant Strain in which Polypeptide Consisting of the Amino Acid Sequence Represented by SEQ ID NO: 10 has been Deleted:

### Method of Preparing Mutant Strain

A Trichoderma reesei mutant strain in which the function of the polypeptide consisting of the amino acid sequence represented by SEQ ID NO: 10 has been eliminated is prepared in the following manner. A gene represented by SEQ ID NO: 5 that encodes the polypeptide consisting of the amino acid sequence represented by SEQ ID NO: 10 is destroyed by replacing the gene with acetamide as a selection marker and with acetamidase gene (amdS) capable of decomposing acetamide as a selection marker gene. A DNA fragment consisting of the gene sequence represented by SEQ ID NO: 46 is prepared to eliminate the function of the polypeptide consisting of the amino acid sequence represented by SEQ ID NO: 10, and Trichoderma reesei QM9414 strain is transformed with the DNA fragment, thereby preparing the Trichoderma reesei mutant strain in which the function of the polypeptide consisting of the amino acid sequence represented by SEQ ID NO: 10 has been eliminated. By this method, a Trichoderma reesei mutant strain is obtained in which the base sequence represented by SEQ ID NO: 5 has been deleted. To allow a DNA fragment consisting of the base sequence represented by SEQ ID NO: 5 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 47 and 48, and the resulting amplified fragment is treated with restriction enzymes AfIII and NotI to obtain a DNA fragment for use as the upstream DNA fragment. In addition, PCR is conducted using oligo DNAs represented by SEQ ID NOs: 49 and 50, and the resulting amplified fragment is treated with restriction enzymes SalI and SphI to obtain a DNA fragment for use as the downstream DNA fragment. The upstream and downstream DNA fragments are introduced into an amdS-containing plasmid by using restriction enzymes AfIII and NotI and restriction enzymes SalI and SphI, respectively, to construct a plasmid for mutation introduction. The plasmid for mutation introduction is treated with restriction enzymes AfIII and SphI, and the Trichoderma reesei QM9414 strain is transformed with the obtained DNA fragment which is shown by SEQ ID NO: 46. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Example 6

Cultivation Test of Trichoderma reesei Mutant Strains

### Preculture

After spores of each of the Trichoderma reesei mutant strains prepared in Examples 1 to 5 are diluted with physiological saline to be 1.0×10⁷/mL, 2.5 mL of the diluted spore solution is inoculated into 250 mL of the preculture medium shown in Table 1 that has been placed in a 1 L baffled flask, and is incubated on a shaker under the conditions of 28° C. and 120 rpm for 72 hours. Trichoderma reesei QM9414 strain is used as a control to conduct the same experiment.

TABLE 1

| | | |
|---|---|---|
| Glucose | 20 | g |
| 5 × Mandel's solution* | 200 | mL |
| 10 × Ammonium tartrate solution** | 100 | mL |
| Corn steep liquor | 50 | g |
| Trace element solution*** | 1 | mL |
| Tween 80 | 0.5 | mL |

| | |
|---|---|
| PE-M | 1 mL (per 1 L) |

*The 5 × Mandel's solution has the following composition.
7 g/L (NH₄)₂SO₄
10 g/L KH₂PO₄
2 g/L CaCl₂•2H₂O
1.5 g/L MgSO₄•7H₂O
**The 10 × Ammonium tartrate solution contains 92 g/L ammonium tartrate.
***The trace element solution has the following composition.
0.3 g/L H₃BO₃
1.3 g/L (NH₄)₆Mo₇O₂₄•4H₂O
5 g/L FeCl₃•6H₂O
2 g/L CuSO₄•5H₂O
0.4 g/L MnCl₂•4H₂O
10 g/L ZnCl₂

### Main Culture

Arbocel B800 (produced by J. Rettenmaier & Sohne) is added to the main-culture medium shown in Table 2, and an investigation of submerged culture is conducted using a 5 L jar fermenter (produced by ABLE & Biott Co., Ltd.).

The preculture solutions of the Trichoderma reesei QM9414 strain and the Trichoderma reesei mutant strains prepared in Examples 1 to 5 are each inoculated in an amount of 250 mL into 2.5 L of the main-culture medium to which Arbocel B800 has been added.

After the inoculation of each preculture medium into the main-culture medium, submerged culture is performed under the cultivation conditions of 28° C., 700 rpm, and an air flow rate of 100 mL/min while regulating the pH to 5.0.

TABLE 2

| | | |
|---|---|---|
| Arbocel B800 (produced by J. Rettenmaier & Sohne) | 100 | g |
| 5 × Mandel's solution* | 200 | mL |
| Corn steep liquor | 25 | g |
| Trace element solution*** | 1 | mL |
| Tween 80 | 0.5 | mL |

| | |
|---|---|
| PE-M | 1 mL (per 1 L) |

*Same as in Table 1.
***Same as in Table 1.

### Collection of Culture Solutions

20 mL portion of each of the culture solutions is collected at intervals from initiation of the cultivation to 120 hours thereafter, at which the cultivation is terminated. A part of the collected culture solution is centrifuged under the conditions of 15,000×g and 4° C. for 10 minutes to obtain a supernatant. The supernatant is filtered with a 0.22 μm filter, and the filtrate is used as a cellulase solution in the following experiments.

### Determination of Protein Concentration

The cellulase protein concentration of each of the culture solutions that have been collected at 120 hours after initiation of the cultivation is determined using the technique shown in Reference Example 1. As a result, the culture solutions of the Trichoderma reesei mutant strains prepared in Examples 1 to 5 have higher protein concentrations than the culture solution of the Trichoderma reesei QM9414 strain. In particular, the QM9414-J strain acquired in Example 3, in which the polypeptide consisting of the amino acid sequence represented by SEQ ID NO: 8 had been eliminated, gave a protein concentration which was 1.3 times, in terms of relative value, the protein concentration obtained with the Trichoderma reesei QM9414 strain. Determination of Degrees of Saturation of Oxygen Dissolved in Culture Solutions

Using the technique described in Reference Example 2, the degree of saturation of dissolved oxygen of each of the culture solutions of the Trichoderma reesei mutant strains prepared in Examples 1 to 5 is determined with the lapse of time. As a result, the degree of saturation of dissolved oxygen of each of the culture solutions of the Trichoderma reesei mutant strains prepared in Examples 1 to 5 is higher than that of the culture solution of the Trichoderma reesei QM9414 strain.

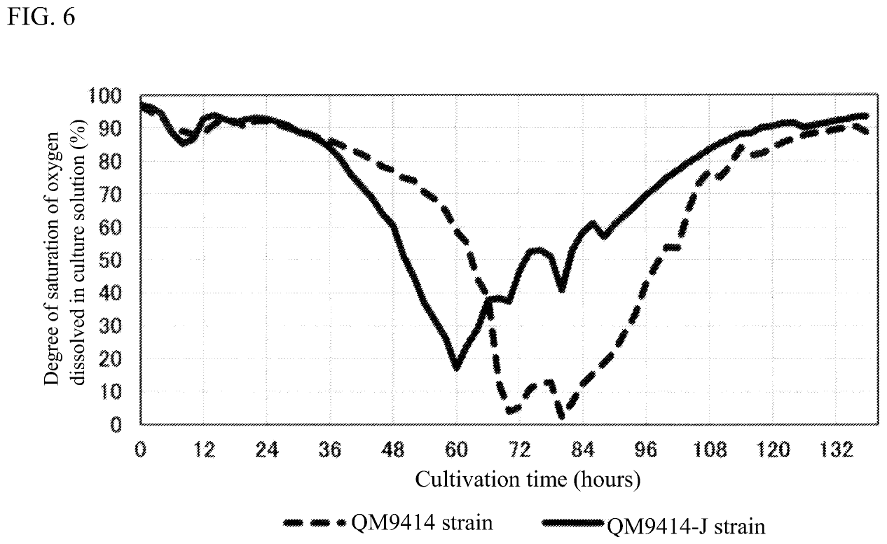

FIG. 6 shows changes with the lapse of time of the dissolved oxygen of the culture solution of QM9414 and that of the culture solution of the QM9414-J strain acquired in Example 3. The dissolved-oxygen concentrations in the culture solutions of the QM9414 strain and the QM9414-J strain had minimum values respectively at about 80 hours and at about 60 hours after initiation of the cultivation. The minimum dissolved-oxygen concentration for the QM9414-J strain was higher by about 20% than the minimum dissolved-oxygen concentration for the parent QM9414 strain.

### Measurement of Viscosity of the Culture Solutions

Using the technique described in Reference Example 3, the viscosity of each of the culture solutions of the Trichoderma reesei mutant strains prepared in Examples 1 to 5 is measured with the lapse of time. As a result, the maximum viscosities of the culture solutions of the Trichoderma reesei mutant strains prepared in Examples 1 to 5 are lower than that of the culture solution of the Trichoderma reesei QM9414 strain.

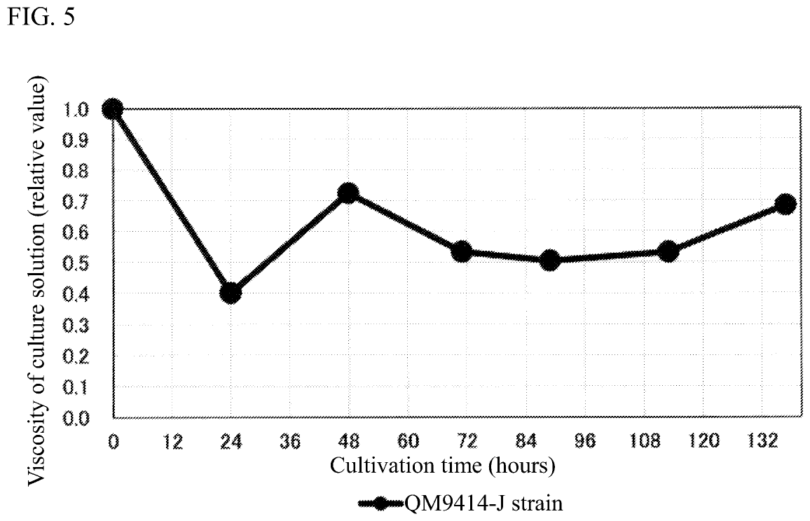

FIG. 5 shows relative values of the viscosity of the culture solution of the QM9414-J strain acquired in Example 3, with respect to the values for the Trichoderma reesei QM9414 strain which were taken as 1. As a result, the viscosity of the culture solution of the QM9414-J strain was lower than that of the culture solution of the QM9414 strain during the cultivation period. The culture solutions of the QM9414 strain and the QM9414-J strain had maximum viscosities at about 71 hours after initiation of the cultivation. The maximum viscosity for the QM9414-J strain was as low as about 40% of the maximum viscosity for the parent QM9414 strain.

### Measurement of Amount of Fungus Bodies

Using the technique described in Reference Example 4, the amount of fungus bodies is measured just after the cultivation in Example 6 (Preculture). As a result, no difference in fungus body amount is observed between each of the Trichoderma reesei mutant strains in which the function of the polypeptide consisting of the amino acid sequence represented by any of SEQ ID NOs: 6 to 10 has been eliminated and the Trichoderma reesei QM9414 strain. In particular, no difference in fungus body amount was able to be observed between the QM9414-J strain acquired in Example 3, in which the polypeptide consisting of the amino acid sequence represented by SEQ ID NO: 8 had been deleted, and the Trichoderma reesei QM9414 strain. Determination of Enzyme Activities

The culture solutions collected during the cultivation are examined for cellulase specific activities, i.e., the specific activities of β-glycosidase, β-xylosidase, and cellobiohydrolase, under the conditions shown in Reference Example 5. In determining the specific activity, an increase in absorbance at 405 nm is measured, and release of 1 μmol of the substrate per minute is defined as 1 U of activity to calculate the specific activity. As a result, the culture solutions obtained by cultivating the Trichoderma reesei mutant strains in which the function of the polypeptides consisting of the amino acid sequences represented by SEQ ID NOs: 6 to 10 has been eliminated are higher in the three specific activities than the culture solution obtained by cultivating the Trichoderma reesei QM9414 strain. In particular, the QM9414-J strain acquired in Example 3 gave relative values of β-glycosidase specific activity, β-xylosidase specific activity, and cellobiohydrolase specific activity which were respectively 1.2 times, 1.2 times, and 1.1 time the corresponding specific activities obtained with the Trichoderma reesei QM9414 strain.

### Example 7

Preparation of Trichoderma reesei Mutant Strain in which Polypeptides Consisting of the Amino Acid Sequences Represented by SEQ ID NOs: 6 to 8 have Mutation:

A QM9414-G strain, which was a strain obtained by passage culture of Trichoderma reesei QM9414 strain, was subjected to a genetic mutation treatment to acquire a QM9414-H strain as a mutant strain. The genetic mutation treatment was conducted in the following manner. Spores of the QM9414-G strain were inoculated into the preculture medium shown in Table 1 so that 1.0×10⁵ spores were inoculated per mL of the preculture medium. 15 mL of the preculture medium was incubated for a half day and then centrifuged to recover the spores. The recovered spores were suspended in a Tris-maleate buffer (pH 6.0) to give a 10 mL spore solution, and 0.5 mL of an NTG solution obtained by dissolution with a Tris-maleate buffer (pH 6.0) to result in a concentration of 1.0 g/L was added thereto. The resultant mixture was held at 28° C. for 100 minutes to perform the genetic mutation treatment. The spores that had undergone the genetic mutation treatment were recovered by centrifuging, subsequently rinsed with a Tris-maleate buffer (pH 6.0) three times, and finally suspended as genetic-mutation-treated spores in 10 mL of a Tris-maleate buffer (pH 6.0).

The genetic-mutation-treated spores were added to an agar medium prepared by adding crystalline cellulose. The size of halos, which surrounded colonies and indicated regions where the crystalline cellulose had been decomposed by cellulases, was used as an index to select a QM9414-H strain which had formed a large halo.

The QM9414-G strain and the QM9414-H strain were genetically analyzed. As a result, it was ascertained that the QM9414-G strain retained the genes encoding the polypeptides consisting of the amino acid sequences represented by SEQ ID NOs: 6 to 10, whereas the QM9414-H strain had the three mutations (1) to (3) shown below.

(1) The guanine at the 411st residue in the base sequence represented by SEQ ID NO: 1 had changed to adenine. This is a mutation that inserts a stop codon at the 137th position in the amino acid sequence represented by SEQ ID NO: 6.

(2) One base residue of adenine had been inserted at the 988th position in the base sequence represented by SEQ ID NO: 2. This is a mutation that inserts frameshifts into the 297th and succeeding positions in the amino acid sequence represented by SEQ ID NO: 7.

(3) The guanine at the 5,541st residue in the base sequence represented by SEQ ID NO: 3 had changed to adenine. This is a mutation that changes the aspartic acid at the 1,791st residue in the amino acid sequence represented by SEQ ID NO: 8 into asparagine.

### Example 8

Cultivation Test of Trichoderma reesei Mutant Strain

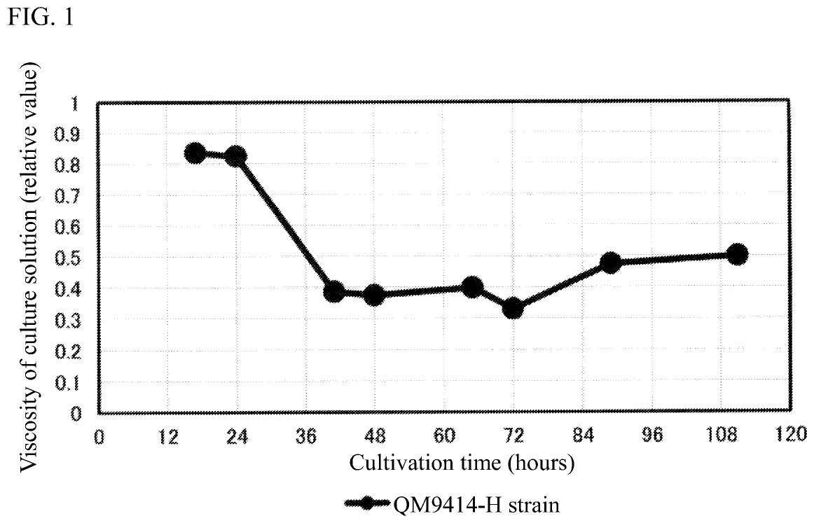

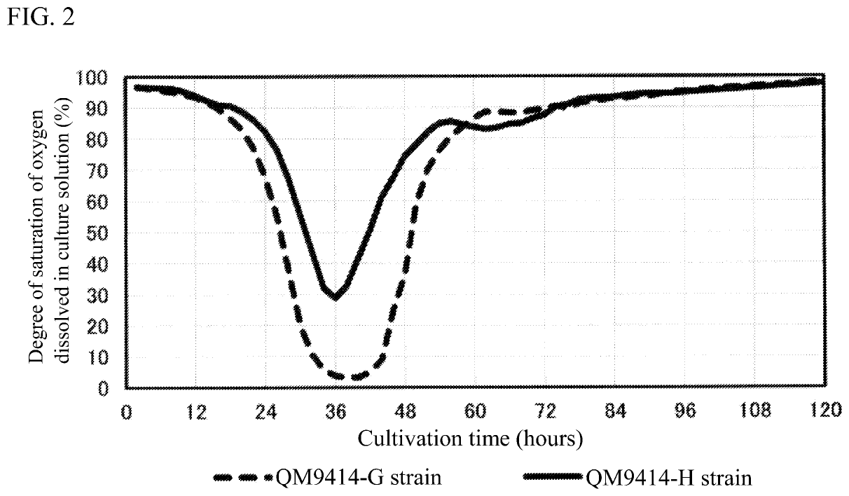

The QM9414-H strain acquired in Example 7 was cultivated in the same manner as in Example 6, and the maximum viscosity (cP) of the culture solution and the minimum degree of saturation of dissolved oxygen (%) of the culture solution were determined under the conditions shown in Reference Examples 2 and 3. The QM9414-G strain was used as a control. FIG. 1 shows relative values of the viscosity of the culture solution of the QM9414-H strain, with respect to the values for the QM9414-G strain which were taken as 1. FIG. 2 shows changes, with the lapse of time during the cultivation period, of the dissolved oxygen of the culture solutions of the QM9414-G strain and the QM9414-H strain.

As a result, the viscosity of the culture solution of the QM9414-H strain was lower than that of the culture solution of the QM9414-G strain during the cultivation period. The culture solutions of the QM9414-H strain and the QM9414-G strain had maximum viscosities respectively at about 24 hours and at about 41 hours after initiation of the cultivation. The maximum viscosity for the QM9414-H strain was as low as about 40% of the maximum viscosity for the parent QM9414-G strain.

Furthermore, the QM9414-H strain was higher in the dissolved-oxygen concentration in the culture solution than the QM9414-G strain. The dissolved-oxygen concentrations in the culture solutions of the QM9414-H strain and the QM9414-G strain had minimum values both at about 36 hours after initiation of the cultivation. At 36 hours after initiation of the cultivation, the dissolved-oxygen concentration for the QM9414-H strain was higher by about 25% than that for the parent QM9414-G strain.

### Example 9

Preparation of Trichoderma reesei Mutant Strain in which Polypeptides Consisting of the Amino Acid Sequences Represented by SEQ ID NOs: 9 and 10 have Mutation:

The QM9414-H strain, which was a strain of passage culture of Trichoderma reesei QM9414 strain and acquired in Example 7, was subjected to a genetic mutation treatment to acquire a QM9414-I strain as a mutant strain. The genetic mutation treatment was conducted in the following manner. Spores of the QM9414-H strain were inoculated into the preculture medium shown in Table 1 so that 1.0×10⁵ spores were inoculated per mL of the preculture medium. 15 mL of the preculture medium was incubated for a half day and then centrifuged to recover the spores. The recovered spores were suspended in a Tris-maleate buffer (pH 6.0) to give a 10 mL spore solution, and 0.5 mL of an NTG solution obtained by dissolution with a Tris-maleate buffer (pH 6.0) to result in a concentration of 1.0 g/L was added thereto. The resultant mixture was held at 28° C. for 100 minutes to perform the genetic mutation treatment. The spores that had undergone the genetic mutation treatment were recovered by centrifuging, subsequently rinsed with a Tris-maleate buffer (pH 6.0) three times, and finally suspended as genetic-mutation-treated spores in 10 mL of a Tris-maleate buffer (pH 6.0). The genetic-mutation-treated spores were added to an agar medium prepared by adding crystalline cellulose. The size of halos, which surrounded colonies and indicated regions where the crystalline cellulose had been decomposed by cellulases, was used as an index to select a QM9414-I strain which had formed a large halo.

The QM9414-H strain and the QM9414-I strain were genetically analyzed. As a result, it was ascertained that the QM9414-H strain retained the genes encoding the polypeptides consisting of the amino acid sequences represented by SEQ ID NOs: 9 and 10, whereas the QM9414-I strain had the two mutations shown below.

(1) The adenine at the 550th residue in the base sequence represented by SEQ ID NO: 4 had changed to cytosine. This is a mutation that changes the serine at the 184th residue in the amino acid sequence represented by SEQ ID NO: 9 into arginine.

(2) One base residue of guanine had been inserted at the 769the position in the base sequence represented by SEQ ID NO: 5. This is a mutation that inserts frameshifts into the 257th and succeeding positions in the amino acid sequence represented by SEQ ID NO: 10.

### Example 10

Cultivation Test of Trichoderma reesei Mutant Strain

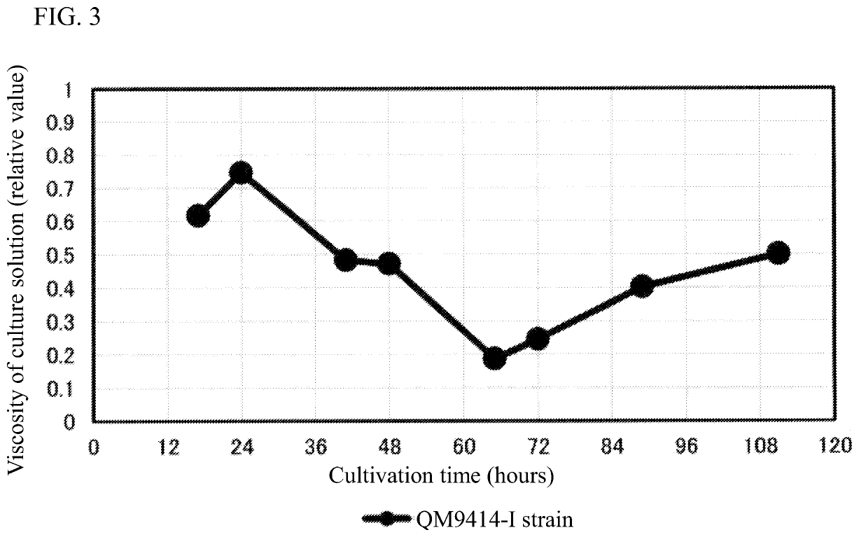

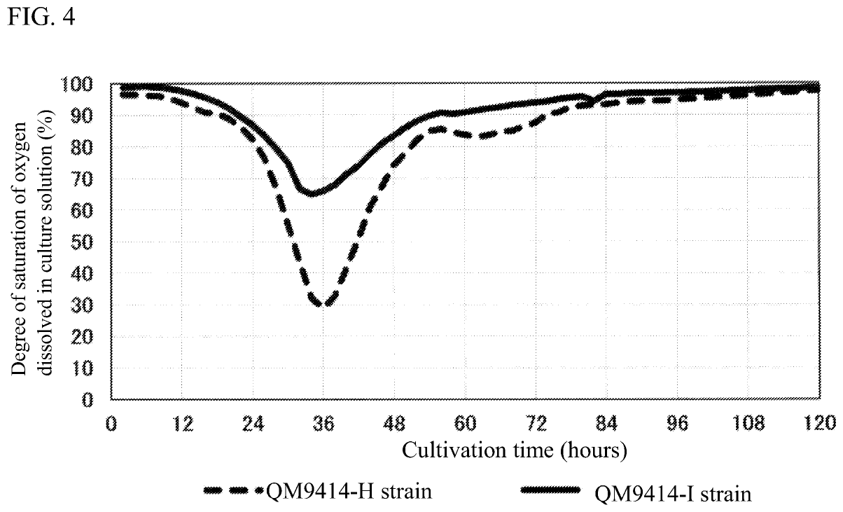

The QM9414-I strain acquired in Example 9 was cultivated in the same manner as in Example 6, and the maximum viscosity of the culture solution, the minimum degree of saturation of oxygen dissolved in the culture solution, the protein concentration in the culture solution, and the cellulase specific activities thereof were determined under the conditions shown in Reference Examples 1, 2, 3, and 5. The QM9414-H strain was used as a control. FIG. 3 shows relative values of the viscosity of the culture solution of the QM9414-I strain, with respect to the values for the QM9414-H strain which were taken as 1. FIG. 4 shows changes, with the lapse of time through the cultivation period, of the dissolved oxygen of the culture solutions of the QM9414-H strain and the QM9414-I strain.

As a result, the viscosity of the culture solution of the QM9414-I strain was lower than that of the culture solution of the QM9414-H strain. The culture solutions of the QM9414-I strain and the QM9414-H strain had maximum viscosities at about 24 hours after initiation of the cultivation. At 24 hours after the initiation, the viscosity for the QM9414-I strain was as low as about 75% of the viscosity for the parent QM9414-H strain.

Furthermore, the QM9414-I strain was higher in the dissolved-oxygen concentration in the culture solution than the QM9414-H strain. The dissolved-oxygen concentrations in the culture solutions of the QM9414-H strain and the QM9414-I strain had minimum values both at about 36 hours after the initiation. At 36 hours after the initiation, the dissolved-oxygen concentration for the QM9414-I strain was higher by about 37% than that for the parent QM9414-H strain.

In addition, the QM9414-I strain attained a protein concentration of 1.11 times, a (3-glucosidase specific activity of 1.07 times, a 3-xylosidase specific activity of 1.40 times, and a cellobiohydrolase specific activity of 1.03 times compared to those attained by the QM9414-H strain.

1-14. (canceled)

15. A mutant strain of Trichoderma reesei, the mutant strain having a mutation that eliminates or reduces a function of a polypeptide consisting of the amino acid sequence represented by SEQ ID NO: 8.

16. The mutant strain according to claim 15, wherein the mutation is a mutation in which an aspartic acid residue at the 1,791st residue from the N-terminal side in the polypeptide consisting of the amino acid sequence represented by SEQ ID NO: 8 is changed to a residue of an amino acid other than aspartic acid.

17. The mutant strain according to claim 15, further having a mutation that eliminates or reduces a function of a polypeptide consisting of the amino acid sequence represented by SEQ ID NO: 6.

18. The mutant strain according to claim 17, wherein the mutation is a stop codon mutation that causes translation to end at the 137th position from the N-terminal side in the amino acid sequence represented by SEQ ID NO: 6.

19. The mutant strain according to claim 15, further having a mutation that eliminates or reduces a function of a polypeptide consisting of the amino acid sequence represented by SEQ ID NO: 7.

20. The mutant strain according to claim 19, wherein the mutation deletes a Leucine-rich repeats, ribonuclease inhibitor-like subfamily domain of the polypeptide consisting of the amino acid sequence represented by SEQ ID NO: 7.

21. The mutant strain according to claim 19, wherein the mutation is a frameshift mutation in an aspartic acid residue at the 297th residue from the N-terminal side in the amino acid sequence represented by SEQ ID NO: 7.

22. The mutant strain according to claim 15, further having a mutation in an amino acid sequence located between a GAL4-like Zn2Cys6 binuclear cluster DNA-binding domain and a fungal transcription factor regulatory middle homology region domain in a polypeptide consisting of the amino acid sequence represented by SEQ ID NO: 9.

23. The mutant strain according to claim 22, wherein the mutation is a mutation in which a serine residue at the 184th residue from the N-terminal side in the polypeptide consisting of the amino acid sequence represented by SEQ ID NO: 9 is changed to a residue of an amino acid other than serine.

24. The mutant strain according to claim 15, further having a mutation that eliminates or reduces a function of a polypeptide consisting of the amino acid sequence represented by SEQ ID NO: 10.

25. The mutant strain according to claim 24, wherein the mutation deletes a Fatty acid hydroxylase superfamily domain of the polypeptide consisting of the amino acid sequence represented by SEQ ID NO: 10.

26. The mutant strain according to claim 24, wherein the mutation is a frameshift mutation in an isoleucine residue at the 257th residue from the N-terminal side in the amino acid sequence represented by SEQ ID NO: 10.

27. A method of producing a protein, the method comprising a step of cultivating the mutant strain according to claim 15.

28. A method of producing a cellulase, the method comprising a step of cultivating the mutant strain according to claim 15.
